(19) United States
(12) Patent Application Publication
(10) Pub. No.: US 2023/0087785 A1
(43) Pub. Date: Mar. 23, 2023

(54) Leucine-Rich Repeat Kinase 2 Allosteric Modulators

(71) Applicants: VIB VZW, Gent (BE); VRIJE UNIVERSITEIT BRUSSEL, Brussel (BE); RIJKSUNIVERSITEIT GRONINGEN, Groningen (NL); DEUTSCHES ZENTRUM FÜR NEURODEGENERATIVE ERKRANKUNGEN E.V., Bonn (DE)

(72) Inventors: Wim Versées, Merchtem (BE); Ranjan Kumar Singh, Oudergem (BE); Arjan Kortholt, Groningen (NL); Christian-Johannes Gloeckner, Rottenburg (DE)

(21) Appl. No.: 17/801,921

(22) PCT Filed: Feb. 22, 2021
(86) PCT No.: PCT/EP2021/054339

§ 371 Date: Aug. 24, 2022

Related U.S. Application Data
(60) Provisional application No. 62/981,200, filed on Feb. 25, 2020.

(30) Foreign Application Priority Data
Mar. 25, 2020 (EP) 20165463.9
Dec. 8, 2020 (EP) 20212466.5

(51) Int. Cl. C07K 16/40; G01N 33/68

(52) U.S. Cl. CPC C07K 16/40; G01N 33/6893; C07K 2317/31; C07K 2317/569; C07K 2317/92; G01N 2333/912; G01N 2800/2835

(57) ABSTRACT

The present invention relates to binding agents of human Leucine-rich Repeat Kinase 2 (LRRK2). More particular, allosteric modulators of LRRK2 activity have been identified, for targeting LRRK2 in human cells, while leaving LRRK2 subcellular localisation unaffected. Even more specifically, protein binding agents for allosteric modulation of LRRK2 kinase activity are disclosed, comprising immunoglobulin single variable domains (ISVDs) binding to human LRRK2 with nanomolar affinity. The invention thus reveals means and methods for a novel LRRK2 targeting approach through allosteric modulation of its activity for use in treatment of LRRK2-related pathologies, such as Parkinson's disease, as well as for use in detection of LRRK2 in vitro and in vivo, and for use as a diagnostic.

### CROSS REFERENCE TO RELATED APPLICATIONS

This application is a national phase entry under 35 U.S.C. § 371 of International Patent Application PCT/EP2021/054339, filed Feb. 22, 2021, designating the United States of America and published in English as International Patent Publication WO 2021/170540 on Sep. 2, 2021, which claims the benefit under Article 8 of the Patent Cooperation Treaty to U.S. Provisional Application Ser. No. 62/981,200, filed Feb. 25, 2020, and claims the benefit to European Patent Application Serial No. 20165463.9, filed Mar. 25, 2020, and claims the benefit to European Patent Application Serial No. 20212466.5, filed Dec. 8, 2020, the entireties of which are hereby incorporated by reference.

### FIELD OF THE INVENTION

The present invention relates to binding agents of human Leucine-rich Repeat Kinase 2 (LRRK2). More particular, allosteric modulators of LRRK2 activity have been identified, for targeting LRRK2 in human cells, while leaving LRRK2 subcellular localisation unaffected. Even more specifically, protein binding agents for allosteric modulation of LRRK2 kinase activity are disclosed, comprising immunoglobulin single variable domains (ISVDs) binding to human LRRK2 with nanomolar affinity. The invention thus reveals means and methods for a novel LRRK2 targeting approach through allosteric modulation of its activity for use in treatment of LRRK2-related pathologies, such as Parkinson's disease, as well as for use in detection of LRRK2 in vitro and in vivo, and for use as a diagnostic.

### BACKGROUND

Mutations in the gene encoding Leucine-Rich Repeat Kinase 2 (LRRK2) are the most common genetic cause of Parkinson's disease (PD) and are inherited in an autosomal dominant manner [62-64], while LRRK2 gene variants have also been associated with the idiopathic forms of PD [65,66]. PD is a neurodegenerative movement disorder [61] for which prevalence is expected to globally increase in the future [58-60], and besides symptomatic treatment, there is a lack of therapeutic options. Moreover, mutations in LRRK2 have been associated with other diseases including chronic inflammatory conditions, such as Crohn's disease [67-68]. LRRK2 is a large multi-domain protein (285 kDa), belonging to the ROCO protein family. The enzymatic core of the protein consists of an active GTPase domain (Roc), a dimerization module (COR) and an active Ser/Thr protein kinase domain (KD) [13, 16, 69, 70]. Several Rab GTPases have been identified as physiological substrates of the LRRK2 kinase [25-27], which furthermore provides for autophosphorylation activity of LRRK2 [28]. The monomeric form of LRRK2 protein predominantly occurs in the cytosol, with reduced kinase activity, while the dimeric LRRK2 form at the membrane shows higher kinase activity [19, 20, 71, 72]. The most common pathogenic mutations in LRRK2 are clustered within the Roc-COR and kinase domains. Importantly, several PD mutations lead to a decrease in GTPase and/or an increase in kinase activity [16, 50, 73-78]. Most notably, autophosphorylation of Serine-1292 [28] and Rab protein phosphorylation [25] are increased by pathogenic LRRK2 variants, and particularly by the most common G2019S mutation located in the kinase domain. These findings support the idea that LRRK2 mutations cause PD through a gain-of-function mechanism and suggest that modulation of LRRK2 protein function could be a promising drug target [47, 79, 80]. Since PD mutants result in an increase in LRRK2 kinase activity, drug development so far has mainly focused on developing kinase inhibitors targeting the ATP-binding pocket. Several inhibitors have been identified that are selective for LRRK2 kinase activity. However, long-term inhibition of LRRK2 with these ATP-competitive inhibitors has been reported to lead to severe kidney abnormalities in rodents and an accumulation of lamellar bodies in type II pneumocytes in the non-human primate lung [46, 47, 81-83], and the current LRRK2 kinase inhibitors will require further optimization and testing.

A high resolution structure of the catalytic half of LRRK2 has recently been disclosed [36], as a starting point for exploring alternative routes and binding modes for therapeutic targeting of LRRK2, though so far no full length human LRRK2 has been described, and only parts of the protein, such as the catalytic domain have been resolved at atomic resolution. Current LRRK2-specific ATP-competitive inhibitors are mainly suggested to be type I kinase inhibitors and are known to pronounce toxicity risks. This hurdle for clinical development of such orthosteric kinase inhibitors indicates the need for a different approach via allosteric modulation of LRRK2 kinase activity. So far, there has been identified only one small compound, the natural vitamin B12 and derivatives, that are capable of inhibiting LRRK2 activity via a non-ATP-competitive mechanism [43]. These Vitamin B12 compound directly binds LRRK2 via contact sites in the kinase domain and are believed to act as a mixed-type allosteric inhibitor capable of affecting ATP binding to LRRK2, through disruption of LRRK2 dimerization. The compound has been shown to contact the kinase domain involving the compounds' adenosyl moiety, the bulky corrin ring of cobalamin and the DMZ base, stretching a novel binding site of LRRK2 kinase domain, and may thereby alter its conformation and dimerization status. Whether these Vitamin B12 derivatives provide for a new therapeutic class with reduced toxicity and high specificity is still to be seen.

So, there is still a need to find alternative LRRK2 modulators with high specificity and alternative mode of action in order to overcome the clinical hurdles for therapeutic purposes and treatment of LRRK2-related disorders such as Parkinson's among other diseases.

### SUMMARY OF THE INVENTION

The present invention is based on a novel approach of targeting the multiple enzymatic functions and regulatory mechanisms of LRRK2 in an allosteric way, using compounds that bind outside the ATP pocket, thereby exploring the advantage of increased selectivity and lower toxicity [84, 85]. The present invention provides for immunoglobulin single variable domain (ISVD) antibodies (specifically, VHHs or Nanobodies, as used interchangeably herein) modulating the LRRK2 protein dynamics, regulation and activity. ISVDs were identified as allosteric modulators of human LRRK2 kinase activity. A wide range of VHH families binding to different LRRK2 domains with a variety of affinities have been selected in view of their advantageous properties as a potential therapeutic or diagnostic. Several of the VHHs of the present invention robustly inhibit LRRK2 kinase activity, both in cells and in vitro, while others significantly increase LRRK2 activity in cells. Moreover, LRRK2-inhibiting Nbs with complete kinase inhibiting activity, as well as with specific inhibition of phosphorylation of Rab substrates are discerned herein. Interestingly, a subset of the Nbs inhibit kinase activity while not binding directly on the kinase domain and acting as mixed (non-competitive)-type inhibitors, demonstrate the allosteric modulatory role of those Nbs in kinase inhibition. Surprisingly, and in contrast to currently available kinase inhibitors, the Nbs do not induce formation of LRRK2 filaments on microtubules in cells, moreover, some Nbs even revert this adverse side-effect, pinpointing that Nbs provide for a novel type of allosteric LRRK2 modulating and binding agents that act entirely different from previously identified inhibitors such as the currently available ATP-competitive kinase inhibitors, and thereby provide novel therapeutic opportunities in the fight against Parkinson's disease.

So in a first aspect the invention relates binding agents specifically binding human Leucine-rich Repeat Kinase 2 (LRRK2), wherein binding within a cell preserves LRRK2 unassociated with microtubules. The fact that a large panel of LRRK2-binding ISVDs allosterically act via binding to different protein domains or via binding on the interface of various domains, not involving direct binding to the kinase domain or more specifically to the catalytic site of the kinase domain, differs from the classical ATP-competitive LRRK2 inhibitors or from compounds that target the GTP binding site, surprisingly resulting in the effect that, in contract to the orthosteric ATP-competitive LRRK2 binders, no induction of LRRK2 filaments at the microtubules takes place. Furthermore, said LRRK2 binders have a binding affinity for LRRK2 in the nanomolar range, and/or corresponding to a KD value in the range of 200 nM or lower, more preferably in the nano- to picomolar range. In a specific embodiment, said LRRK2 binders of the present invention affect LRRK2 kinase activity in cells and/or in vitro.

Specific embodiments herein relate to LRRK2 allosteric modulators, wherein said modulators structurally comprise a small compound, a chemical, a protein, peptide or peptidomimetic, or an antibody, antibody mimetic, a single domain antibody, or more specifically, an immunoglobulin single variable domain (ISVD), a Nanobody, or any active antibody fragment.

More specifically, the LRRK2 allosteric binders comprising ISVDs as described herein, which have LRRK2 inhibiting activity hold promise as a novel therapeutic strategy to treat PD and/or other LRRK2-linked diseases (including inflammatory diseases such as IBD, more specifically Crohn's disease) using a mechanism of action not described for LRRK2-related therapeutics so far, as tools or as therapeutic proteins, even as nucleic acids or delivered as a vector.

One embodiment relates to said LRRK2 allosteric modulators specifically binding LRRK2 in a non-ATP-competitive binding mode, i.e. at a binding site different from the ATP-catalytic site. Preferably, said LRRK2 allosteric modulator specifically binds LRRK2 on a binding site constituted of LRRK2 amino acids that do not exclusively or solely comprise amino acids present in the LRRK2 kinase catalytic site, or in the kinase domain. Alternatively, said LRRK2 allosteric modulator preferentially binds LRRK2 to one or more binding sites of protein domains different from the kinase domain, i.e. to one or more protein domains selected from the group of domains consisting of the N-terminal domains (armadillo, ankyrin repeats or LRR), the Roc, the COR and the WD40 domain. In another embodiment, a combination of binding to the kinase domain and any of said other domains is possible. In another embodiment, the Nb may bind the kinase domain on a binding site which is different from the ATP-binding site. In one embodiment, said LRRK2 binding agents modulating LRRK2 activity may increase LRRK2 kinase activity, as compared to a control, or alternative may decrease or inhibit or block kinase activity, as compared to a control or in the absence of said binding agent. In another embodiment, both kinase and GTP activity may be modulated by the same binding agent, in an opposite manner (i.e. inhibit kinase is increase GTPase, and vice versa).

In one embodiment, said allosteric modulator of LRRK2, comprises an ISVD specifically binding human LRRK2, comprising the structure as in the formula herein: FR1-CDR1-FR2-CDR2-FR3-CDR3-FR4, depicting 4 Framework regions and 3 complementarity determining regions. In a specific embodiment, said ISVD for allosteric modulation of LRRK2 kinase activity comprises CDR1 which consists of the sequence selected from the group of CDR1 sequences of the ISVDs disclosed herein in SEQ ID NO: 1-19 (wherein said CDR regions are annotated as in Table 3-4 or alternatively as exemplified in FIG. 10); and a CDR2 which consists of the sequence selected from the group of CDR2 sequences of the ISVDs disclosed herein in SEQ ID NO: 1-19, and a CDR3 which consists of the sequence selected from the group of CDR3 sequences of the ISVDs disclosed herein in SEQ ID NO: 1-19. Thus LRRK2-specific allosteric binders are described herein comprising an ISVD wherein the CDR1, CDR2 and CDR3 regions are selected from those CDR1, CDR2 and CDR3 regions of a sequence selected from the group of sequences of SEQ ID NO: 1 to 19, wherein the CDR regions are annotated according to Kabat, MacCallum, IMGT, AbM, or Chothia, as defined further herein and as known in the art. Alternatively, said LRRK2 binding agents comprise at least one ISVD wherein: CDR1 comprises sequence selected from the group of CDR1 sequences of SEQ ID NO: 23-41, and CDR2 comprises a sequence selected from the group of CDR2 sequences of SEQ ID NO: 42-60, and CDR3 comprises a sequence selected from the group of CDR3 sequences of SEQ ID NO: 61-79.

A further specific embodiment discloses said LRRK2-specific allosteric binding agents, comprising an ISVD which comprises any of the sequences of SEQ ID NO: 1 to 19, or variants thereof containing the identical CDR sequences and substitutions in the FR regions, but with at least 85% sequence identity of SEQ ID NO:1-19. Another embodiment relates to said LRRK2 allosteric modulators comprising an ISVD that is a humanized variant of any of the sequences selected from the group of SEQ ID NO:1-19, or a homologue with at least 85% identity thereof wherein the CDR sequences are identical to the CDRs in SEQ ID NO:1-19.

Another embodiment relates to a LRRK2 allosteric binding agent as defined herein, comprising an ISVD and inhibiting or blocking LRRK2 kinase activity in cells, and/or specifically preventing LRRK2-mediated substrate phosphorylation in cells. A specific embodiment relates to agents specifically preventing or inhibiting Rab substrate phosphorylation. An alternative embodiment relates to said LRRK2 modulator as defined herein, comprising an ISVD which increases LRRK2 kinase activity in cells, as compared to a control. A further embodiment relates to an LRRK2 allosteric modulator as described herein, comprising an ISVD, which prevents LRRK2 association to microtubules in a cell, especially when in the presence of an ATP-competitive LRRK2 kinase inhibitor compound.

Another embodiment of the present invention discloses a multi-specific or multivalent binding agent as an allosteric modulator of LRRK2 activity, which comprises at least one LRRK2 allosteric modulator as disclosed herein. Another embodiment discloses said multi-specific or multivalent binding agent as an allosteric modulator of LRRK2 activity, which comprises at least two of said LRRK2 allosteric modulators as disclosed herein. Another embodiment of the present invention discloses a multi-specific binding agent as an allosteric modulator of LRRK2 activity, which comprises said LRRK2 allosteric modulator as disclosed herein and a further binding agent with a different target specificity, or alternatively a further binding agent of LRRK2. In a specific embodiment said multi-specific or multivalent LRRK2 allosteric binding agent comprises at least one ISVD specifically binding LRRK2 as disclosed herein.

Another aspect of the invention relates to nucleic acid molecules coding for the LRRK2 allosteric modulators as described herein. Other embodiments comprise a vector containing said nucleic acid molecule as described herein, which may be a cloning or expression vector, as well as a delivery vehicle such as a viral, lentiviral or adenoviral vector.

A further aspect provides for a pharmaceutical composition comprising the LRRK2 allosteric modulator or multi-specific LRRK2 binding agent comprising a LRRK2 allosteric modulator as disclosed herein. Alternatively, a pharmaceutical composition is provided comprising said (multi-specific) LRRK2 allosteric modulator as disclosed herein, and an ATP-competitive LRRK2 kinase inhibitor compound. In a specific embodiment, said pharmaceutical composition is provided comprising said (multi-specific) LRRK2 allosteric modulator as disclosed herein, and an ATP-competitive LRRK2 kinase inhibitor compound of the type I ATP-competitive kinase inhibitors.

Another aspect of the invention relates to the LRRK2 allosteric modulator as disclosed herein, or the nucleic acid molecule or vector provided herein, or the pharmaceutical compositions described herein, for use as a medicament. Specific embodiments relate to the LRRK2 allosteric modulator as disclosed herein, or the nucleic acid molecule or vector provided herein, or the pharmaceutical compositions described herein, for use as a medicament, or more specifically for use as in treatment of a subject to treat LRRK2-related disorders. Another specific embodiment so relates to the LRRK2 allosteric modulator as disclosed herein, or the nucleic acid molecule or vector provided herein, or the pharmaceutical compositions described herein, for use in treatment of Parkinson's disease.

A further embodiment relates to said LRRK2-specific binding agents or ISVDs as disclosed herein, or the nucleic acid molecule or vector encoding said LRRK2 binding agents as provided herein, or the pharmaceutical compositions described herein, for use in a diagnostic assay or in medical in vivo imaging.

A final aspect relates to an in vitro method of detecting human LRRK2 protein in a sample, more specifically in a biological sample. Said method of detecting LRRK2 in sample may comprise the steps of reacting the sample with a LRRK2-specific binding agent or ISVD, as disclosed herein, and detecting the localization and distribution of said LRRK2-specific ISVD in said biological sample, bound to LRRK2. For said method, the LRRK2 binding agent or ISVD as disclosed herein may comprise a detectable label or a tag. Another embodiment relates to an in vitro method for detecting the presence, absence or level of LRRK2 protein in a sample, the method comprising: contacting a sample with the LRRK2-specific binding agent or ISVD, which optionally comprises a label, and detecting the presence or absence or level of said interacting or bound LRRK2-specific ISVD to its LRRK2 binding site. Optionally, said sample is a body fluid, such as cerebrospinal fluid, or is a protein extract or cell lysate.

Finally, the binding agents disclosed herein may also be used in a screening assay, as a tool, or in drug discovery.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIGS. 1A and 1B. Mapping of the domain specificity of the purified Nanobodies using ELISA. (FIG. 1A) Domain mapping of the 42 purified Nbs using an ELISA on either full-length LRRK2 or the RocCOR, Roc, COR-B or kinase-WD40 constructs. Negative controls with either no antigen coated in the ELISA plate or no Nbs added are also included. Each ELISA signal is the average of three ELISA setups. (FIG. 1B) Schematic summary of the results obtained in (FIG. 1A). Note that two Nbs did not show any binding above background, which are therefore not included in this scheme.

FIGS. 2A-2D. Influence of a selected set of 18 Nbs on the kinase activity of the LRRK2(G2019S) variant in HEK293T cells.

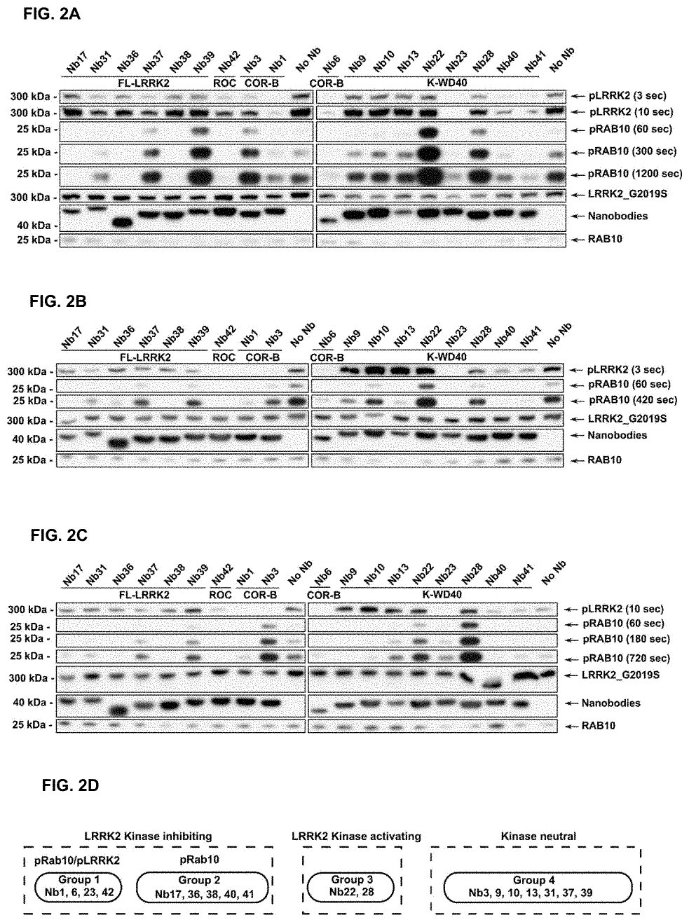

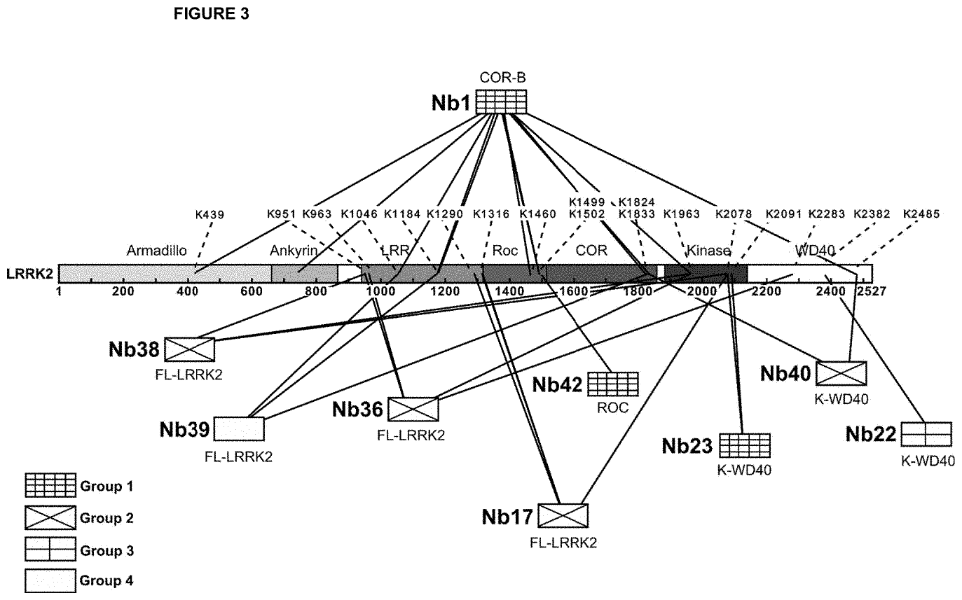

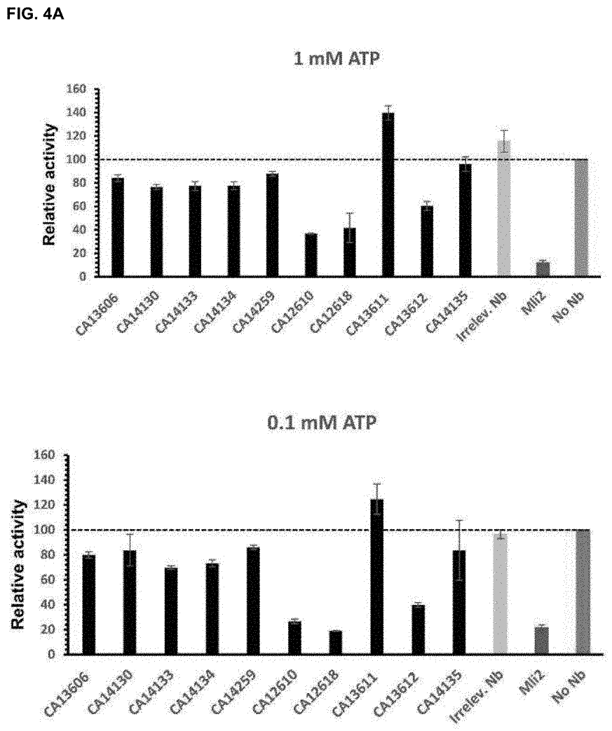

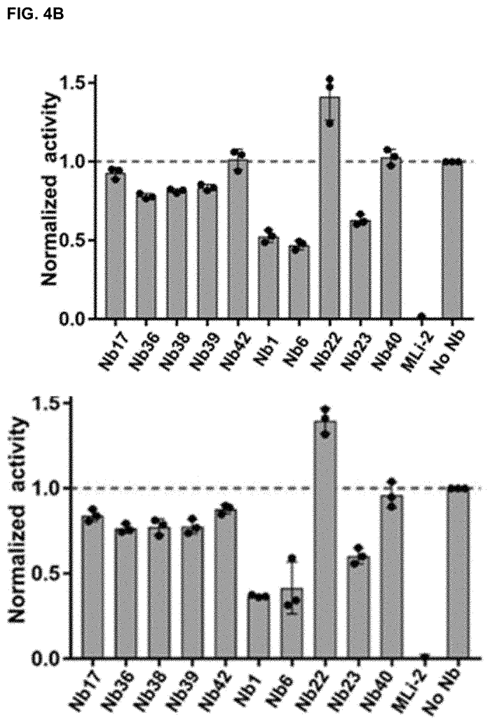

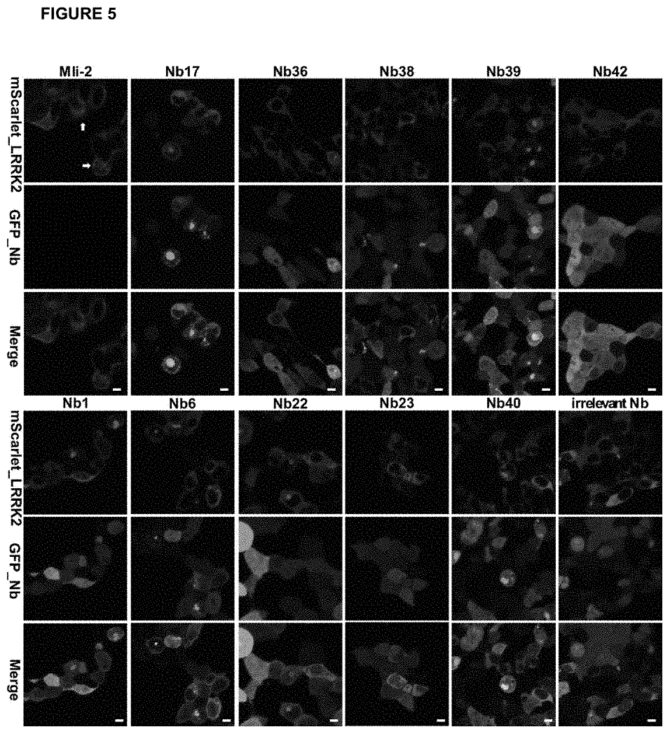

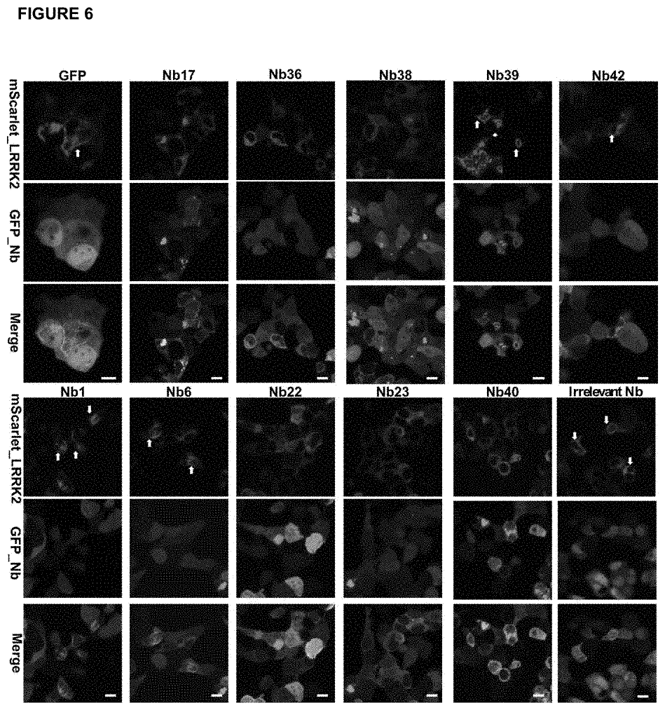

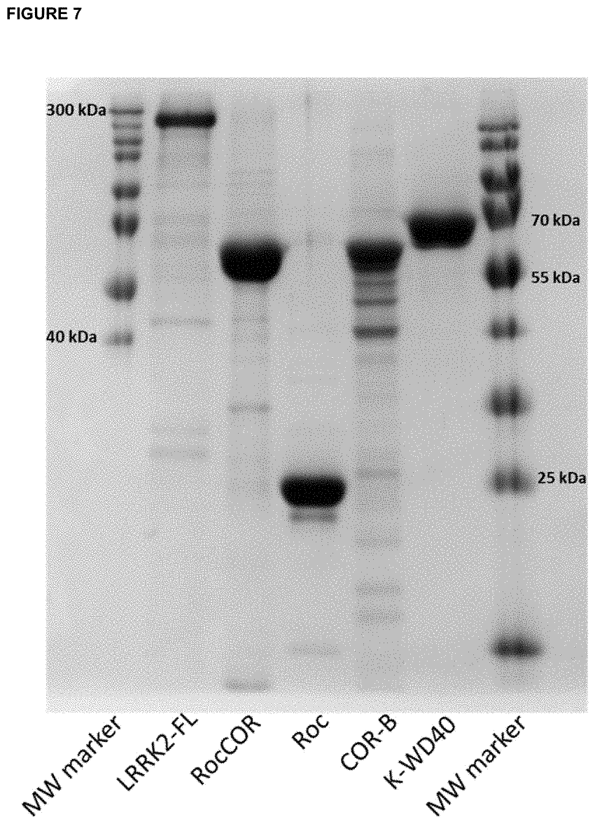

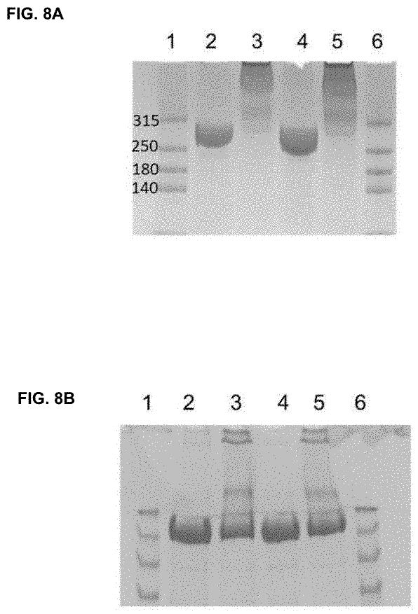

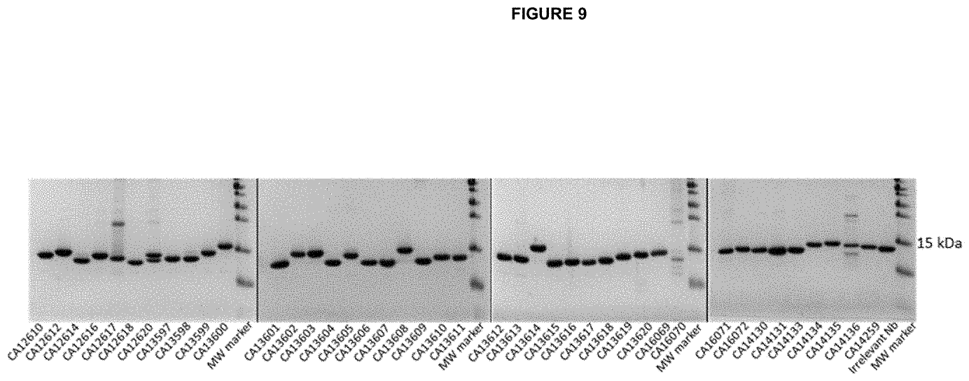

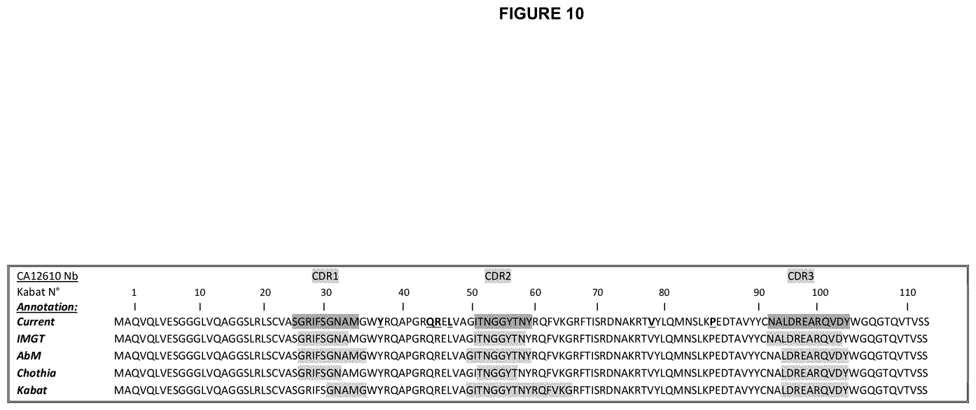

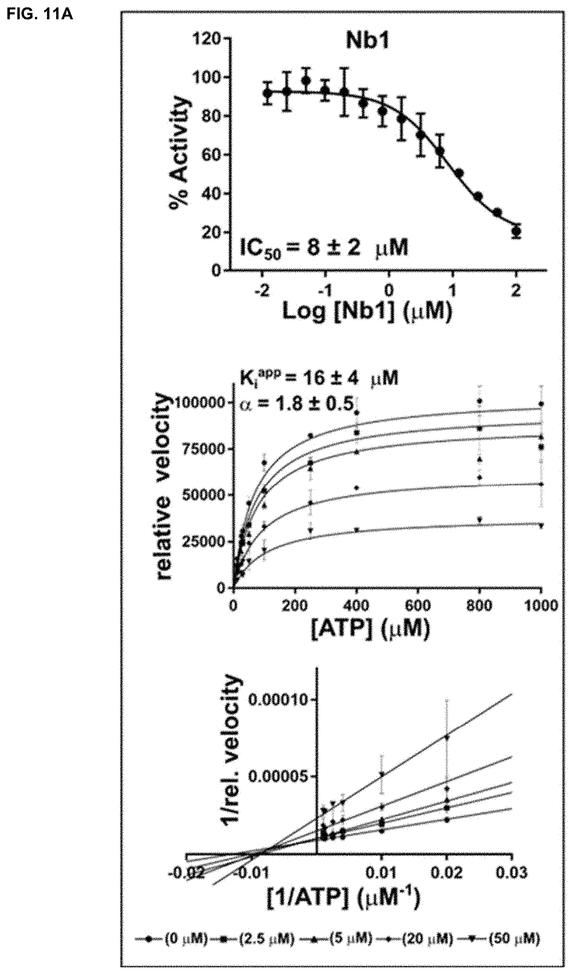

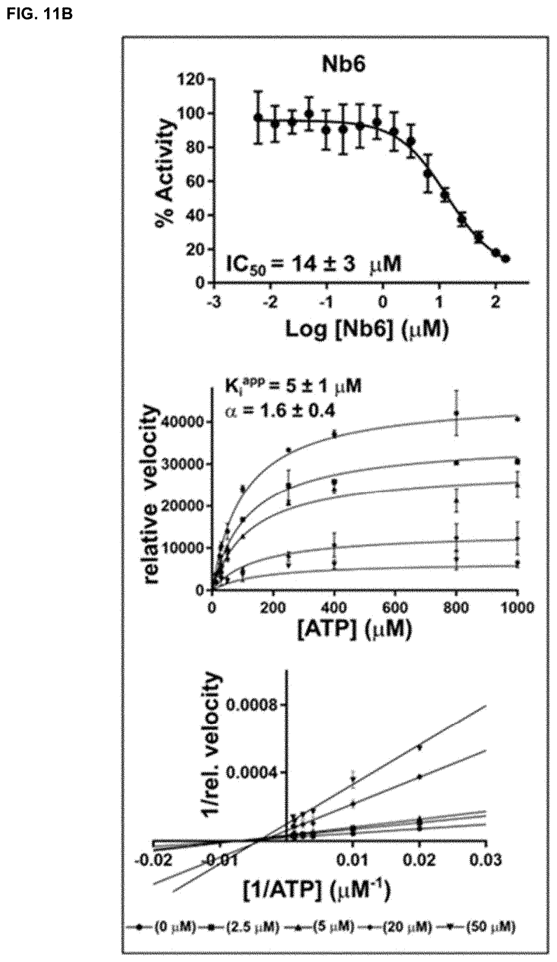

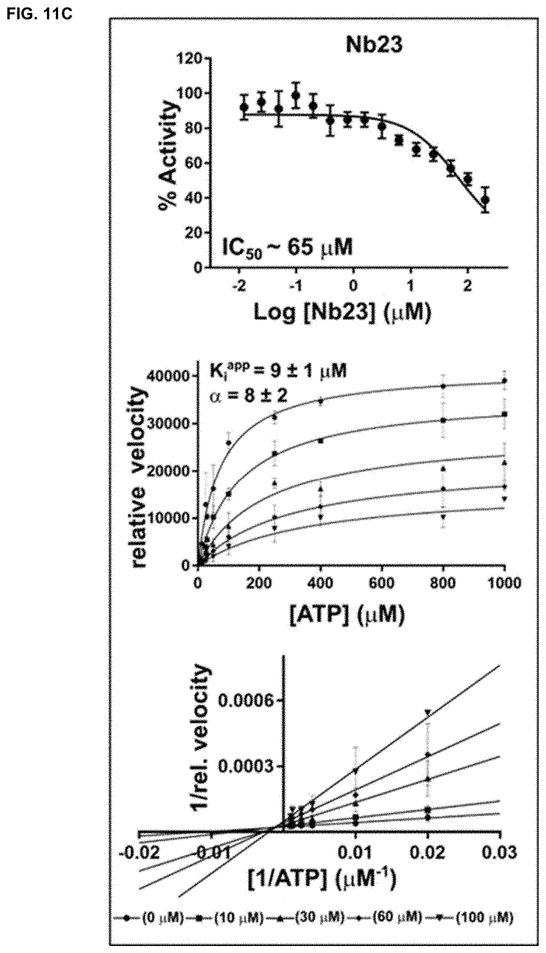

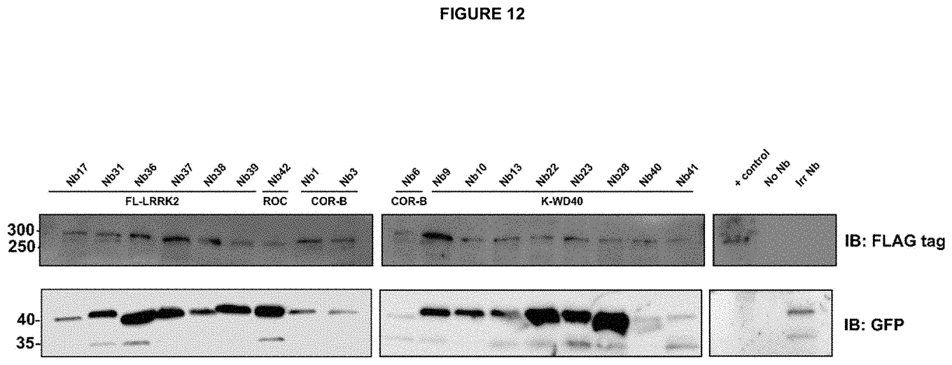

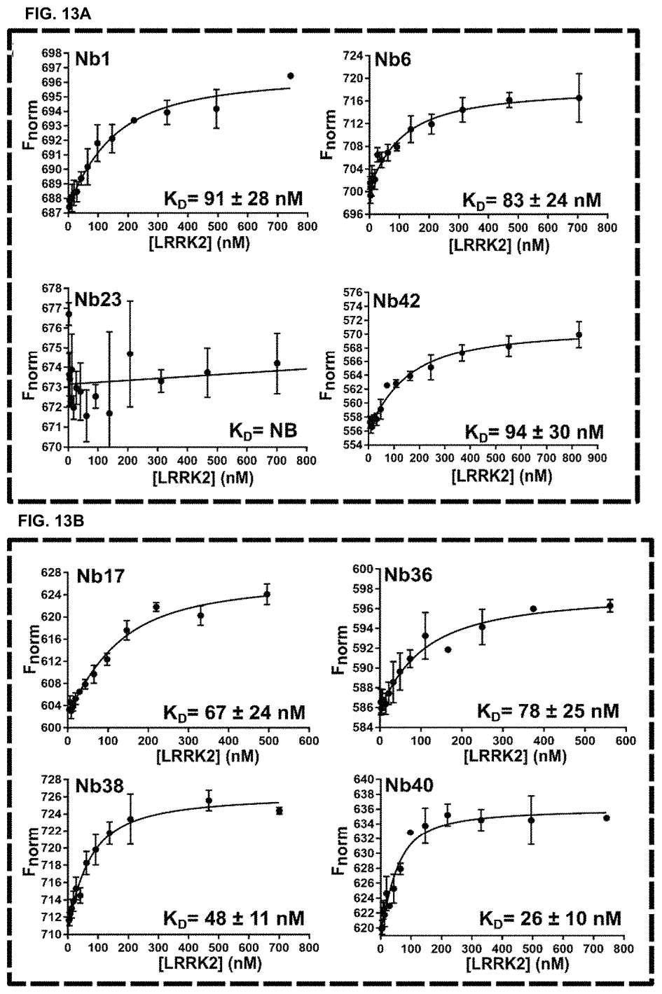

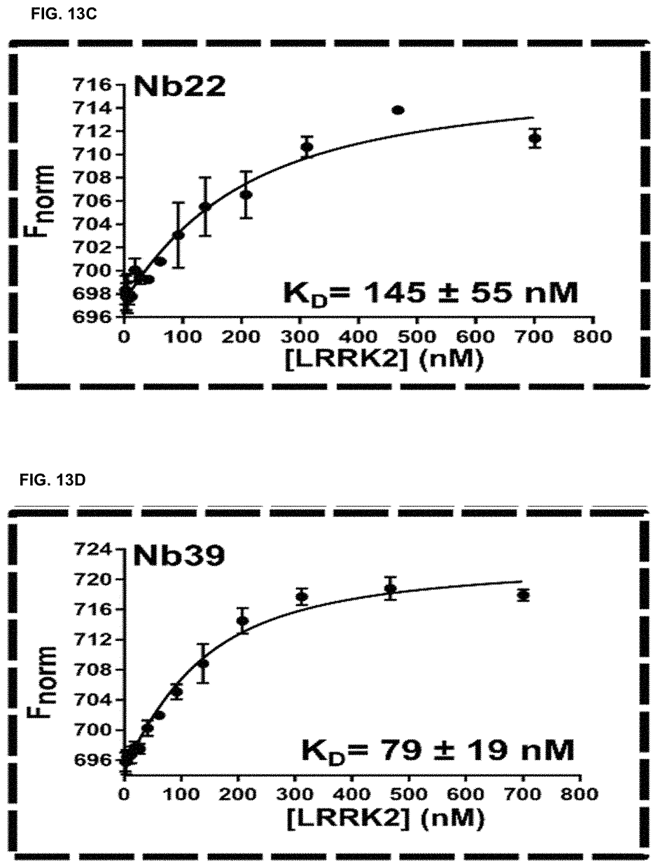

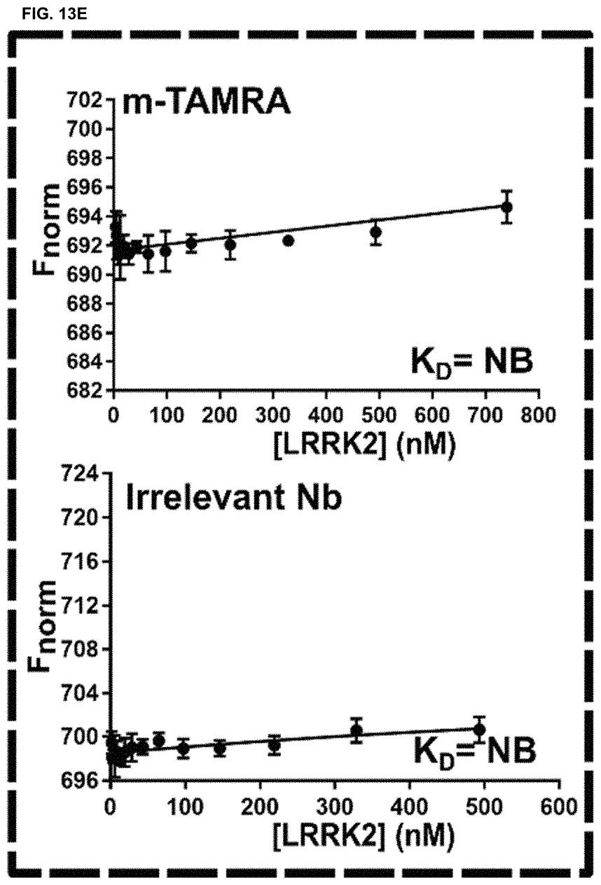

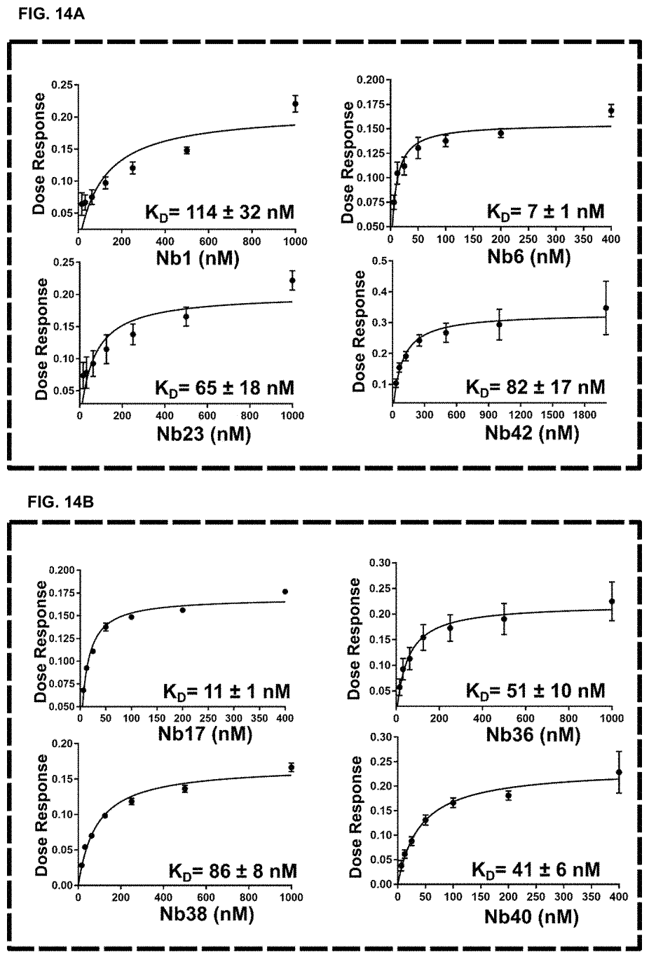

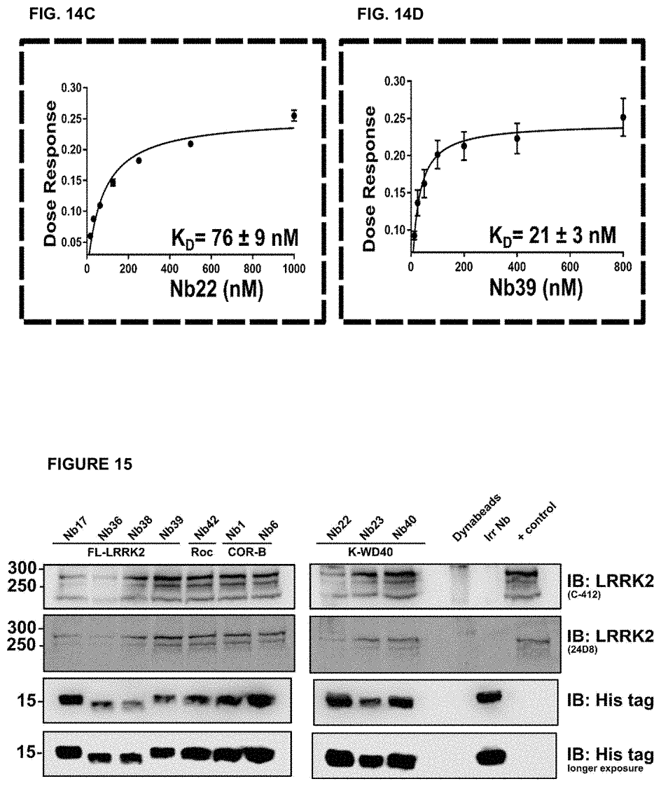

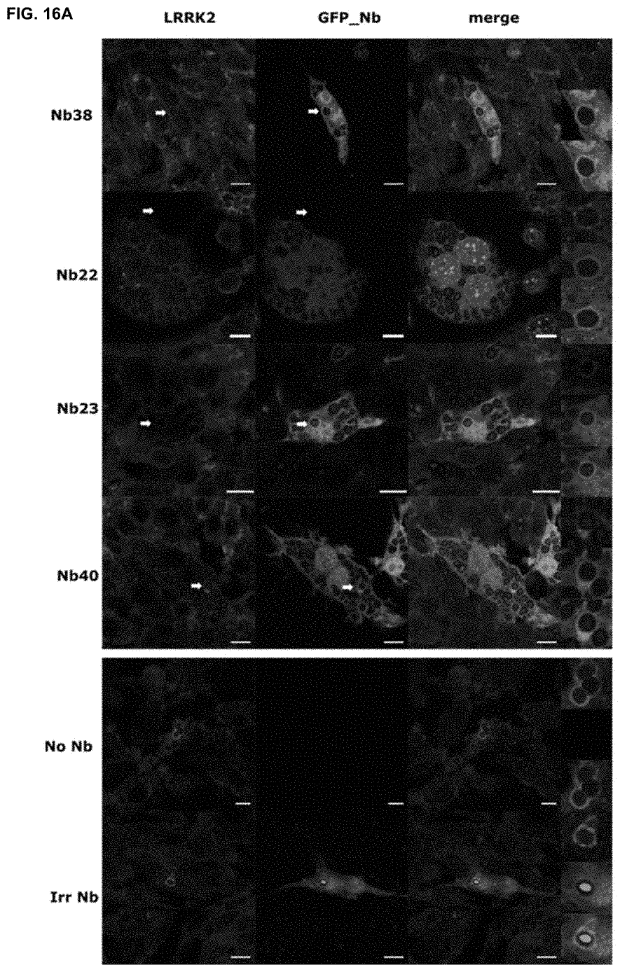

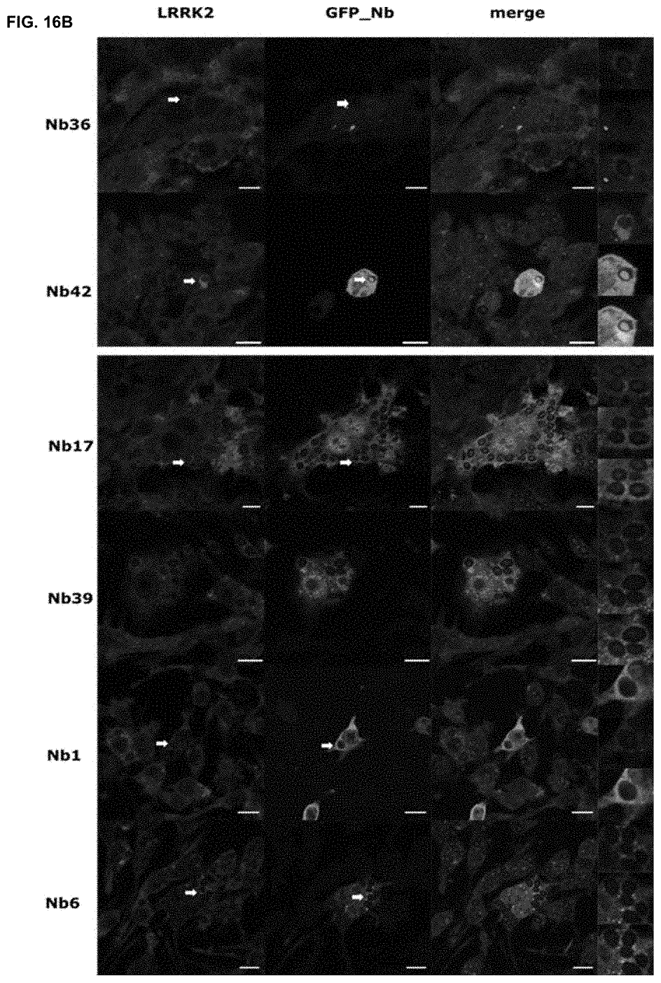

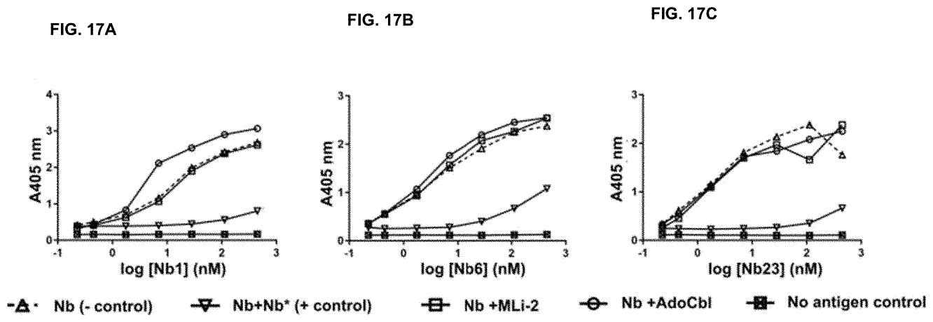

(FIGS. 2A-2C) LRRK2(G2019S) and its effector Rab29 were overexpressed together with GFP-fused Nbs (“Fluobody”) in HEK293T cells. A negative control, where no Nb is overexpressed (“No Nb”) is also included. Panel (FIG. 2A), (FIG. 2B) and (FIG. 2C) shows three repeats of the experiment. In rows labeled “pLRRK2”, LRRK2 pS1292 levels are determined by Western Blot using a site-specific anti-pLRRK2(pS1292) antibody (Abcam, ab203181) (shown at different times of development). In the rows labeled “pRAB10”, endogenous pT72-Rab10 levels are determined by Western Blot using the MJFF/Abcam antibody MJF-R21 (Abcam, ab230261) (shown at different times of development). The three lower rows contain controls of LRRK2 (rat anti-LRRK2, 24D8), Fluobody (rat anti-GFP) and Rab10 (rabbit anti-Rab10) expression levels. (FIG. 2D) Based on the results of panels (FIGS. 2A-2C) the Nbs can be divided into 4 functional groups, with group 1: Nbs that inhibit both LRRK2 autophosphorylation and Rab10 phosphorylation in cells; group 2: Nbs that inhibit only Rab10 phosphorylation; group 3: Nbs that activate LRRK2 kinase activity; group 4: Nbs that have no consistent/clear effect on LRRK2 kinase activity.

FIG. 3. Mapping of the binding epitopes of the Nbs on LRRK2 using cross-linking mass spectrometry. The Nbs are divided into 4 functional groups according to their effect on in cellulo LRRK2 kinase activity, as defined in FIG. 2d. The observed cross-links between the Nbs and LRRK2 are indicated by lines, with the corresponding lysine residues on LRRK2 indicated by their residue number. The domain specificity of the Nbs as determined previously in ELISA are given below the respective Nbs as a reference.

FIGS. 4A and 4B. Modulation of in vitro kinase activity by LRRK2-targeting Nbs. (FIG. 4A) Effect of selected LRRK2-targeting Nbs on LRRK2 kinase activity measured using the fluorescence-based PhosphoSens® Protein Kinase Assay, using the LRRK2-optimized AQT0615 peptide as substrate at a fixed concentration of 10 μM, and in the presence of 1 mM or 0.1 mM ATP, respectively. The influence of the different Nbs at a concentration of 25 μM on the relative kinase activity compared to the control where no Nb was added (“No Nb”) is plotted. Additional negative (25 μM of an irrelevant control Nb) and positive (25 μM of the ATP competitive LRRK2 inhibitor MLi-2) controls are included. (FIG. 4B) The influence of the different Nbs (25 μM) on the relative kinase activity compared to the “No-Nb” control is plotted, either in presence of 500 μM GDP (top) or 500 μM GTPγS (bottom). A positive control with 0.2 μM of the ATP-competitive LRRK2 inhibitor MLi-2 is included. Each bar reflects the average (±SD) of three independent measurements.

FIG. 5. Effect of LRRK2-targeting Nanobodies on cellular localization of LRRK2.

HEK 293 cells were co-transfected with the indicated GFP-Nbs and mScarlet-LRRK2. As a positive control mScarlet-LRRK2-transfected cells were treated with the pharmacological ATP-competitive inhibitor MLi-2 (1 μM MLi-2, 90 mins treatment), showing induction of LRRK2 relocalization onto microtubules seen as filamentous skein-like structures and indicated by white arrows (upper left panel). In contrast, cells co-transfected with GFP-Nbs and mScarlet-LRRK2 showed normal cytoplasmic distribution of LRRK2 with no relocalization to microtubules, similar to cells co-transfected with an irrelevant Nb. Scale bar 5 μm.

FIG. 6. Effect of LRRK2-targeting Nanobodies on MLi-2-induced microtubule relocalization of LRRK2. HEK 293 cells were co-transfected with the indicated GFP-Nbs and mScarlet-LRRK2 and treated with the pharmacological ATP-competitive inhibitor MLi-2 (1 μM MLi-2, 90 mins treatment). Co-transfection of mScarlett-LRRK2 with only GFP (upper left panel) or an irrelevant Nb (lower left panel) showed the Mli-2-induced relocalization of LRRK2 onto microtubules seen as filamentous skein-like structures and indicated by white arrows. Co-transfection with a subset of GFP-Nb inhibited the MLi-2-induced LRRK2 relocalization. Scale bar 5 μm.

FIG. 7. SDS-PAGE analysis showing purified Full-length and domain constructs of LRRK2.

3 μg of each purified full-length LRRK2 and the domain constructs, RocCOR, Roc, (MBP-)COR-B and kinase-WD40, as used in this study, was loaded on gel. Marker (left) Spectra™ Multicolor High Range Protein Ladder (Cat. No 26625; Thermo Scientific™); (right) PageRuler™ Prestained Protein Ladder, 10 to 180 kDa (Cat. No 26616; Thermo Scientific™) FIGS. 8A and 8B. LRRK2 Cross-linking.

Before immunization, LRRK2 was cross-linked with the lysine-specific cross-linker DSS. Two cross-link setups were performed, where cross-linking was allowed to proceed to different levels (FIGS. 8A and 8B). LRRK2 was purified in presence of GDP (lanes 2 and 3) or GTPγS (lanes 4 and 5). In both gels, Lane 2 and Lane 4 show LRRK2 before cross linking while Lane 3 and 5 show LRRK2 after DSS cross linking. For immunization 2, a mixture of the samples shown in lanes 5 was used, for immunization 3 a mixture of the samples shown in lanes 3 was used. Marker (FIG. 8A), Quad Color Protein Marker from Biozym; (FIG. 8B) Spectra™ Multicolor High Range Protein Ladder (Cat. No 26625 Thermo Scientific™)

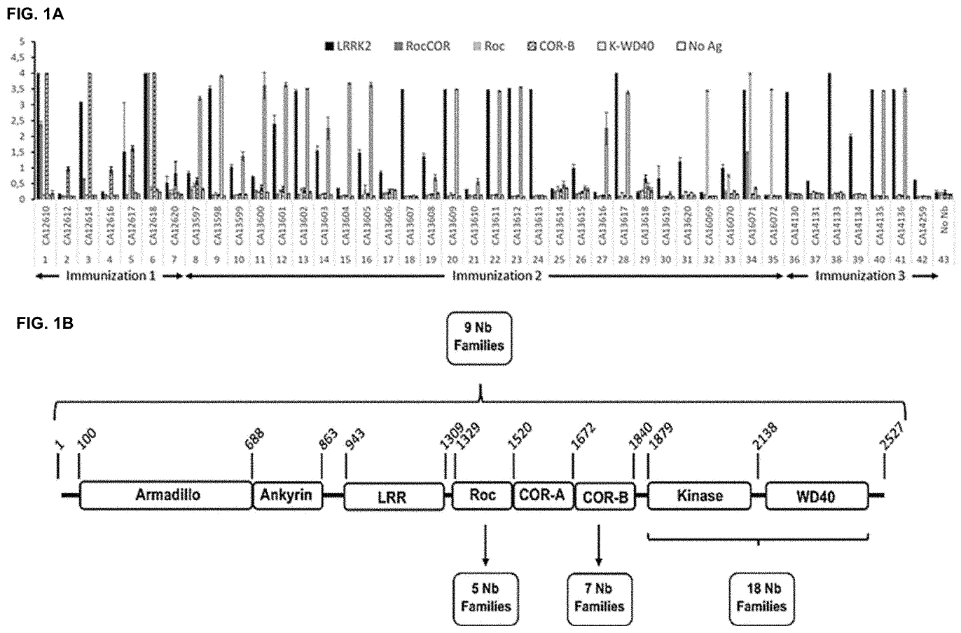

FIG. 9. SDS-PAGE of 42 purified Nanobodies, shown in FIG. 1, and the irrelevant Nb, used in this study.

2 μg of each nanobody was loaded on gel (except for CA16070 where 0.7 μg is loaded due to poor expression levels of this Nb). Marker, PageRuler™ Prestained Protein Ladder, 10 to 180 kDa (Cat. No 26616 Thermo Scientific™)

FIG. 10. Amino acid sequence of CA12610 Nb and CDR annotations.

Amino acid numbering according to Kabat. As an example of different CDR annotations possible for the Nbs disclosed herein, the regions corresponding to alternative CDR annotations (AbM, Chothia, Kabat, IMGT), as compared to the currently used one, are labelled in grey. Llama germline hallmark residues in bold/underlined.

FIGS. 11A-11C. Modulation of in vitro kinase activity by LRRK2-targeting Nbs.

FIGS. 11A-11C, Dose-response curves (upper panels) for the inhibition of the in vitro LRRK2 kinase activity by the group1 Nbs: Nb1 (FIG. 11A), Nb6 (FIG. 11B) and Nb23 (FIG. 11C), where the Nb concentration was varied from 200 or 150 μM to 0.006 μM in a two-fold serial dilution. The two lower panels show the Michaelis-Menten curves obtained for LRRK2 at varying concentrations of ATP and a fixed (sub-saturating) concentration of peptide substrate (AQT0615), and at varying concentrations of Nb1 (FIG. 11A), Nb6 (FIG. 11B) and Nb23 (FIG. 11C), and the corresponding linearizations according to the Lineweaver-Burk method (double-reciprocal plot). The Nb concentrations used are indicated below the plots. Each datapoint reflects the average (±SD) of three independent measurements. The IC₅₀ (±SD) values resulting from fitting on a three-parameter logistic equation and the Kᵢ^{app} and α values (±SD) resulting from global fitting on a mixed-type inhibition mechanism are indicated on the graphs.

FIG. 12. Nanobodies bind and immune-precipitate LRRK2.

HEK293 cells were transiently co-transfected with GFP-tagged Nbs and (S)trep-(F)lag-tagged LRRK2 constructs for 48 hours prior to lysis. Pulldown assays were performed by means of magnetic GFP-Trap beads. HEK293 cell lysate only overexpressing SF-LRRK2 was used as a negative control. Nbs and LRRK2 were detected via immunoblotting. Unlike an irrelevant Nb and the negative control, all tested Nbs pull-down LRRK2. Blot is representative of n=3.

FIGS. 13A-13E. Affinity measurements of the Nbs for LRRK2 using Microscale thermophoresis (MST).

Binding isotherms are shown, obtained by titrating increasing concentrations of LRRK2 to fluorescently (m-TAMRA)-labeled Nbs and measuring the MST signals. Nbs are classified into four functional groups as defined in FIG. 2d with (FIG. 13A) group 1 Nbs, (FIG. 13B) group 2 Nbs, (FIG. 13C) group 3 Nbs, and (FIG. 13D) group 4 Nbs. In panel (FIG. 13E) two negative controls are shown, where LRRK2 was either titrated to free m-TAMRA or to a m-TAMRA-labeled irrelevant Nb. All measurements were performed in presence of 500 μM GDP, except for Nb42 where GTPγS was used. The corresponding equilibrium dissociation constants (K_{d}±standard error) obtained by fitting with a quadratic binding equation are given (each data point is the average of three independent measurements with the error bars representing the standard deviation; NB=no MST binding signal detectable).

FIGS. 14A-14D. Affinity measurements of the Nbs for LRRK2 using Biolayer Interferometry (BLI).

Binding isotherms are shown, obtained by titrating increasing concentrations of Nbs to LRRK2 that was trapped on a Streptavidine biosensor via biotinylated Nb40 (or Nb42 in case of affinity measurement of Nb40) and measuring the BLI signal. Nbs are classified into four functional groups as defined in FIG. 2d with (FIG. 14A) group 1 Nbs, (FIG. 14B) group 2 Nbs, (FIG. 14C) group 3 Nbs, and (FIG. 14D) group 4 Nbs. All measurements were performed in presence of 500 μM GDP. The corresponding equilibrium dissociation constants (K_{d}±standard error) obtained by fitting with a Langmuir binding equation are given (each data point is the average of three independent measurements with the error bars representing the standard deviation).

FIG. 15. Nanobodies bind and immune-precipitate endogenous LRRK2.

Lysates derived from RAW264.7 cells were incubated with 1.5 μM of purified His-tagged Nbs and pulldowns were performed using magnetic Dynabeads. LRRK2 was detected via immunoblotting, demonstrating that all tested Nbs immune-precipitate LRRK2. Blot is representative of n=3.

FIGS. 16A and 16B. Nanobodies colocalize with endogenous LRRK2.

RAW264.7 cells were transfected with GFP-fused Nbs and treated with Zymosan for 30 mins. Recruitment of LRRK2 and Nbs to the phagosomes was analyzed by immunofluorescence. FIG. 16A, images for Nb38, Nb22, Nb23, Nb40, No Nb, or Irrelevant (IRR) Nb treatments; FIG. 16B, images for Nb36, Nb42, Nb17, Nb39, Nb1, and Nb6 treatments. Scale bar=10 μm.

FIGS. 17A-17C. Nanobodies bind LRRK2 through binding sites differing from previously described LRRK2 kinase inhibitors.

Results of the competition ELISA titration experiments assessing whether the ATP competitive kinase inhibitor Mli-2 and the previously described non-ATP competitive LRRK2 inhibitor 5′deoxyadenosylcobalamin (AdoCbl=coenzyme B₁₂) compete for the same binding sites as the group 1 Nanobodies: Nb1 (FIG. 17A), Nb6 (FIG. 17B) and Nb23 (FIG. 17C). LRRK2 was coated in the wells of the ELISA plate and the ELISA signal for a dilution series of the respective Nbs (detected via their C-terminal EPEA-tag) is plotted in function of the Nb concentration. The effect of the presence of a large excess of MLi-2 (1 M), AdoCbl (250 μm) and the corresponding untagged Nb as a positive (+) control (Nb*, at 9 μM) is determined. A “no antigen control”, where no LRRK2 was coated on the bottom of the well, is also included. In contrast to the positive control where addition of Nb* causes a clear rightward shift of the ELISA titration curves compared to the titration curve of the Nb alone (“- control”), neither Mli-2 nor AdoCbl show a rightward shift of the curves.

### DETAILED DESCRIPTION

The present invention will be described with respect to particular embodiments and with reference to certain drawings, but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. Affinity is the strength of binding of a single molecule to its ligand. It is typically measured and reported by the equilibrium dissociation constant (K_{D}), which is used to evaluate and rank order strengths of bimolecular interactions. The binding of an antibody to its antigen is a reversible process, and the rate of the binding reaction is proportional to the concentrations of the reactants. At equilibrium, the rate of [antibody] [antigen] complex formation is equal to the rate of dissociation into its components [antibody]+[antigen]. The measurement of the reaction rate constants can be used to define an equilibrium or affinity constant (1/K_{D}). In short, the smaller the K_{D} value the greater the affinity of the antibody for its target. The rate constants of both directions of the reaction are termed: the association reaction rate constant (Kₒₙ), which is the part of the reaction used to calculate the “on-rate” (Kₒₙ), a constant used to characterize how quickly the antibody binds to its target. Vice versa, the dissociation reaction rate constant (K_{off}), is the part of the reaction used to calculate the “off-rate” (K_{off}), a constant used to characterize how quickly an antibody dissociates from its target. In measurements as shown herein, the flatter the slope, the slower off-rate, or the stronger antibody binding. Vice versa, the steeper downside indicates a faster off-rate and weaker antibody binding. The ratio of the experimentally measured off- and on-rates (K_{off}/Kₒₙ) is used to calculate the K_{D} value. Several determination methods are known to the skilled person to measure on and off rates and to thereof calculate the K_{D} (see below and examples), which is therefore, taking into account standard errors, considered as a value that is independent of the assay used. As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions.

A “binding agent” relates to a molecule that is capable of binding to another molecule, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. The term “binding pocket” or “binding site” refers to a region of a molecule or molecular complex, that, as a result of its shape and charge, favourably associates with another chemical entity, compound, proteins, peptide, antibody or Nb. The term “pocket” includes, but is not limited to cleft, channel or site. The term “part of a binding pocket/site” refers to less than all of the amino acid residues that define the binding pocket, or binding site. For example, the portion of residues may be key residues that play a role in ligand binding or may be residues that are spatially related and define a three-dimensional compartment of the binding pocket. The residues may be contiguous or non-contiguous in primary sequence. For antibody-related molecules, the term “epitope” is also used to describe the binding site, as used interchangeably herein. An “epitope”, refers to an antigenic determinant of a polypeptide, constituting a binding site or binding pocket on a target molecule, such as the LRRK2 protein, more specifically a binding pocket on the LRRK2 domains accessible for the ISVDs or VHHs. An epitope could comprise 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. The term “conformation” or “conformational state” of a protein refers generally to the range of structures that a protein may adopt at any instant in time. A conformational epitope may thus comprise amino acid interactions from different protein domains of the LRRK2 protein. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody”, “antibody fragment” and “active antibody fragment” as used herein refer to a protein comprising an immunoglobulin (Ig) domain or an antigen binding domain capable of specifically binding the antigen, in this case the LRRK2 protein. ‘Antibodies’ can further be intact immunoglobulins derived from natural sources or from recombinant sources and can be immunoreactive portions of intact immunoglobulins. Antibodies are typically tetramers of immunoglobulin molecules. The term “active antibody fragment” refers to a portion of any antibody or antibody-like structure that by itself has high affinity for an antigenic determinant, or epitope, and contains one or more complementarity-determining-regions (CDRs) accounting for such specificity. Non-limiting examples include immunoglobulin domains, Fab, F(ab)′2, scFv, heavy-light chain dimers, immunoglobulin single variable domains, Nanobodies, domain antibodies, and single chain structures, such as a complete light chain or complete heavy chain. An additional requirement for “activity” of said fragments in the light of the present invention is that said fragments are capable of binding LRRK2, and preferably are allosteric modulators of LRRK2, more preferably capable to increase or decrease LRRK2 activity in a subject. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulfide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention also refers to “immunoglobulin single variable domains” (abbreviated as “ISVD”), equivalent to the term “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

In particular, the immunoglobulin single variable domain may be a Nanobody® (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens.

As used herein, the terms “determining,” “measuring,” “assessing,”, “identifying”, “screening”, and “assaying” are used interchangeably and include both quantitative and qualitative determinations.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a lama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present. The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

### DETAILED DESCRIPTION

The present invention relates to non-naturally occurring proteinaceous binding agents of human LRRK2 protein, specifically allosteric modulators of LRRK2 activity. Mutations in the Parkinson's disease (PD)-associated protein leucine-rich repeat kinase 2 (LRRK2) commonly lead to a reduction of GTPase activity and increase in kinase activity. LRRK2 (dominant) mutations, with G2019S being the most prevalent one, cause late-onset Parkinson's disease, mediated via increased phosphorylation of Rab proteins. So, inhibition of kinase activity, and/or increasing of GTPase activity provide for a therapeutically beneficial outcome. Indeed, known ATP-competitive inhibitors of LRRK2 kinase activity have demonstrated potential therapeutic benefits in PD treatment, but concerns regarding potential detrimental side effects associated with the use of kinase inhibitors directed toward the LRRK2 ATP-binding pocket remain [46, 81-82]. While further generation compounds acting in an orthosteric mechanism are in clinical testing phases, a need for compounds with a better safety profile requires alternative approaches, with allosteric modulation of LRRK2 activity as one of the novel approaches and in scope of the present disclosure.

The present invention relates to allosteric binding agents that predominantly bind LRRK2 via conformational epitopes at domains different from the ATP-catalytic binding site in the kinase domain, and/or not directly competing with ATP for its active site, neither competing with known binding agents of LRRK2, or specifically of the LRRK2 kinase domain. The spectrum of binding agents that is described herein encompasses several layers of allosteric modulation of LRRK2, which will support the creation of novel first-in-class LRRK2 therapeutics. In particular, the binding agents of the present invention all efficiently bind LRRK2 from cell lysates, both upon LRRK2 over-expression and at endogenous LRRK2 expression levels, which reflects a high affinity binding, as confirmed via two independent biophysical methods which yielded K_{D} values ranging from 10 to 200 nM. Further affinity maturation, generation of multiparatopic constructs and/or humanization may even increase this human LRRK2 protein binding affinity to subnanomolar affinities.

Furthermore, the LRRK2-specific immunoglobulin-single-variable domains (ISVDs) provide for a first proteinaceous LRRK2 binder acting through an allosteric mechanism and acting on LRRK2 activity via a mixed (non-competitive) type inhibition mechanism. Similar to four out of the five common PD mutations, treatment with Type 1 ATP-competitive inhibitors has been shown to cause LRRK2 microtubule association. This phenomenon of oligomerization of LRRK2 on microtubules, with concomitant blocking of microtubule-associated motor proteins, has been suggested as one of the underlying causes of LRRK2 pathology [36]. Surprisingly, none of the ISVD-based LRRK2-specific binders described herein induce LRRK2 association with microtubules, while some even revert the Type I ATP-competitive kinase inhibitor MLI-2-induced LRRK2 relocalization. These observations provide for a differentiating effect of the LRRK2 binders of the present invention, positioning them as candidates for further development into LRRK2 modulators with a different mode of action and cellular profile to the currently existing inhibitors.

So the present invention provides for allosteric LRRK2 protein binding agents, which bind human LRRK2 in cells without affecting the protein its subcellular localisation, preserving its cellular distribution and thus retain LRRK2 detached from or not associated with microtubules. So in one embodiment, the LRRK2-specific binding agents or modulators described herein do not induce LRRK2 accumulation on microtubules. Furthermore, the ISVD-based LRRK2 allosteric binding agents as disclosed herein provide for a binding affinity, as may be determined using methods as known by the skilled person, or as exemplified herein, which relate to a K_{D} value as defined herein of 500 nM or lower, preferably 200 nM or lower, more preferably between 150 nM and values that are 10-fold lower, or 100-fold lower or 1,000-fold lower or 10,000-fold lower.

More specifically, the binding agents allosterically modulate LRRK2 kinase activity, most preferably in cells and/or in vitro. The modulation of kinase activity may be linked to the monomer/dimerization cycle of LRRK2 as well as to the GTPase activity of the protein, although the LRRK2 modulators as described herein focus on the one hand on the inherent advantageous effect of applying ISVDs to target LRRK2, and on the other hand on their effect obtained by high affinity binding in a conformational manner, which results, depending on the binding position, in kinase activity modulation.

So in a specific aspect, the LRRK2 allosteric binding agents comprise immunoglobulin single variable domains (ISVDs) or VHHs or Nanobodies, as used interchangeably herein, which bind conformational epitopes to modulate the LRRK2 protein conformation and which may thereby affect its activity. Their effect has been analyzed in cellulo for a wider panel of 18 different ISVD families. These ISVDs were classified according to their modulating profile (LRRK2 subcellular localisation, kinase activity inhibition or activation) and binding mode (different conformational epitopes and binding sites), providing for a common feature of in cellulo allosteric modulation of LRRK2. Moreover, their interaction with LRRK2 is identified to be predominantly located outside the kinase domain, diversifying these highly specific proteinaceous binding agents from all known ATP-competitive kinase inhibitors and even from the natural compound 5′-deoxyadenosylcobalamin (AdoCbl; a VitaminB12 physiological form; [43]; as shown in Example 10). In one embodiment, the invention relates to an allosteric modulator of human LRRK2, specifically binding LRRK2 protein with high affinity, wherein said modulator is different from, or not comprising, a naturally occurring LRRK2 modulator or binder, such as Vitamin B12, a physiological form of cobalamin, or derivatives thereof.

Moreover, in contrast to conventional LRRK2-specific antibodies, the LRRK2 modulating ISVDs of the present invention are small agents and therefore may as well be applied to study the dynamic localization of endogenous LRRK2 in living cells (in vivo imaging).

The present invention hence for the first time reveals highly specific LRRK2 binding agents that allosterically modulate its activity via a mechanism that is capable of avoiding further unfavourable effects such as relocalization of the LRRK2 protein in the cell. Such allosteric binders may pronounce activating or inhibiting effects on LRRK2, for which currently kinase inhibitors are considered therapeutically most relevant. The only reported small compound inhibiting in an allosteric manner is a Vitamin B12 derivative [43], though its binding site is predominantly located in the LRRK2 kinase domain, thereby providing for an at least partially different binding site and different mechanism of action as compared to the allosteric ISVDs as disclosed herein. Furthermore, the ISVDs have been demonstrated to specifically bind to the Roc domain, to the COR domain, to the kinase domain, to the WD40 domain and/or on the interface of various LRRK2 domains, including the N-terminal (armadillo, ankyrin repeat, LRR) domains and kinase or WD40 domains. Lacking a high-resolution structure of full-length LRRK2, the precise epitope binding sites are not yet established, though insights from ELISA and cross-linking mass spectrometry data provided the indicative region where the allosteric ISVDs are binding with said cross-link between the proteins at a distance of maximally 35 angstrom. From this information, it was concluded that a large fraction of the LRRK2 allosteric modulators as described herein function via binding to a conformational epitope that is predominantly located outside of the kinase domain, and/or has its epitope binding site residues at least located outside the kinase active site.

The allosteric modulators as described herein are ‘non-natural’, ‘non-naturally-occurring’ or ‘unnatural’ binding agents, as interchangeably used herein, which refers to the fact that these binding agents or modulators do no occur in nature as such, i.e. a technical step or process is required, such as immunization, to obtain such highly specific allosteric modulator binding agents. In contrast, the reported Vitamin B12 derivative compound binding the kinase domain of LRRK2 is known to exist in nature as Cobalamin in four forms. Cobalamin actually refers to a group of complex, chemically related co-factors that require cobalt (Co) for function. Hydroxycobalamin is produced by bacteria and cyanocobalamin (CNCbl) is a form derived during the purification of hydroxycobalamin (OHCbl) for therapeutic or supplementation purposes. Both are further metabolized in the body to form the active forms, adenosylcobalamin (AdoCbl) and methylcobalamin (MeCbl).

The term “allosteric modulation”, “allosteric activity” or “allosteric regulation”, as used herein, refers to binding of said agent at an allosteric or regulatory site, which is a site different from the enzymatically active site or catalytic site of the protein, so different to the binding site of orthosteric binders. Such catalytic sites of LRRK2 comprise the kinase domain active site as well as the GTPase domain active site. “Modulators” are either positive, negative or neutral. “Positive allosteric modulators” increase the activity or response of the LRRK2 by either increasing the probability that an agonist or ligand, such as ATP or GTP, will bind to LRRK2 (i.e. increase affinity), or increasing its ability to activate LRRK2 (i.e. increasing efficacy), or both. “Negative allosteric modulators” decrease the agonist affinity and/or efficacy. “Neutral allosteric modulators” do not affect agonist activity but can stop other modulators from binding to an allosteric site. Some modulators may also work as allosteric agonists. The allosteric binding agent may for instance affect or modulate the LRRK2 activity, through the induction of a conformational change of the LRRK2 protein upon binding. In one embodiment, said allosteric inhibitor of LRRK2 activity is a non-naturally occurring molecule, and is therefore different from naturally present compounds such as Cobalamin derivatives in (metabolizing) humans. So, in a specific embodiment, said LRRK2 allosteric modulator is a LRRK2 inhibitor which is not cobalamin or a cobalamin derivative. The term ‘derivative’ as used herein includes, but is not limited to the cobalamin derivative AdoCbl, MeCbl, OHCbl, and CNCbl, and/or any cobalamin-scaffold containing natural compound. Preferably, said LRRK2 allosteric modulator specifically binding LRRK2 is a heterozygous or exogenous compound when present in a cell, an organism, or a subject.

The present invention specifically relates to allosteric modulators of human LRRK2. It is clear for the skilled person that bacterially-originating LRRK2 protein is quite diversified as compared to the human LRRK2 protein (SEQ ID NO: 21), not only from the primary structure or amino acid sequence, but also from secondary and tertiary structure, for instance in the fact that bacterial LRRK2 lacks a kinase domain. Human LRRK2 is a complex and larger protein, implying an immaculate design to generate specific binders capable to strongly affect human LRRK2 activity. The binding agents or ISVDs and allosteric modulators as described herein were obtained via a well-defined immunization and selection strategy, and provide for novel binding agents with novel conformation epitopes of high therapeutic potential. ISVDs or more specifically Nanobodies are known to act as stabilizers or chaperones in structural biology analysis, and moreover have been developed into therapeutics as well. Targeting human LRRK2 with an allosteric ISVD or Nb or an active antibody fragment derived thereof has not been shown previously, and although their therapeutic potential may be more complex in view of intracellular targeting of LRRK2 and the hurdle of reaching the brain for PD treatment, their binding modes provide for several unique approaches of producing and selecting novel compounds to improve LRRK2 drugs. In fact, their conformational epitopes provide for novel druggable pockets, and in addition their high specificity and allosteric effect may in combination with their subtle mode of action with retained LRRK2 cellular localisation probably result in reduced toxicity risks. With respect to drug delivery, their small size is advantageous to aide in crossing the blood-brain-barrier, or to link them to cargos or capturing within vehicles crossing the BBB, as further described herein. Finally, the Nanobodies may also be therapeutically applied as intrabodies, and may be applied using gene therapy.

In one embodiment, said LRRK2 allosteric modulators comprise binding agents specifically and predominantly binding the Roc domain of LRRK2. In another embodiment, said LRRK2 allosteric modulators comprise binding agents specifically and predominantly binding the COR(-B) domain of LRRK2. In another embodiment, said LRRK2 allosteric modulators comprise binding agents specifically and predominantly binding the WD40 domain of LRRK2. In another embodiment, said LRRK2 allosteric modulators comprise binding agents specifically and predominantly binding the kinase domain of LRRK2. In another embodiment, said LRRK2 allosteric modulators comprise binding agents specifically binding the interface of various LRRK2 domains, meaning binding residues on several domains, and may include the N-terminal (armadillo, ankyrin repeat, LRR) domains and kinase or WD40 domains. In some embodiments, said LRRK2 allosteric modulators specifically bind a combination of residues present on any of said domains. In a preferred embodiment, the LRRK2 allosteric modulators as described herein do not bind the active site of the kinase or GTPase domain of LRRK2. In a more preferred embodiment, the LRRK2 allosteric modulators as described herein do not bind the kinase domain amino acid residues, and specifically bind a LRRK2 binding site comprising amino acid residues belonging to other protein domains of LRRK2.

In another embodiment, said allosteric modulator specifically binding LRRK2 protein may increase its kinase and/or decrease its GTPase activity. In other embodiments, the allosteric modulator specifically binding LRRK2 protein may decrease, inhibit or block its kinase and/or increase its GTPase activity. The term ‘increase’, ‘enhance’ or ‘activate’ is used interchangeably herein and refers to an increment of at least 5% of its activity as compared to a control without the allosteric modulator or with a negative or irrelevant control agent. The term ‘increase’, ‘enhance’, or ‘activate’ further refers to an increment of at least 10%, 15%, 20%, 25%, 30%, 40%, 50%, or more than 50% of its activity as compared to a control without the allosteric modulator or with a negative irrelevant vehicle control. The term ‘decreased’, ‘reduced’ or ‘inhibition’ or ‘to prevent’ as used interchangeably herein refers to a reduction of at least 5% of its activity as compared to a control without the allosteric modulator or with a negative or irrelevant control. The term ‘decreased’, ‘reduced’, ‘prevent’ or ‘inhibit’ further refers to a reduction of at least 10%, 15%, 20%, 25%, 30%, 40%, 50%, or more than 50% of its activity as compared to a control without the allosteric modulator or with a negative control. The term ‘block’ of LRRK2 activity refers to a reduction of its activity to a non-detectable level as compared to a control without the allosteric modulator or with a negative control. A ‘negative control’ or ‘irrelevant control’ or ‘control’ or ‘vehicle control’ as referred to herein is meant a binding agent of similar nature (e.g. an irrelevant Nb) that is not bound to LRRK2, or that is bound to LRRK2 but known not to have any effect on its activity. A ‘control’ may be one type of molecule or a pool of molecules known to have no effect on LRRK2 (irrelevant Nbs or compounds, etc.).

When referring to an inhibition of kinase activity of LRRK2, it is meant herein that its capacity to autophosphorylate and/or to phosphorylate its substrates is reduced. Vice versa, when referring to activation or increase of kinase activity of LRRK2 as described herein, it is meant that LRRK2 is affected by the allosteric modulators to result in an increase in autophosphorylation and/or substrate phosphorylation. A substrate as referred to herein includes but is not limited to the Rab proteins referred to in the Examples, but also includes the peptide as used in commercial in vitro assays. Autophosphorylation has been reported at several LRRK2 amino acid positions, as known to the skilled person from the state of the art.

In another embodiment, said allosteric modulator of LRRK2 protein activity comprises a compound, a chemical, a protein, a peptide or peptidomimetic, an antibody, antibody mimetic, single domain antibody ISVD or any active antibody fragment. The term “compound” as used herein describes any molecule, either naturally occurring or synthetic that is designed, identified, screened for, or generated and may be tested in an assay, such as a screening assay or drug discovery assay, or specifically in a method for identifying a compound capable of modulating LRRK2 activity. As such, these compounds comprise organic and inorganic compounds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules” or “small compounds”, which refers to a low molecular weight (e.g., <900 Da or <500 Da) organic compound. The allosteric modulators also include chemicals, and compounds such as polynucleotides, lipids or hormone analogs that are characterized by low molecular weights. Other biopolymeric organic test compounds include small peptides or peptide-like molecules, or derivatives thereof, such as a peptidomimetic containing synthetic amino acids (peptidomimetics) comprising from about 2 to about 40 amino acids.

Compounds of the present invention include both those designed or identified using a screening method and those which are capable of conformationally binding LRRK2, as specifically defined herein for the ISVDs of the present invention. Such compounds may also be produced using a screening method based on use of the structural conformations obtained for LRRK2 in complex with the ISVDS of the invention as presented herein. The candidate compounds and/or compounds identified or designed using a method of the present invention may be any suitable compound, synthetic or naturally occurring, preferably synthetic. In one embodiment, a synthetic compound selected or designed by the methods of the invention preferably has a molecular weight equal to or less than about 5000, 4000, 3000, 2000, 1000 or more preferably less than about 500 Da, or is preferably a peptide. A compound of the present invention is preferably soluble under physiological conditions. Such compounds can comprise functional groups necessary for structural interaction with proteins, particularly hydrogen bonding, and typically include at least an amine, carbonyl, hydroxyl or carboxyl group, preferably at least two of the functional chemical groups. The compound may comprise cyclical carbon or heterocyclic structures and/or aromatic or polyaromatic structures substituted with one or more of the above functional groups. Compounds can also comprise biomolecules including peptides, saccharides, fatty acids, steroids, purines, pyrimidines, derivatives, structural analogues, or combinations thereof. Compounds may include, for example: (1) peptides such as soluble peptides, or peptidomimetics, including Ig-tailed fusion peptides and members of random peptide libraries and combinatorial chemistry-derived molecular libraries made of D- and/or L-configuration amino acids, and/or conformationally restrained amino acid derivatives; (2) phosphopeptides (e.g. members of random and partially degenerate, directed phosphopeptide libraries, (3) antibodies (e.g., polyclonal, monoclonal, humanized, anti-idiotypic, chimeric, and single chain antibodies, nanobodies as well as Fab, (Fab)₂, Fab expression library and epitope-binding fragments of antibodies); (4) non-immunoglobulin binding proteins such as but not restricted to avimers, DARPins and lipocalins; (5) nucleic acid-based aptamers; and (6) small organic and inorganic molecules.

Synthetic compound libraries are commercially available from, for example, Maybridge Chemical Co. (Tintagel, Cornwall, UK), AMRI (Budapest, Hungary) and ChemDiv (San Diego, Calif.), Specs (Delft, The Netherlands), ZINC15 (Univ. of California). In addition, numerous means are available for random and directed synthesis of a wide variety of organic compounds and biomolecules, including expression of randomized oligonucleotides. Alternatively, libraries of natural compounds in the form of bacterial, fungal, plant and animal extracts can be readily produced. In addition, natural or synthetic compound libraries and compounds can be readily modified through conventional chemical, physical and biochemical means and may be used to produce combinatorial libraries. In addition, numerous methods of producing combinatorial libraries are known in the art, including those involving biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide or peptide libraries, while the other four approaches are applicable to polypeptide, peptide, nonpeptide oligomer, or small molecule libraries of compounds. Compounds also include those that may be synthesized from leads generated by fragment-based drug design, wherein the binding of such chemical fragments is assessed by soaking or co-crystallizing such screen fragments into crystals provided by the invention and then subjecting these to an X-ray beam and obtaining diffraction data.

Further, compounds identified or designed using the methods of the invention can be a peptide or a mimetic thereof. The isolated peptides or mimetics of the invention may be conformationally constrained molecules or alternatively molecules which are not conformationally constrained such as, for example, non-constrained peptide sequences. The term “conformationally constrained molecules” means conformationally constrained peptides and conformationally constrained peptide analogues and derivatives. In addition, the amino acids may be replaced with a variety of uncoded or modified amino acids such as the corresponding D-amino acid or N-methyl amino acid. Other modifications include substitution of hydroxyl, thiol, amino and carboxyl functional groups with chemically similar groups. With regard to peptides and mimetics thereof, still other examples of other unnatural amino acids or chemical amino acid analogues/derivatives can be introduced as a substitution or addition. Also, a peptidomimetic may be used. A peptidomimetic is a molecule that mimics the biological activity of a peptide but is no longer peptidic in chemical nature. By strict definition, a peptidomimetic is a molecule that no longer contains any peptide bonds (that is, amide bonds between amino acids). However, the term peptide mimetic is sometimes used to describe molecules that are no longer completely peptidic in nature, such as pseudo-peptides, semi-peptides and peptoids. Whether completely or partially non-peptide, peptidomimetics for use in the invention, provide a spatial arrangement of reactive chemical moieties that closely resembles the three-dimensional arrangement of active groups in the peptide on which the peptidomimetic is based. For instance a peptide or peptidomimetic may be designed as to mimic the paratopes or CDRs of the ISVDs described herein. Typically, as a result of this similar active-site geometry, peptidomimetics has effects on biological systems which are similar to the biological activity of the peptide. There are sometimes advantages for using a mimetic of a given peptide rather than the peptide itself, because peptides commonly exhibit two undesirable properties: (1) poor bioavailability; and (2) short duration of action. Peptide mimetics offer an obvious route around these two major obstacles, since the molecules concerned are small enough to be both orally active and have a long duration of action. There are also considerable cost savings and improved patient compliance associated with peptide mimetics, since they can be administered orally compared with parenteral administration for peptides. Furthermore, peptide mimetics are generally cheaper to produce than peptides. Naturally, those skilled in the art will recognize that the design of a peptidomimetic may require slight structural alteration or adjustment of a chemical structure designed or identified using the methods of the invention. In general, chemical compounds or peptides identified or designed based on the ISVDs of the invention can be synthesized chemically and then tested for ability to bind and modulate LRRK2 activity using any of the methods described herein.

The allosteric modulators may also comprise larger polypeptides comprising from about 40 to about 500 amino acids, such as antibodies, antibody mimetics, antibody fragments or antibody conjugates as previously described herein. The LRRK2 binding agents, including protein binding agents and/or binding agent preferably having allosteric activity or allosteric modulating activity upon binding to LRRK2, defining these allosteric compounds as allosteric modulators, performing as positive allosteric modulators (PAMs) of LRRK2 when increasing its (kinase) activity, or vice versa, as negative allosteric modulators (NAMs) of LRRK2 activity, resulting in decreased LRRK2 (kinase) activity, or inhibiting or blocking LRRK2 activity. It is envisaged in a specific embodiment to provide for a LRRK2 allosteric modulator comprising an antibody or active antibody fragment as defined herein. Said allosteric modulator preferably comprising an ISVD which specifically binds LRRK2 and contains 4 framework regions and 3 CDR regions.

More specifically said ISVDs, Nanobodies or VHHs or active antibody fragments comprising a CDR1, CDR2, and CDR3 sequence as provided for the CDRS of the ISVDs of SEQ ID NO:1-19, or any combination of said CDR sequences thereof. The CDR region annotation for each Nb sequence described herein is shown in Table 3 for the specific CDR annotation used in the current analysis. Alternatively, slightly different CDR annotations known in the art may be applied here to define the CDR regions, and relate to the AbM (AbM is Oxford Molecular Ltd.'s antibody modelling package as described on http://www.bioinf.org.uk/abs/index.html), Chothia (Chothia and Lesk, 1987; J Mol Biol. 196:901-17), Kabat (Kabat et al., 1991; Sequences of Proteins of Immunological Interest. 5th edition, NIH publication 91-3242), or IMGT (LeFranc, 2014; Frontiers in Immunology. 5 (22): 1-22) annotation, which are all applicable to identify the CDR regions of the ISVDs as disclosed herein for SEQ ID NO: 1-19. To clarify the exact sequences covered by said alternative annotations, the CDRs are provided as an example relative to the currently applied annotation of Table 3, for CA12610 Nb in FIG. 10.

It should be noted that—as is well known in the art for VH domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. The total number of amino acid residues in a VH domain and a VHH domain will usually be in the range of from 110 to 120, often between 112 and 115. It should however be noted that smaller and longer sequences may also be suitable for the purposes described herein.

In a specific embodiment, said ISVDs or Nbs specifically binding LRRK2 affect kinase activity in cells (also see Examples and in particular FIGS. 2A-2D). The effect may be envisaged in a specific embodiment as a kinase inhibitory effect, by directly or indirectly acting on the LRRK2 kinase enzymatic activity, observed as an inhibition of LRRK2 autophosphorylation and/or inhibition of substrate (Rab10) phosphorylation in cellulo, and/or in vitro LRRK2 kinase activity inhibition toward a peptide substrate. So in one embodiment, the LRRK2 allosteric modulator described herein inhibits kinase activity in cells.

More specifically, a panel of the Nbs as exemplified herein (Nb1, Nb6, Nb23 and Nb42) with the functionality of inhibiting all such tested LRRK2 kinase activities, i.e. inhibition of LRRK2 autophosphorylation and inhibition of substrate (Rab10) phosphorylation in cellulo, and/or in vitro LRRK2 kinase activity inhibition toward a peptide substrate, thus block LRRK2 kinase activity per se. Nb42 was also classified in this group, as it has a strong inhibitory effect on LRRK2 autophosphorylation and Rab phosphorylation in cells, though the in vitro kinase activity effect could not be shown in the presence of Nb42. So in a specific embodiment envisaging the Nbs exemplified herein, the LRRK2 allosteric modulators which are inhibitors of LRRK2 kinase activity per se, are defined herein as comprising an ISVD wherein the CDR1, CDR2 and CDR3 consist of the CDRS of SEQ ID NO: 1, 2, 3, or 6, wherein the CDR regions are defined as in Table 3-4 or FIG. 10 disclosed herein. These kinase inhibitory Nbs provide for two categories based on their binding epitopes. While Nb1 (SEQ ID NO:1) and Nb6 (SEQ ID NO:2) bind exclusively to the C-terminal part of the COR domain (COR-B), Nb23 (SEQ ID NO:3) cross-links with K2078 and K2091 in the C-terminal lobe of the kinase domain. The latter residues are located in close proximity to each other and to the S1292 autophosphorylation site, but are located quite far from the ATP binding pocket. The observation that none of these Nbs bind in the kinase ATP-binding pocket, while Nb1 and Nb6 even bind outside the kinase domain, indicates that these Nbs do not act via an ATP-competitive mechanism. Correspondingly, kinetic analysis shows that all three Nbs act via a mixed-type inhibition mechanism, with a preference of binding to an “ATP-free” LRRK2 conformation, over an “ATP-bound” conformation. Especially considering that the COR-B domain is located centrally in the LRRK2 structure, this could indicate that these Nbs push the LRRK2 protein to a more “open”, non-catalytically competent conformation. Nevertheless, none of these Nbs induce microtubule relocalization of LRRK2 in contrast to ATP-competitive type 1 inhibitors.

A second group of Nbs (Nb17, Nb36, Nb38, Nb40, and Nb41) envisaged herein inhibits Rab substrate phosphorylation in cells, while leaving autophosphorylation and peptide phosphorylation unaffected, suggesting these Nbs either sterically interfere with binding of the larger Rab substrates or fix LRRK2 in a conformation that excludes Rab binding. So in a specific embodiment envisaging the Nbs exemplified herein, the LRRK2 allosteric modulators which are inhibitors of LRRK2 kinase activity in that they prevent substrate phosphorylation, are defined herein as comprising an ISVD wherein the CDR1, CDR2 and CDR3 consist of the CDRS of SEQ ID NO:4, 5, 7, 8, or 12, wherein the CDR regions are defined as in Table 3-4 or FIG. 10 disclosed herein.

A further embodiment relates to Nbs of the third group (Nb22, Nb28) that modulate LRRK2 through increasing the LRRK2 kinase activity both in cells (and in vitro, as shown at least for Nb22). So in a specific embodiment envisaging the Nbs exemplified herein, the LRRK2 allosteric modulators which are activating LRRK2 kinase activity, are defined herein as comprising an ISVD wherein the CDR1, CDR2 and CDR3 consist of the CDRS of SEQ ID NO: 9 or SEQ ID NO: 18, wherein the CDR regions are defined as in Table 3-4 or FIG. 10 disclosed herein.

Finally, a fourth group of Nbs can be envisaged (Nb3, Nb9, Nb10, Nb13, Nb31, Nb37 and Nb39) which does not seem to influence LRRK2 kinase activity, though still provides for the previously mentioned effects that these also bind to LRRK2 outside the ATP-binding pocket with high affinity (nanomolar range) and retain its cytoplasmic distribution. Those kinase neutral binding agents may be particularly suited for use in detection or diagnostic assays requiring LRRK2 binding, or alternatively for use in screening assays that require LRRK2 conformations accessible upon binding with these Nbs. So in a specific embodiment envisaging the Nbs exemplified herein, the LRRK2 Nbs which are kinase neutral binders with high affinity, are defined herein as comprising an ISVD wherein the CDR1, CDR2 and CDR3 consist of the CDRS of SEQ ID NO: 10, 11, and SEQ ID NOs: 13-17, wherein the CDR regions are defined as in Table 3-4 or FIG. 10 disclosed herein.

Moreover, a panel of LRRK2 allosteric modulators are provided herein which prevent LRRK2 from association to microtubules in a cell, even when an ATP-competitive LRRK2 kinase inhibitor compound is present in the same cell. Said ATP-competitive LRRK2 kinase inhibitor may also be defined herein as an orthosteric binder of LRRK2. So in a specific embodiment envisaging the Nbs exemplified herein, the LRRK2 allosteric modulators which bind with high affinity and block the relocalization of LRRK2 in cells when such relocalization to microtubules is normally (i.e. in the absence of the modulator) triggered (e.g. by the presence of a type 1 ATP-competitive kinase inhibitor), are defined herein as comprising an ISVD wherein the CDR1, CDR2 and CDR3 consist of the CDRS of SEQ ID NO: 3, 4, 5 and 9, wherein the CDR regions are defined as in Table 3-4 or FIG. 10 disclosed herein.

In another embodiment, the LRRK2 allosteric modulator comprises an ISVD comprising the amino acid sequence selected from the group consisting of SEQ ID NO: 1-19. In another embodiment, the LRRK2 allosteric modulator comprises an ISVD comprising the amino acid sequence selected from the group consisting of a sequence with at least 85% identity to any of the sequences of SEQ ID NO:1-19, wherein the CDRs are identical to the CDRs of SEQ ID NO: 1-19, and differences may be present in Framework residues. In a specific embodiment, the LRRK2 allosteric modulator comprises an ISVD comprising the amino acid sequence selected from the group consisting of a sequence with at least 85% identity to any of the sequences of SEQ ID NO: 1-19, or at least 90% identity thereof, or at least 95% identity thereof, wherein the CDRs are identical to the CDRs of SEQ ID NO: 1-19, and differences may be present in Framework residues, except for the llama germline hallmark residues present in said Framework regions. More specifically, the latter FR residues that should not be altered correspond to residues 37 (Kabat No; F or Y) which corresponds to residue 39 of SEQ ID NO:1 for instance, residue 44-45 (Kabat No; QR, ER or DR), corresponding to residues 46-47 of SEQ ID NO:1, residue 47 (Kabat No; L, T or F) corresponding to residue 49 of SEQ ID NO:1, residue 78 (Kabat No; G or V) corresponding to residue 80 from SEQ ID NO:1, and residue 84 (Kabat No; P), corresponding to residue 89 from SEQ ID NO:1 for instance (see also FIG. 10, underlined/bold residues of first sequence). In another embodiment, said LRRK2 modulator comprises and ISVD comprising the amino acid sequence selected from the group consisting of a humanized variant of any of the sequences of SEQ ID NO: 1-19, or a humanized variant of any of the sequences with at least 85% identity to SEQ ID NO: 1-19, or with at least 90% identity to SEQ ID NO:1-19, or with at least 95% identity to SEQ ID NO: 1-19, wherein the CDRs are identical to any one of the CDRs of SEQ ID NO: 1-19, and the FR hallmark residues are identical to the one of any one of SEQ ID NO: 1-19, and with humanization substitutions that provide differences in residues elsewhere in the FR regions.

The term ‘humanized variant’ of an immunoglobulin single variable domain such as a domain antibody and Nanobody® (including VHH domain) refers to an amino acid sequence of said ISVD representing the outcome of being subjected to humanization, i.e. to increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other or further suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody, may have several advantages, such as a reduced immunogenicity, compared to the corresponding naturally occurring VHH domains. In summary, the humanizing substitutions should be chosen such that the resulting humanized amino acid sequence of the ISVD and/or VHH still retains the favorable properties, such as the antigen-binding capacity, and allosteric modulation capacity. The skilled person will be able to select humanizing substitutions or suitable combinations of humanizing substitutions which optimize or achieve a desired or suitable balance between the favorable properties provided by the humanizing substitutions on the one hand and the favorable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also, a subset of human germline alleles most homologous to the target sequence may be aligned as starting point to identify suitable humanisation residues. Alternatively, the VHH is analyzed to identify its closest homologue in the human alleles and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues (i.e. hallmark residues, bold/underlined in FIG. 10) in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization. Suitable mutations, in particular substitutions, can be introduced during humanization to generate a polypeptide with reduced binding to pre-existing antibodies (reference is made for example to WO 2012/175741 and WO2015/173325), for example in at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or preferably at one or more other framework residues (i.e. non-Hallmark residues) or any suitable combination thereof. Depending on the host organism used to express the amino acid sequence, ISVD, VHH or polypeptide of the invention, such deletions and/or substitutions may also be designed in such a way that one or more sites for posttranslational modification (such as one or more glycosylation sites at asparagine to be replaced with G, A, or S; and/or Methionine oxidation sites) are removed, as will be within the ability of the person skilled in the art. Alternatively, substitutions or insertions may be designed so as to introduce one or more sites for attachment of functional groups, for example to allow site-specific pegylation. In some cases, at least one of the typical Camelidae hallmark residues with hydrophilic characteristics at position 37, 44, 45 and/or 47 is replaced (Kabat No; see WO2008/020079 Table A-03). Another example of humanization includes substitution of residues in FR 1, such as position 1, 5, 11, 14, 16, and/or 23, and/or 28; in FR2 such as positions 40 and/or 43; in FR3, such as positions 60-64, 73, 74, 75, 76, 78, 79, 81, 82b, 83, 84, 85, 93 and/or 94; and in FR4, such as position 103, 104, 105, 108 and/or 111 (see WO2008/020079 Tables A-05-A08; all numbering according to the Kabat). In one embodiment said humanized variant includes at least one substitution in any one of the ISVDs comprising SEQ ID NO:1-19 selected from the group of substitutions at the following positions (according to Kabat No): residue 1 substitution to E or D; residue 14 to P; residue 23 to A; 40 to A; 43 to K; 60 to A; 61 to D; 62 to S; 63 to V; 64 to K; 73 to A; 76 to N; 81 to Q; 83 to R; 85 to E; 103 to W; 105 to Q and/or 108 to L.

More preferably, said humanized variant includes at least one substitution in any one of the ISVDs comprising SEQ ID NO:1-19 selected from the group of substitutions at the following positions (according to Kabat No): residue 1 substitution to E or D; residue 14 to P; 73 to A; 81 to Q; 83 to R; 85 to E; 105 to Q and/or 108 to L. In another specific embodiment, the humanization substitutions of at least SEQ ID NO:1 and/or SEQ ID NO:2 as described herein results in at least substitution of a few residues in FR3, in particular substitution of positions 60-62 and/or 60-64 (in particular for SEQ ID NO:2).

In another embodiment, the LRRK2 allosteric modulator comprises an ISVD comprising the amino acid sequence selected from the group consisting of SEQ ID NO:1-10. In another embodiment, the LRRK2 allosteric modulator comprises an ISVD comprising the amino acid sequence selected from the group consisting of a sequence with at least 85% identity to any of the sequences of SEQ ID NO:1-10, wherein the CDRs are identical to the CDRs of SEQ ID NO:1-10, and differences may be present in Framework residues. In a specific embodiment, the LRRK2 allosteric modulator comprises an ISVD comprising the amino acid sequence selected from the group consisting of a sequence with at least 85% identity to any of the sequences of SEQ ID NO:1-10, wherein the CDRs are identical to the CDRs of SEQ ID NO:1-10, and differences may be present in Framework residues, except for the llama germline hallmark residues present in said Framework regions. In another embodiment, said LRRK2 modulator comprises and ISVD comprising the amino acid sequence selected from the group consisting of a humanized variant of any of the sequences of SEQ ID NO:1-10, or a humanized variant of any of the sequences with 85% identity to SEQ ID NO:1-10, wherein the CDRs are identical to the CDRs of SEQ ID NO:1-10 and differences may be present in the FR regions.

Another embodiment relates to a LRRK2 allosteric modulator as a multi-specific agent, comprising at least one allosteric LRRK2 modulator as described herein, resulting in a multiparatopic LRRK2 modulator, binding to several different binding sites of LRRK2, or in a multivalent LRRK2 modulator, which may increase the avidity for binding to LRRK2, or another form of a multi-specific LRRK2 allosteric modulator, including a binding agent with a different target specificity. A “multi-specific” form of a LRRK2 allosteric ISVD for instance, is formed by bonding together two or more immunoglobulin single variable domains, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) protein binding agent of the invention may be suitably directed against two or more different epitopes on the same antigen, for example against epitope 1 on one domain and epitope 2 on another domain of LRRK2; or may be directed against two or more different antigens, for example against LRRK2 and one as a half-life extension against Serum Albumin. One of the most widely used techniques for increasing the half-life and/or reducing immunogenicity of pharmaceutical proteins comprises attachment of a suitable pharmacologically acceptable polymer, such as poly-ethyleneglycol (PEG) or derivatives thereof (such as methoxypoly-ethyleneglycol (mPEG)). Another technique for increasing the half-life of a binding domain may comprise the engineering into bifunctional or bispecific domains (for example, one or more ISVDs or active antibody fragments against LRRK2 coupled to one ISVD or active antibody fragment against serum albumin aiding in prolonging half-life)) or into fusions of antibody fragments, in particular immunoglobulin single variable domains, with peptides (for example, a peptide against a serum protein such as albumin). The coupling to additional moieties will result in multispecific binding agent, as further disclosed herein.

Multivalent or multi-specific binding agents of the invention may also have (or be engineered and/or selected for) increased avidity and/or improved selectivity for the desired LRRK2 interaction, and/or for any other desired property or combination of desired properties that may be obtained by the use of such multivalent or multi-specific binding agents. For instance, the combination of one or more ISVDs binding epitope 1, and one or more ISVDs binding epitope 2 as described herein, results in a multi-specific binding agent of the invention with higher modulating activity. Said multi-specific binding agent comprises at least said binding agents directed against epitope 1 and epitope 2, which may be coupled via a linker, spacer. Upon binding LRRK2, said multi-specific binding agent or multivalent ISVD may have an additive or synergistic impact on the LRRK2 allosteric modulating activity. The multispecific LRRK2 allosteric modulator of the invention may be coupled to a functional moiety, a targeting moiety, a half-life extending moiety, or to a cell penetrant carrier.

Given that modulation of LRRK2 activity is desired in treating a number of neurological disorders, the multispecific LRRK2 allosteric modulator may as a non-limiting example comprise a functional moiety able to cross the blood-brain-barrier, or may be further fused or chemically coupled to a moiety that is able to cross the blood-brain-barrier for example by receptor mediated transcytosis. Indeed, the blood-brain interfaces severely restrict the cerebral bioavailability of pharmaceutical agents and compounds. Because of the limited penetration of for example antibodies, active antibody fragments or small molecules, high amounts need to be administered to obtain the desired efficacy. Besides the risk of high dosing on inducing peripheral side effects in the patient, such an approach is also economically undesirable, in view of societal costs, as well as the need for mass-production capacities, especially in larger indications such as Parkinson's disease and Alzheimer's disease with millions of patients, where the manufacturing of biologicals may be an important limiting factor. A number of means and methods to efficiently shuttle compounds over the BBB have been reported (e.g. WO2015031673A2; WO2014033074A1; WO2015124540A1; WO2015191934A2), though the type of the BBB-crossing functional moiety is still to be selected on a case-by-case and trial and error approach. The present invention thus provides for a multispecific LRRK2 allosteric modulator that may comprise for instance a (single domain) antibody that targets a blood brain barrier (BBB) receptor. This multispecific LRRK2 allosteric modulator can be injected intravenous after which the BBB receptor targeting antibody (or single variable domain antibody) will shuttle the complex across the BBB. Though further delivery methods and vehicles that may be suitable comprise delivery via nanoparticles, or lipid-based delivery systems such as artificial exosomes, which may also be cell-specific, and suitable for delivery of the binding agents or multi-specific binding agents as a protein or in the form of DNA (nucleic acid, vector) to encode said binding agent or modulator [48-49].

Indeed, another aspect of the invention relates to a nucleic acid molecule comprising a nucleic acid sequence encoding the LRRK2 binding agent or allosteric modulator as described herein. “Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, the (reverse) complement DNA, and RNA. It also includes known types of modifications, for example, methylation, “caps” substitution of one or more of the naturally occurring nucleotides with an analog. By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes comprising non-native nucleic acid sequences, and the like. “Coding sequence” is a nucleotide sequence, which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

One embodiment discloses an expression cassette comprising said nucleic acid molecule. More specific embodiments disclose the expression cassette wherein elements for cell- or tissue-specific expression are present. An “expression cassette” comprises any nucleic acid construct capable of directing the expression of a gene/coding sequence of interest, which is operably linked to a promoter of the expression cassette. Expression cassettes are generally DNA constructs preferably including (5′ to 3′ in the direction of transcription): a promoter region, a polynucleotide sequence, homologue, variant or fragment thereof operably linked with the transcription initiation region, and a termination sequence including a stop signal for RNA polymerase and a polyadenylation signal. It is understood that all of these regions should be capable of operating in biological cells, such as prokaryotic or eukaryotic cells, to be transformed. The promoter region comprising the transcription initiation region, which preferably includes the RNA polymerase binding site, and the polyadenylation signal may be native to the biological cell to be transformed or may be derived from an alternative source, where the region is functional in the biological cell. Such cassettes can be constructed into a “vector”. Further embodiments relate to a vector comprising said expression cassette or said nucleic acid molecule of which the sequence encodes the LRRK2 allosteric modulator as described herein.

The term “vector”, “vector construct,” “expression vector,” or “gene transfer vector,” as used herein, is intended to refer to a nucleic acid molecule capable of transporting another nucleic acid molecule to which it has been linked. More particular, said vector may include any vector known to the skilled person, including any suitable type, but not limited to, for instance, plasmid vectors, cosmid vectors, phage vectors, such as lambda phage, viral vectors, even more particular a lentiviral, adenoviral, AAV or baculoviral vectors, or artificial chromosome vectors such as bacterial artificial chromosomes (BAC), yeast artificial chromosomes (YAC), or P1 artificial chromosomes (PAC). Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments. The construction of expression vectors for use in transfecting cells is also well known in the art, and thus can be accomplished via standard techniques (see, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.).

Furthermore, an alternative embodiment relates to the use of said nucleic acid molecule, expression cassette, or vector described herein encoding said LRRK2 allosteric modulator, for production as an intrabody. An intracellular antibody or “intrabody” is an antibody or an active fragment of an antibody that is heterologously expressed within a designated intracellular compartment, a process which is made possible through the in-frame incorporation of intracellular trafficking signals. Intrabodies exert their functions upon exquisitely specific interaction with target antigens. This results in interruption or modification of the biological functions of the target protein. An intrabody can be expressed in any shape or form such as an intact IgG molecule or a Fab fragment. More frequently, intrabodies are used in genetically engineered antibody fragment format and structures of scFv intrabodies, single domain intrabodies, or bispecific tetravalent intradiabodies. For a review see Zhu, and Marasco, 2008 (Therapeutic Antibodies. Handbook of Experimental Pharmacology 181. _c Springer-Verlag Berlin Heidelberg). The LRRK2 allosteric modulator as described herein, possibly encoded by a nucleic acid molecule or expression cassette or present on a vector as described herein, resulting in an intrabody upon expression within a suitable host system, could also serve as a tool to further investigate LRRK2 signaling, as a diagnostic, for in vivo imaging, or as well as a therapeutic, when an applicable form of gene delivery is identified. A skilled person is aware about the currently applied methodologies of administration and delivery (also see Zhu and Marasco 2008).

The field of gene therapy for the nervous system has undergone explosive growth in the last 5 years, with human clinical trials for gene replacement in progress and with the recent approval of a gene therapy for spinal muscle atrophy [86]. Systems based on adeno-associated virus (AAV) are increasingly used in clinical trials as they efficiently transduce both dividing and non-dividing cells, provide long-term transgene expression (after a single administration) and have low intrinsic toxicity [87], and various gene therapy strategies have been developed for PD [88]. Where said (multispecific) LRRK2 allosteric modulator is provided as a nucleic acid or a vector, it is particularly envisaged that the modulator is administered through gene therapy. ‘Gene therapy’ as used herein refers to therapy performed by the administration to a subject of an expressed or expressible nucleic acid. For such applications, the nucleic acid molecule or vector as described herein allow for production of the LRRK2 allosteric modulator within a cell. A large number of methods for gene therapy are available in the art and include, for instance (adeno-associated) virus mediated gene silencing, or virus mediated gene therapy (e.g. US 20040023390; Mendell et al 2017, N Eng J Med 377:1713-1722). A plethora of delivery methods are well known to those of skill in the art and include but are not limited to viral delivery systems, microinjection of DNA plasmids, biolistics of naked nucleic acids, use of a liposome. In vivo delivery by administration to an individual patient occurs typically by systemic administration (e.g., intravenous, intraperitoneal infusion or brain injection; e.g. Mendell et al 2017, N Eng J Med 377:1713-1722). Where said (multispecific) LRRK2 allosteric modulator is provided as a nucleic acid or a vector, it is more particularly also envisaged that the modulator is administered through delivery methods and vehicles that comprise nanoparticles or lipid-based delivery systems such as artificial exosomes, which may also be cell-specific, and suitable for delivery of the binding agents or multi-specific binding agents as intrabodies or in the form of DNA to encode said binding agent or modulator [48-49].

In another embodiment, the LRRK2 binding agent, ISVD, and/or allosteric modulator as purified protein, as nucleic acid, or expression cassette or vector as described herein may also be included in a kit, for instance to apply as a tool in LRRK2 signaling studies, or for LRRK2 structural biology and biochemistry analysis.

A further aspect relates to said LRRK2-specific ISVDs, the nucleic acid molecule or the vector encoding said LRRK2-specific binding agents, or the pharmaceutical composition comprising these, as described herein, for use as a diagnostic.

In a particular embodiment, kits are provided which contain means to detect LRRK2 protein, including the LRRK2 allosteric modulator or binding agent or ISVDs as described herein, allowing to detect or modulate LRRK2 signaling in a system, which may be an in vitro or in vivo system. It is envisaged that these kits are provided for a particular purpose, such as for modulating LRRK2, or for in vivo imaging, or for diagnosis of an altered LRRK2 quantity, response or effect in a subject. In another embodiment, said kit is provided which contains means including a nucleic acid molecule, a vector, or a composition as described herein. The means further provided by the kit will depend on the methodology used in the application, and on the purpose of the kit. For instance, detection of a labelled LRRK2 allosteric modulator or binding agent, ISVD, or nucleic acid molecule as described herein, which may be desired for LRRK2 quantification on nucleic acid or protein level. For protein-based detection, the kits typically will contain labelled or coupled LRRK2 binding agents such as ISVDs. Likewise, for detection at the nucleic acid level, the kits may contain labels for nucleic acids such as primers or probes. Further control agents, antibodies or nucleic acids may also be provided in the kit. A standard, for reference or comparison, a LRRK2 substrate or signaling component, a reporter gene or protein or other means for using the kit may also be included. Of course, the kit may further comprise pharmaceutically acceptable excipients, buffers, vehicles or delivery means, an instruction manual and so on.

Another aspect of the invention provides for a method for detecting the presence, absence or level of LRRK2 protein in a sample, the method comprising: contacting the sample with the LRRK2 binding agent or ISVD as described herein, and detecting the presence or absence or level, i.e. quantifying, the bound LRRK2 ISVD, which is optionally a labelled, conjugated or multispecific LRRK2 binding agent. The sample used herein may be a sample isolated from the body, such as a body fluid, including blood, serum, cerebrospinal fluid, among others, or may be an extract, such as a protein extract, a cell lysate, etc.

Furthermore, the LRRK2 allosteric modulator or binding agent, in particular comprising a LRRK2-specific ISVD, the nucleic acid molecule, the vector, or the pharmaceutical composition comprising said LRRK2-specific binding agent, as described herein may also be used for in vivo imaging.

For the purpose of detection and/or imaging, in vitro or in vivo, the LRRK2 binding agent, comprising a LRRK2-specific ISVD, as described herein may further comprise in some embodiments a detection agent, such as a tag or a label. For instance, the ISVDs, VHHs, or Nbs as exemplified herein were also tagged, by the 6-His-EPEA double tag (as presented in SEQ ID NO:20; for EPEA tag: see also WO2011/147890A1). Such a tag allows affinity purification and detection of the antibody or active antibody fragments of the invention.

Some embodiments comprise the LRRK2 binding agent, ISVD, or allosteric modulator, further comprising a label or tag, or more specifically, the LRRK2 binding agent labelled with a detectable marker. The term detectable label or tag, as used herein, refers to detectable labels or tags allowing the detection and/or quantification of the LRRK2 modulator or binding agent as described herein, and is meant to include any labels/tags known in the art for these purposes. Particularly preferred, but not limiting, are affinity tags, such as chitin binding protein (CBP), maltose binding protein (MBP), glutathione-S-transferase (GST), poly(His) (e.g., 6× His or His6), biotin or streptavidin, such as Strep-tag®, Strep-tag II® and Twin-Strep-tag®; solubilizing tags, such as thioredoxin (TRX), poly(NANP) and SUMO; chromatography tags, such as a FLAG-tag; epitope tags, such as V5-tag, myc-tag and HA-tag; fluorescent labels or tags (i.e., fluorochromes/-phores), such as fluorescent proteins (e.g., GFP, YFP, RFP etc.) and fluorescent dyes (e.g., FITC, TRITC, coumarin and cyanine); luminescent labels or tags, such as luciferase, bioluminescent or chemiluminescent compounds (such as luminal, isoluminol, theromatic acridinium ester, imidazole, acridinium salts, oxalate ester, dioxetane or GFP and its analogs); phosphorescent labels; a metal chelator; and (other) enzymatic labels (e.g., peroxidase, alkaline phosphatase, beta-galactosidase, urease or glucose oxidase); radioisotopes. Also included are combinations of any of the foregoing labels or tags. Technologies for generating labelled polypeptides and proteins are well known in the art. A LRRK2 allosteric modulator or binding agent comprising a LRRK2-specific ISVD of the invention, coupled to, or further comprising a label or tag allows for instance immune-based detection of said bound LRRK2-specific agent. Immune-based detection is well known in the art and can be achieved through the application of numerous approaches. These methods are generally based upon the detection of a label or marker, such as described above. See, for example, U.S. Pat. Nos. 3,817,837; 3,850,752; 3,939,350; 3,996,345; 4,277,437; 4,275,149 and 4,366,241. In the case where multiple antibodies are reacted with a single array, each antibody can be labelled with a distinct label or tag for simultaneous detection. Yet another embodiment may comprise the introduction of one or more detectable labels or other signal-generating groups or moieties, or tags, depending on the intended use of the labelled or tagged LRRK2 allosteric modulator or binding agent of the present invention. Other suitable labels will be clear to the skilled person, and for example include moieties that can be detected using NMR or ESR spectroscopy. Such labelled LRRK2 allosteric modulators, such as LRRK2-specific ISVDs or Nanobodies as described herein may for example be used for in vitro, in vivo or in situ assays (including immunoassays known per se such as ELISA, RIA, EIA and other “sandwich assays”, etc.) as well as in vivo imaging purposes, depending on the choice of the specific label.

So, in another aspect, an in vitro method is disclosed for detection of the localization and distribution of human LRRK2 protein in a biological sample, comprising the steps of: reacting the sample with a LRRK2 binding agent, comprising a LRRK-specific ISVD as described herein, and detecting, the localization and distribution of said LRRK2 binding in said biological sample. The biological sample as used herein may envisage any sample derived from a biological system, and for example comprise cells of brain tissue, or an extract or an in vitro sample, or a body fluid such as cerebrospinal fluid or blood.

Another aspect of the invention relates to a pharmaceutical composition comprising one or more LRRK2 allosteric modulators as described herein, or comprising the nucleic acid molecule, or vector as described herein, and optionally a pharmaceutically acceptable carrier or diluent. These pharmaceutical compositions can be utilized to achieve the desired pharmacological effect by administration to a patient in need thereof. A “pharmaceutically or therapeutically effective amount” of compound or binding agent or composition is preferably that amount which produces a result or exerts an influence on the particular condition being treated. The LRRK2 allosteric modulator or the pharmaceutical composition as described herein may also function as a “therapeutically active agent” which is used to refer to any molecule that has or may have a therapeutic effect (i.e. curative or stabilizing effect) in the context of treatment of a disease (as described further herein). Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)—Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles. Such pharmaceutical composition comprising said LRRK2 allosteric modulator may also concern a nanoparticle containing composition or lipid-based exosome delivery vehicle, as discussed herein [48-49].

The allosteric modulator or binding agent or the pharmaceutical composition as described herein may act as a therapeutically active agent, when beneficial in treating LRRK2-related diseases. The pharmaceutical composition as described herein may also comprise a multi-specific LRRK2 allosteric modulator which may contain or be coupled to additional functional groups or moieties, advantageous when administrated to a subject.

Moreover, the pharmaceutical composition as described herein comprising the LRRK2 allosteric modulator, may further comprise a compound known as a LRRK2 kinase activity inhibitor, preferably a Type I kinase inhibitor. Said known LRRK2 kinase inhibitor of Type I inhibition of a kinase is also known to induce LRRK2 relocalization to the microtubules, as described above [36].

In contrast to these state-of-the-art Type I ATP-competitive kinase inhibitors, the LRRK2 allosteric modulators described herein, such as the ISVDs disclosed herein, do not induce a microtubular redistribution of LRRK2 protein (e.g. see Example 6). Furthermore, an increased microtubule binding or relocalization of LRRK2 protein is not only observed when ATP-competitive kinase inhibitors are binding LRRK2 in a cell, but also upon overexpression of a plethora of pathological LRRK2 mutants. Recent reports have associated such LRRK2 relocalization events with potentially blocked transport along microtubules [36, 40]. The LRRK2 allosteric modulators comprising LRRK2-specific ISVDs of the present invention are different in the sense that they do not engage in relocalization of LRRK2, which further establishes that the LRRK2 allosteric modulators or ISVDs disclosed herein provide for a conformational LRRK2 binding within the cell which is different as compared to ‘classical’ ATP-competitive or known Type I kinase inhibitors. Moreover, a panel of the LRRK2 allosteric modulators described herein functions in decreasing or rescuing the microtubular localisation when added to cells comprising LRRK2 protein relocated at the microtubules (e.g. due to prior treatment with a small compound binding and inhibiting LRRK2 kinase activity as a type I inhibitor, or due to PD mutant LRRK2). The surprising finding that said subset of ISVDs can even revert microtubular redistribution of LRRK2 upon inhibition with ATP competitive kinase inhibitors provides for further opportunities to apply these LRRK2 allosteric modulators as (combination) therapeutics as this could be beneficial to avoid any undesired effects upon treatment with LRRK2 inhibitors.

Another embodiment relates to said LRRK2 allosteric modulator as described herein, the nucleic acid molecule, the vector or the pharmaceutical composition as described herein for use as a medicament. More particularly, for use in treatment of a LRRK2-related or -associated disorder or disease. “LRRK2-related disorders” as used herein comprises the diseases currently known to be associated with and impacted by or cause through changes in LRRK2 activity, most prominently include Parkinson's disease (Zimprich A, et al. (2004) Neuron 44:601-607; Paisan-Ruiz C, et al. (2004) Neuron 44:595-600), as well as Crohn's disease (Ridler C. (2018) Nat Rev Neurol. 14(3):126; Hui K Y et al (2018) Sci Transl Med. 10(423); Rivas M A et al (2018) PLoS Genet. 14(5):e1007329); and may further be associated to immune responses as ‘host response to pathogens’ (Gardet A et al (2010) J Immunol. 185(9):5577-85); to increased risk of cancer (Saunders-Pullman et al (2010) Mov Disord. 25(15): 2536-2541; Bjorg Johanne Waro & Jan O. Aasly (2018) Brain Behav. 8(1): e00858), as well as Alzheimer's disease (Zhao Y (2011) Neurobiol Aging. 2011 November; 32(11):1990-3).

A specific embodiment relates to said LRRK2 allosteric modulator as described herein, the nucleic acid molecule, the vector or the pharmaceutical composition as described herein for use in treatment of Parkinson's disease.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Generation of LRRK2-Specific Nanobodies

LRRK2 is a large and complex protein containing several domains: an armadillo domain, an ankyrin repeat domain, a leucine-rich repeat (LRR) domain, a RocCOR (Ras of complex proteins/C-terminal of Roc) supra-domain, a kinase domain and a WD40 domain (FIG. 1B) [13-15]. As such the protein bears a rather unique combination of two catalytic activities: GTPase activity mediated by the Roc domain and Ser/Thr protein kinase activity [16]. Although, the mechanism of LRRK2 is still largely unknown, it is expected that it is regulated in a complex way and undergoes large conformational changes during its functional cycle. One source of conformational changes within LRRK2 is mediated by nucleotide (GDP vs. GTP) binding to its RocCOR domains. Indeed, our previous studies on bacterial LRRK2 homologues has shown that GTP binding leads to monomerization of the LRRK2 dimer, associated with secondary domain motions within each subunit [17,18]. Moreover, recent in vitro and in cellulo data have shown that also human LRRK2 can cycle between a monomeric and dimeric form, but the exact nature of these conformational changes remains elusive [19-22]. Here, we set out to identify Nbs that can modulate the LRRK2 activity by specifically binding to LRRK2 in one of its conformational states. In total, three immunizations using different llamas were performed using different protein constructs or conformational states of LRRK2 (FIG. 7). In a first immunization strategy (immunization 1) we immunized a llama with the LRRK2 RocCOR construct, and after immunization Nbs were selected using a phage display panning approach using full-length LRRK2 as the bait protein. Subsequently, to increase the chances for obtaining Nbs that bind LRRK2 in a specific nucleotide-induced conformation, we performed two additional immunizations, using either LRRK2 bound to and in presence of a large excess of GTPγS (a non-hydrolysable GTP analogue) (immunization 2) or LRRK2 bound to and in presence of a large excess of GDP (immunization 3). Moreover, in order to “trap” the protein in its nucleotide-specific conformation upon immunization, we performed a mild crosslinking using the lysine specific crosslinker DSS (FIGS. 8A and 8B). After immunization, Nbs were selected using phage display panning using non-crosslinked full-length LRRK2 in presence of an excess of either GTPγS (for selections from the library originating from immunization 2) or GDP (for selections from the library originating from immunization 3). Additionally, to enrich for Nbs binding to the Roc domain, the Nb libraries were subjected to two or three rounds of phage display using either GTPγS- or GDP-bound Roc protein.

These different strategies finally resulted in libraries of the selected Nb ORFs cloned in the pMESy4 vector. These vectors were sent for sequencing and based upon the CDR3 sequence the Nbs were classified in different sequence families (each Nb family displays a unique CDR3 sequence). This resulted in 49 Nb families originating from immunization 1, 70 Nb families originating from immunization 2 with selection against LRRK2-GTPγS, 4 Nb families originating from immunization 2 with selection against Roc-GTPγS, 44 Nb families originating from immunization 3 with selection against LRRK2-GDP, and 1 Nb family originating from immunization 3 with selection against Roc-GDP.

Further selection of Nbs was based on an ELISA screening step. A large subset of the sequenced Nbs were expressed in E. coli in small scale in a 96-deep-well plate format. After cell lysis the crude cell lysates were used to test binding in ELISA to full-length LRRK2 coated on the bottom of the ELISA plate. Finally, 42 Nbs from different families and resulting from the different immunization and selection strategies were selected based on a good signal in ELISA (Table 1). These Nbs were expressed in larger scale in E. coli and purified to homogeneity (FIG. 9).

TABLE 1
List of purified LRRK2-specific Nbs.
The selection of Nbs resulting from
(1) the immunization 1 with RocCOR
(RocCOR-GppNHp) and selection with full-length LRRK2, (2) the
immunization 2 with cross-linked LRRK2-GTPγS (LRRK2-GTPγS
(XL)) and selection with either LRRK2-GTPγS or Roc-GTPγS, and (3)
the immunization 3 with cross-linked LRRK2-GDP (LRRK2-
GDP (XL)) and selection with either LRRK2-GDP or Roc-GDP.
The domain specificity as determined from ELISA or
cross-linking MS (only for a subset of Nbs) is also indicated.

| | | | | | Tar- |
|---|---|---|---|---|---|
| | | | | | get |
| | Data- | | | Target | Do- |
| Nb | base | | | Domain | main |
| N° | N°. | Immunization | Selection | ELISA | CL-MS |
| 1 | CA12610 | RocCOR-GppNHp | LRRK2-GDP | COR-B | Mul- |
| | | | | | tiple |
| | | | | | dom. |
| 2 | CA12612 | RocCOR-GppNHp | LRRK2-GDP | COR-B | |
| 3 | CA12614 | RocCOR-GppNHp | LRRK2-GDP | COR-B | |
| 4 | CA12616 | RocCOR-GppNHp | LRRK2-GDP | COR-B | |
| 5 | CA12617 | RocCOR-GppNHp | LRRK2-GDP | COR-B | |
| 6 | CA12618 | RocCOR-GppNHp | LRRK2-GDP | COR-B | |
| 7 | CA12620 | RocCOR-GppNHp | LRRK2-GDP | COR-B | |
| 8 | CA13597 | LRRK2-GTPγS (XL) | LRRK2-GTPγS | K-WD40 | |
| 9 | CA13598 | LRRK2-GTPγS (XL) | LRRK2-GTPγS | K-WD40 | |
| 10 | CA13599 | LRRK2-GTPγS (XL) | LRRK2-GTPγS | K-WD40 | |
| 11 | CA13600 | LRRK2-GTPγS (XL) | LRRK2-GTPγS | K-WD40 | |
| 12 | CA13601 | LRRK2-GTPγS (XL) | LRRK2-GTPγS | K-WD40 | |
| 13 | CA13602 | LRRK2-GTPγS (XL) | LRRK2-GTPγS | K-WD40 | |
| 14 | CA13603 | LRRK2-GTPγS (XL) | LRRK2-GTPγS | K-WD40 | |
| 15 | CA13604 | LRRK2-GTPγS (XL) | LRRK2-GTPγS | K-WD40 | |
| 16 | CA13605 | LRRK2-GTPγS (XL) | LRRK2-GTPγS | K-WD40 | |
| 17 | CA13606 | LRRK2-GTPγS (XL) | LRRK2-GTPγS | FL | LRR + |
| | | | | LRRK2 | Kin |
| 18 | CA13607 | LRRK2-GTPγS (XL) | LRRK2-GTPγS | FL | |
| | | | | LRRK2 | |
| 19 | CA13608 | LRRK2-GTPγS (XL) | LRRK2-GTPγS | K-WD40 | |
| 20 | CA13609 | LRRK2-GTPγS (XL) | LRRK2-GTPγS | K-WD40 | |
| 21 | CA13610 | LRRK2-GTPγS (XL) | LRRK2-GTPγS | K-WD40 | |
| 22 | CA13611 | LRRK2-GTPγS (XL) | LRRK2-GTPγS | K-WD40 | WD40 |
| 23 | CA13612 | LRRK2-GTPγS (XL) | LRRK2-GTPγS | K-WD40 | Kin |
| 24 | CA13613 | LRRK2-GTPγS (XL) | LRRK2-GTPγS | FL | |
| | | | | LRRK2 | |
| 25 | CA13614 | LRRK2-GTPγS (XL) | LRRK2-GTPγS | No | |
| | | | | binding | |
| 26 | CA13615 | LRRK2-GTPγS (XL) | LRRK2-GTPγS | FL | |
| | | | | LRRK2 | |
| 27 | CA13616 | LRRK2-GTPγS (XL) | LRRK2-GTPγS | K-WD40 | |
| 28 | CA13617 | LRRK2-GTPγS (XL) | LRRK2-GTPγS | K-WD40 | |
| 29 | CA13618 | LRRK2-GTPγS (XL) | LRRK2-GTPγS | No | |
| | | | | binding | |
| 30 | CA13619 | LRRK2-GTPγS (XL) | LRRK2-GTPγS | FL | |
| | | | | LRRK2 | |
| 31 | CA13620 | LRRK2-GTPγS (XL) | LRRK2-GTPγS | FL | |
| | | | | LRRK2 | |
| 32 | CA16069 | LRRK2-GTPγS (XL) | Roc-GTPγS | Roc | |
| 33 | CA16070 | LRRK2-GTPγS (XL) | Roc-GTPγS | Roc | |
| 34 | CA16071 | LRRK2-GTPγS (XL) | Roc-GTPγS | Roc | |
| 35 | CA16072 | LRRK2-GTPγS (XL) | Roc-GTPγS | Roc | |
| 36 | CA14130 | LRRK2-GDP (XL) | LRRK2-GDP | FL | LRR + |
| | | | | LRRK2 | Kin + |
| | | | | | WD40 |
| 37 | CA14131 | LRRK2-GDP (XL) | LRRK2-GDP | FL | |
| | | | | LRRK2 | |
| 38 | CA14133 | LRRK2-GDP (XL) | LRRK2-GDP | FL | LRR + |
| | | | | LRRK2 | Kin |
| 39 | CA14134 | LRRK2-GDP (XL) | LRRK2-GDP | FL | LRR + |
| | | | | LRRK2 | COR-B |
| 40 | CA14135 | LRRK2-GDP (XL) | LRRK2-GDP | K-WD40 | COR- |
| | | | | | B + |
| | | | | | WD40 |
| 41 | CA14136 | LRRK2-GDP (XL) | LRRK2-GDP | K-WD40 | |
| 42 | CA14259 | LRRK2-GDP (XL) | Roc-GDP | Roc | Roc |

### Example 2. Determination of the Domain-Specificity of the LRRK2-Directed Nbs

We recombinantly expressed and purified the RocCOR, Roc, COR-B and kinase-WD40 (K-WD40) domain constructs of LRRK2 (FIG. 7). Subsequently, we performed an ELISA in which all these domain constructs were coated in parallel next to each other in the same ELISA plate, and where we subsequently detected binding of the 42 purified Nbs. FIG. 1 shows the results of this ELISA. As expected, most Nbs show binding to LRRK2 and/or at least one of the domain constructs used. However, two Nbs (CA13614 and CA13618) did not show any binding and will not be regarded any further (see also Table 1). All purified Nbs (7 Nbs) that resulted from the immunization with the RocCOR domain construct (Immunization 1) turn out to specifically bind to the C-terminal subdomain of the COR domain (COR-B). Among the Nbs obtained from the immunizations and selections with full-length LRRK2 the majority bind within the K-WD40 part of the protein (18 Nbs). Another subset of Nbs (10 Nbs) resulting from these immunizations show robust binding to LRRK2 while no binding is observed to any of the individual LRRK2 domain constructs. We therefore assume that these Nbs either bind to the N-terminal region of LRRK2 (armadillo-ankyrin-LRR domains) that was not covered by individual domains in the ELISA, or that these Nbs have an epitope on the interface of two or more domains. We therefore classify the Nbs as “full-length LRRK2 binders”. To enrich in Nbs directed against the Roc domain we specifically included a selection step with either Roc-GTPγS or Roc-GDP. This resulted in 5 Nbs directed against the Roc domain (Nb32 (CA16069), Nb33 (CA16070), Nb34 (CA16071), Nb35 (CA16072), Nb42 (CA14259)). Four out of these five also gave a strong signal with the Roc domain in ELISA. For the fifth Nb (Nb42, CA14259) binding to Roc was confirmed using analytical size exclusion chromatography (data not shown). The results of the epitope mapping are summarized in FIG. 1B.

### Example 3. The LRRK2 Kinase Activity in Cells is Modulated by Nanobodies Targeting Different LRRK2 Domains

Subsequently, we wanted to test whether any of the LRRK2-directed Nbs have the capacity to bind to LRRK2 and to modulate the LRRK2 (kinase) activity in human HEK293T cells over-expressing LRRK2. Therefore, we selected a subset of 18 of the 41 purified Nbs, taking care to cover Nbs originating from the three different immunization strategies and targeting different LRRK2 domains (i.e. “full-length LRRK2”, Roc, COR-B and kinase-WD40). We also included a Nb generated in-house against a completely unrelated bacterial protein as a negative control (“irrelevant Nb”). In order to express these Nbs in human LRRK2(wild-type)-overexpressing HEK293 cells, the Nb open reading frames were first recloned to a pEGFP vector resulting in expression of a Nb fused at its C-terminus to an enhanced GFP molecule (a so called “fluobody”) [23,24]. To test binding to LRRK2 in cells, a pull-down experiment was performed using magnetic GFP-nanotrap beads. This experiment showed that all tested Nbs were able to pull-down LRRK2 under these conditions. This thus indicates that these 18 Nbs are functional as intrabodies in the context of the cytoplasm of human cells and have sufficiently high affinity to pull-down their target protein (FIG. 12).

To assess the influence of the Nbs on LRRK2 kinase activity we monitored two physiologically and disease relevant activities of LRRK2: phosphorylation of the endogenous substrate Rab10 at position T72 and LRRK2 autophosphorylation at position S1292 [25-30]. Both activities have previously been shown to be increased in the most relevant LRRK2 PD-mutants, including the common G2019S mutant. In our cellular assay we thus co-expressed LRRK2(G2019S), Rab29 and the different Nb-GFP fusions in HEK 293T cells (FIG. 2a-c). The Rab29 co-expression has previously been shown to boost LRRK2 autophosphorylation as well as Rab10 phosphorylation [31]. Among the 18 selected Nbs we have detected a group which had no influence on LRRK2 kinase activity compared to the controls (i.e. Nb3 (CA12614), Nb9 (CA13598), Nb10 (CA13599), Nb13 (CA13602), Nb31 (CA13620), Nb37 (CA14131), and Nb39 (CA14134) (FIG. 2d), while others strongly decreased LRRK2 autophosphorylation and/or Rab10 phosphorylation (Nb1 (CA12610), Nb6 (CA12618), Nb23 (CA13612), Nb42 (CA14259), Nb17 (CA13606), Nb36 (CA14130), Nb38 (CA14133), Nb40 (CA14135) and Nb41 (CA14136))(FIG. 2). In addition, several Nbs lead to a significant increase of LRRK2 kinase activity, which is most prominent for Nb28 (CA13617) and Nb22 (CA13611). Interestingly, the Nbs seem to influence LRRK2 autophosphorylation and Rab10 phosphorylation differently. While some of the binders inhibit both Rab10 phosphorylation and LRRK2 autophosphorylation at S1292 (e.g. the COR-B binders Nb1 (CA12610) and Nb6 (CA12618), the Roc binder Nb42 (CA14259), or the kinase-WD40 binders Nb23 (CA13612)), others seem to inhibit Rab10 phosphorylation while inhibiting S1292 phosphorylation at a lesser extent (e.g. the “full-length LRRK2” binders Nb17 (CA13606), Nb36 (CA14130), Nb38 (CA14133), or the “K-WD40” binders Nb40 (CA14135) and Nb41 (CA14136)). In contrast, other binders show a somewhat stronger inhibitory effect towards LRRK2 autophosphorylation (e.g. the “full-length LRRK2” binder Nb37 (CA14131) or the COR-B binder Nb3 (CA12614)).

### Example 4. Epitope Mapping by Crosslinking Mass Spectrometry (CL-MS) Reveals the Binding Epitopes of the Activity-Modulating Nbs

Following up on the above results, we decided to select ten Nbs that modulate LRRK2 (G2019S) activity in cells for a more thorough in vitro characterization. The following Nbs were chosen for further characterization: Nb17 (CA13606), Nb36 (CA14130), Nb38 (CA14133) (directed against “full-length LRRK2”, inhibiting Rab10 phosphorylation in cells), Nb39 (CA14134) (directed against “full-length LRRK2”), Nb42 (CA14259) (directed against the Roc domain, inhibiting Rab10 and autophosphorylation in cells), Nb1 (CA12610), Nb6 (CA12618) (directed against the COR-B domain, inhibiting Rab10 phosphorylation and autophosphorylation in cells), Nb22 (CA13611) (directed against the kinase-WD40 domain, activating Rab10 phosphorylation in cells), Nb40 (CA14135) and Nb23 (CA13612) (directed against the kinase-WD40 domain, inhibiting Rab10 phosphorylation and Rab10 and autophosphorylation in cells, respectively) (FIG. 2d).

First, we used a crosslinking MS approach to obtain a more detailed insight in the exact binding epitopes of these ten Nbs. Crosslinking mass spectrometry (CL-MS) has emerged to a powerful tool for structural investigation and we have previously investigated LRRK2 by CL-MS and optimized the conditions for this protein [2]. Moreover, chemical crosslinking of the LRRK2-Nb complexes followed by sequencing of the target peptides via mass spectrometry is also a versatile method to allow sensitive mapping of Nb binding epitopes at high confidence [11,32]. The CL-MS data revealed, that the different Nbs, showed inter-protein crosslinks to LRRK2 predominantly via one conserved lysine residue in the framework 3 region of the Nb, located in the loop connecting the β-strands C″ and D [33]. This lysine residue is present in all selected Nbs except for Nb6 (CA12618), for which correspondingly no crosslinking data could be obtained. For the other Nbs, and depending on the binding epitope, this lysine residue showed robust linkage to several lysine residues within LRRK2 (FIG. 3).

Overall, the CL-MS data are in very good agreement with the results of the domain mapping using ELISA. Based on the ELISA experiments, Nbs Nb17 (CA13606), Nb36 (CA14130), Nb38 (CA14133) and Nb39 (CA14134) were flagged as “full-length LRRK2 binders”, since they only showed binding to full-length LRRK2 while no significant binding was observed to any of the tested individual domain constructs. Correspondingly, CL-MS reveals that all these Nbs make multiple contacts to the N-terminal LRR domain as well as C-terminal parts of LRRK2, including the C-terminal end of COR (CA14134), the kinase domain (CA13606 and CA14133) and both the kinase and WD40 domain (CA14130). This finding clearly demonstrates that a significant portion of the established binders indeed specifically recognize conformational epitopes instead of binding to short linear peptide stretches. Moreover, this indicates that the LRR domain “folds back” upon and is in close proximity to the C-terminal domains of LRRK2. For Nb42 (CA14259) only one crosslink with a lysine (K1502) within the Roc domain is identified in good agreement with the domain mapping in ELISA. Nb22 (CA13611), Nb23 (CA13612) and Nb40 (CA14135) were all identified as kinase-WD40 binders in ELISA, and correspondingly the crosslinking data reveals interactions with the WD40 domain, the kinase domain and both the WD40 domain and the C-terminal end of COR-B for these three Nb, respectively. Interestingly, Nb1 (CA12610) which was identified as a COR-B binder in ELISA, makes crosslinks with various lysines in different parts of the LRRK2 protein. In agreement with the ELISA data, a crosslink is found with K1833 in COR-B, but also crosslinks are found with residues within the other LRRK2 domains, including the adjacent kinase domain but also the leucine-rich repeats. This finding is in good agreement with a very central localization of the COR domain within the low-resolution structural model of the compact LRRK2 dimer [2]. We can thus hypothesize that binding of CA12610 to the COR-B domain, places it in a central cavity of the LRRK2 structure, in relatively close proximity to most other LRRK2 domains.

### Example 5. Nb Binding Affinity to LRRK2 as Determined by Microscale Thermophoresis (MST) and Bio-Layer Interferometry (BLI)

To determine the binding affinity (dissociation constant K_{D}) of the ten Nbs for LRRK2, two methods were used in parallel: microscale thermophoresis (MST) and bio-layer interferometry (BLI). For the MST experiment, we site-specifically labeled the ten Nbs with a m-TAM RA fluorophore at its C-terminus, using sortase-mediated coupling [51], and then titrated increasing amounts of full-length LRRK2 to these Nbs (FIGS. 13A-13E). For all 10 Nbs an MST signal was observed, except for Nb23, which did not generate a change in thermophoresis behavior upon binding to LRRK2. For the other Nbs KD values are found in the range of 25 nM-150 nM (Table 2). For the BLI experiment LRRK2 was first trapped on a streptavidin-coated biosensor using biotinylated Nb40 as trapping agent (except to assess binding of Nb40, where Nb42 was used as trapping agent), after which binding of all Nbs to LRRK2 was determined. A clear binding signal was obtained for all Nbs (including Nb23) with KD values ranging from 10 nM-200 nM (FIGS. 14A-14D). Overall, both methods show the same trend and affinity range, however in general a slightly higher affinity is obtained with BLI compared to MST. These differences are probably due to the experimental set-up with MST using LRRK2 in solution and BLI using LRRK2 trapped on a surface by means of a second Nb.

TABLE 2
Equilibrium dissociation constants (K_{D}) for binding of the set of
10 Nbs to LRRK2, as assessed by two methods in parallel: microscale
thermophoresis (MST) and biolayer interferometry (BLI).

| | Functional Group | | |
|---|---|---|---|
| Nanobody | (see FIG. 2d) | K_{D} (nM)-MST | K_{D} (nM)-BLI |
| Nb1 | Group 1 | 91 ± 28 | 114 ± 32 |
| Nb6 | | 83 ± 24 | 7 ± 1 |
| Nb23 | | NB^{a} | 65 ± 18 |
| Nb42 | | 94 ± 30^{b} | 82 ± 17 |
| Nb17 | Group 2 | 67 ± 24 | 11 ± 1 |
| Nb36 | | 78 ± 25 | 51 ± 10 |
| Nb38 | | 48 ± 11 | 86 ± 8 |
| Nb40 | | 26 ± 10 | 41 ± 6 |
| Nb22 | Group 3 | 145 ± 55 | 76 ± 9 |
| Nb39 | Group 4 | 79 + 19 | 21 ± 3 |

^{a}NB: no signal observed in MST
^{b}Value determined in presence of GTPgS instead of GDP, which was used in all the other measurements

### Example 6. Several LRRK2-Activity-Modulating Nbs Inhibit LRRK2 Kinase Activity In Vitro Using Different (Allosteric) Mechanisms

Next, we screened the influence of the 10 selected Nbs on the in vitro LRRK2 (wild-type) kinase activity.

Hereto, we used the fluorescence-based PhosphoSens® Protein Kinase Assay (AssayQuant Technologies Inc.), using the LRRK2-optimized AQT0615 peptide as substrate at a fixed concentration of 10 μM. The increase in fluorescence (Δ_{exc}:360 nm; Δₑₘₘ:485 nm) due to LRRK2-catalyzed peptide phosphorylation was measured continuously in time under initial rate conditions at an ATP concentration of either 0.1 and 1 mM. Subsequently, the 10 Nbs were added at a final concentration of 25 μM and the effect on the initial rate was determined in triplicate (FIG. 4A). Two negative controls, where either no Nb or an irrelevant Nb was added, were also included. Moreover, the LRRK2 specific ATP-competitive inhibitor MLi-2 [34] was added at 25 μM as a positive control. These experiments were performed in addition with LRRK2 either in the presence of a large excess (500 μM) of GDP or GTPγS (FIG. 4B), but no significant influence of the nucleotide on the inhibition profile was observed.

Consistent with the in cellulo data the WD40 domain-binding Nb22 (CA13611) activates the kinase activity of LRRK2 in vitro, with an addition of 25 μM of Nb leading to an increase in kinase activity of about 30 to 50% compared to the controls. Although, at this point, we cannot exclude that Nb22 also interacts with the kinase domain apart from the observed interaction with the WD40 domain, this observation is indicative of a regulatory interplay between the kinase and WD40 domains. This is also in good agreement to previous reports showing that a deletion of the seven C-terminal amino acids lead to a disruption of LRRK2 kinase activity [35]. Moreover, the very recently determined cryo-EM structure of the catalytic half (Roc-COR-kinase-WD40) of LRRK2 shows that an α-helix formed by the last 28 amino acids of LRRK2, following the WD40 domain, folds back upon and intimately interacts with the kinase domain [36].

In contrast, addition of the Nbs Nb17 (CA13606), Nb36 (CA14130), Nb38 (CA14133), and Nb40 (CA14135) only lead to a very mild inhibition of AQT0615 phosphorylation (initial rates ranging between 70% and 95% of the controls). This indicates that the observed inhibition of LRRK2-mediated Rab10 phosphorylation of the latter Nbs is not due to a direct effect on the kinase activity per se, as also suggested by the observation that these Nbs severely affected Rab10 phosphorylation in cells while having a less pronounced effect on autophosphorylation at position S1292, so rather specifically influencing Rab phosphorylation. One exception seems to be Nb42, which inhibited both LRRK2 autophosphorylation and Rab phosphorylation in cells, while no inhibitory effect could be observed in vitro.

Finally, three other Nbs, Nb1 (CA12610), Nb6 (CA12618) and (to a lesser extent) Nb23 (CA13612), have a very clear significant inhibiting effect of in vitro LRRK2 kinase activity. This is most pronounced for CA12610 and CA12618 that reduce the kinase activity to a level that is only 20 to 30% that of the “no Nb” and “irrelevant Nb” controls and only just above the activity of the MLi-2 control in this assay. This is consistent with the observation that all three Nbs severely inhibited both Rab10 and autophosphorylation in cells, and act as true inhibitors of the total LRRK2 kinase activity. Interesting, while CA13612 was found to bind directly on the kinase domain, CA12610 and CA12618 achieve this effect by binding to the COR-B domain.

Since we found that Nb1 (CA12610), Nb6 (CA12618) and Nb23 (CA13612) significantly inhibit LRRK2 kinase activity in vitro, we continued to perform a dose-response analysis with these three Nbs. The Nb concentration was varied from 200 or 150 μM to 0.006 μM in a two-fold serial dilution, while keeping the peptide and ATP substrate concentrations constant at 10 μM and 1 mM, respectively (FIG. 11A-C). Fitting of these dose-response curves yielded IC₅₀ values of 8±2 μM and 14±3 μM for Nb1 and Nb6, respectively, and an approximate IC₅₀ value of 65 μM for Nb23.

### Example 7. LRRK2-Inhibiting Nbs Act Via an Allosteric Mechanism

For those three Nbs (Nb1 (CA12610), Nb6 (CA12618), Nb23 (CA13612)) identified as robustly inhibiting LRRK2 autophosphorylation and Rab phosphorylation activity in cells, as well as LRRK2 kinase activity towards peptide and Rab substrates in vitro, the results suggest that these Nbs target the LRRK2 kinase activity per se. Interestingly, while the ELISA and CL-MS experiments suggested that Nb23 (CA13612) binds to the kinase domain, Nb1 (CA12610) and Nb6 (CA12618) bind the COR domain. This strongly suggests that at least the latter two Nbs act as allosteric kinase inhibitors. To further confirm this, we set out to determine the mechanism of inhibition (competitive vs. uncompetitive vs. mixed/non-competitive) of these three Nbs vis-h-vis ATP as substrate, using our peptide phosphorylation assays as output. To this end, full Michaelis-Menten curves were obtained at a fixed (probably sub-saturating) concentration of peptide substrate (10 μM) and varying concentrations of ATP (FIGS. 11A-11C). For CA12610 and CA12618, linearization of the curves using the Lineweaver-Burk plot clearly shows intersecting lines left of the Y-axis, indicative of a mixed-type inhibition. This confirms that these Nbs are not competing with ATP for binding and that they inhibit the reaction by binding on an allosteric site, which is in agreement with the ELISA and CL-MS epitope mapping data. The observation that the linearized curves cross above the X-axis also indicates that these Nbs have a preference for binding to the non-ATP-bound state of LRRK2 (lower apparent K_{ic}^{app}) over the ATP-bound form (higher apparent K_{ic}^{app}). For CA12610, a global fit of the kinetic data using a mixed inhibition model accordingly gives a K_{ic}^{app} of 16±4 μM with an α-value of 1.8±0.5, corresponding to a K_{ic}^{app} (=affinity for apo-LRRK2) of 16 μM and a K_{ic}^{app} (=affinity for ATP-bound LRRK2) of 30 μM. For CA12618, fitting on the same model yields a K_{ic}^{app} of 5±1 μM with an α-value of 1.6±0.4, corresponding a K_{ic}^{app} of 5 μM and a K_{ic}^{app} of 8 IM. For the kinase domain-binding CA13612, the linearized curves intersect closer to the Y-axis indicating a mechanism which is more ATP-competitive like. Yet, the lines do not exactly intersect on the Y-axis and a systematic decrease in the Vₘₐₓ^{app} value with increasing Nb concentration is observed, which also here indicates a mixed-inhibition mechanism. Fitting on a mixed inhibition model for CA13612 gives a K_{ic}^{app} of 9±1 μM with an α-value of 7.5±1.9, corresponding a K_{ic}^{app} of 9 μM and a K_{ic}^{app} of 66 μM. The strong preference of CA13612 for the LRRK2 apo-form over the ATP-bound form shows that, while the Nb is not completely competing for ATP binding, ATP and CA13612 binding mutually disfavour each other. This suggests that this Nb is binding close to the ATP binding pocket or that Nb23 binding forces LRRK2 in a conformation that is incompatible with ATP binding.

### Example 8. The Nbs Engage and Colocalize with LRRK2 at Endogenous Levels

Considering the high affinity binding of the 10 selected Nbs, we next tested whether those Nbs would also be able to pull-down LRRK2 at endogenous/physiological expression levels. Therefore, we turned to lysates of mouse RAW264.7 cells, which express LRRK2 at relatively high levels [52]. Interestingly, we find that all 10 Nbs efficiently pull-down LRRK2 when added to these lysates (FIG. 15), showing that (1) the Nbs are cross-reactive toward mouse LRRK2, and (2) their affinity is sufficiently high to pull-down endogenous levels of LRRK2 from cell lysates.

To test whether Nbs can trace and visualize endogenous LRRK2 in fixed cells, we generated green fluorescent protein (GFP)-Nb fusions (fluobodies) as described before [23, 53, 54]. Recently, it was shown that LRRK2 is recruited to phagosomes upon induction of phagocytosis in immune cells [55,56]. Therefore, RAW264.7 cells were transfected with these fluobodies and treated with killed yeast bioparticles “zymosan” to induce phagocytosis. Confocal imaging of fixed cells showed that LRRK2 and Nb36 and Nb42 did not colocalize with endogenous LRRK2, while LRRK2 recruitment to phagosomes was slightly detectable in cells transfected with Nb1, Nb6, Nb17 and Nb39 (FIG. 16b). Importantly, four of the tested fluobodies (Nb22, Nb23, Nb38 and Nb40) clearly colocalized with LRRK2 on zymosan-containing phagosomes (FIG. 16a), demonstrating the ability of these Nbs to trace endogenous LRRK2 within the cells.

### Example 9. Expression of LRRK2-Targeting Nanobodies does not Result in LRRK2 Relocalization to Microtubules and a Subset of these Nanobodies Inhibits MLi-2 Induced Relocalization

LRRK2 pharmacological kinase inhibitors of different structural classes induce cellular recruitment of LRRK2 to microtubules, similar to four out of five major PD-causing mutations [37-40]. The binding of LRRK2 to microtubules subsequently reduces the kinesin—and dynein-mediated transport along microtubules [36]. In order to investigate whether our ten identified LRRK2 kinase-modulating Nbs result in a similar phenotype, HEK293 cells were co-transfected with constructs coding for mScarlet-LRRK2 and GFP-Nbs. For all 10 Nbs analyzed, confocal microscopy analysis shows that LRRK2 maintained its cytoplasmic distribution 48 hrs after co-transfection with the Nbs and no relocalization to microtubules is observed, indicating that the Nbs trap LRRK2 in a different conformation compared to classical inhibitors (FIG. 5).

Next, we wanted to investigate whether the LRRK2-targeting Nbs have the ability to alter the recruitment of LRRK2 to microtubules induced by inhibition of LRRK2 kinase activity by the specific ATP-competitive inhibitor MLi-2 [41]. To do that, HEK293 cells co-transfected with mScarlet-LRRK2 and GFP-Nb constructs were treated with 1 μM of MLi-2 (FIG. 6). Interestingly, cells co-transfected with a subset of the tested Nbs showed no MLi-2-induced recruitment to microtubules, and LRRK2 cytoplasmic disruption was maintained. This rescuing effect is most prominent for the Nbs CA13606, CA13611, CA13612 and CA14135. Recent structural information has linked LRRK2 microtubule relocalization to the conformation of the kinase domain, with a closed conformation favoring LRRK2 binding to microtubules [36]. As a consequence, the commonly used ATP-competitive type I LRRK2 inhibitors trigger this effect. In addition, it was shown before that the WD40 domain is indispensable for LRRK2-microtubule association [40]. Moreover, recently it was reported that the LRRK2 decoration on microtubules involves a WD40:WD40 dimerization interface, and disruption of this protein-protein interaction abolishes the effect of MLi-2 on LRRK2 relocalization to microtubules [42]. Interestingly, our ELISA and CL-MS experiments showed that all Nbs that inhibit MLi-2 induced microtubule localization interact with either the kinase or WD40 domain. It is therefore conceivable that these Nbs trap LRRK2 either in a “kinase open” conformation or in a conformation that masks the WD40:WD40 interface.

### Example 10. The Nbs Inhibit LRRK2 Through Binding Sites Differing from Previously Described LRRK2 Kinase Inhibitors

Most currently described LRRK2 inhibitors bind directly to the ATP-binding pocket of the kinase domain. Though, physiological forms of vitamin B12 were reported to inhibit LRRK2 kinase activity by binding to a region of the kinase domain that does not overlap with the ATP binding pocket [43]. Two of the in vitro kinase activity inhibiting Nbs described herein (Nb 1 and Nb6) bind LRRK2 predominantly via interactions with the COR-B domain, thus proposing a novel mechanism of action, and thereby excluding a mechanism of inhibition similar to these previously described LRRK2 kinase inhibitors that all directly interact with the kinase domain. On the other hand, ELISA and crosslinking MS experiments showed that a third in vitro kinase activity inhibiting Nb (Nb23) binds LRRK2 via the kinase domain, although also here kinetic analysis showed that Nb23 is a mixed (non-ATP competitive) inhibitor. To verify and confirm that Nb1, Nb6 and Nb23 bind LRRK2 in a different mode than any known small molecule kinase inhibitor, thus also different that the reported vitamin B12 forms, a competition ELISA titration experiment was performed (FIGS. 17A-17C). In this experimental setup, the ELISA experiment was performed using a fixed concentration of LRRK2 coated on the bottom of the ELISA plate, and using a dilution series of either of the three Nbs ranging from 450 nM to 0.2 nM. Detection of Nb binding in ELISA is performed using their C-terminal EPEA-tag, resulting in a dose-response titration curve reflecting an apparent affinity of the Nbs. Subsequently, a repetition of the setup in the presence of either a large excess of the ATP-competitive inhibitor MLi-2 (1 μM) [34] or of the non-ATP-competitive VitB12 derivative 5′deoxyadenosylcobalamin (AdoCbl, 250 μM) [43] was performed. Additionally, as a positive control, a repetition of the setup was performed in presence of an excess (9 μm) of the corresponding untagged Nb. While a very prominent rightward shift in the titration curves is observed when adding the corresponding untagged Nb as a direct orthosteric competitor, as expected, no rightward shift is observed for any of three Nbs in presence of either MLi-2 or AdoCbl. This thus further proves that (1) neither of these three group 1 Nbs, as described herein, bind in the same pocket as the Type I ATP-competitive inhibitor MLi-2, thus confirming that these Nbs act via a non-ATP competitive allosteric inhibitory mechanism, and (2) neither of the Nbs bind to the same epitope as AdoCbl showing that they act via a completely novel allosteric mechanism.

### Example 11. Creating Multivalent and Multiparatopic Nanobodies to Increase Affinity and Potency

A panel of allosteric Nbs have been identified that bind to LRRK2 and inhibit LRRK2 kinase activity by targeting different non-overlapping epitopes on different domains. This thus holds the potential to genetically fuse combinations of such Nbs targeting different LRRK2 epitopes, resulting in multivalent/multiparatopic (i.e. binding on different epitopes of the same target) Nbs. Creating this multivalency is expected to result in significantly increased (apparent) affinities, due to both additive and cooperative (avidity) effects. Moreover, it is expected that similarly a combination of multiparatopic Nb will synergistically increase the potency of kinase inhibition in comparison to a mixture of the individual Nbs [44, 45]. This is especially true for Nbs that inhibit kinase activity by binding through different domains such as, but not restricted to, the COR-B (e.g. CA12610 and C12618) and kinase domain (e.g. CA13612).

To combine the different Nanobody building blocks, flexible (G₄S)ₓ linkers may be used with various lengths by varying the number of repeats x. Either combinations or two or more different Nbs may be made in the pMESY4 vector. Moreover, apart from varying the linker length also the order of the Nbs within the fusion may be varied. The resulting multiparatopic Nbs may be expressed and purified in a similar way as the individual Nbs. First the IC₅₀ value of the multiparatopic constructs is determined using the PhosphoSens® Protein Kinase Assay, as described above, and compared to the cocktail of the corresponding individual Nbs to detect a synergistic effect of the genetic linkage. Second, one may also determine the (apparent) K_{D} values of the multiparatopic Nbs using MST or BLI methods, as described herein, and comparing these to the K_{D} values of the individual Nbs. The most promising Nbs are subsequently tested for their effect on LRRK2 kinase activity in cells.

Materials and Methods

Protein Expression and Purification

Full-length human LRRK2 was expressed and purified based on previously developed protocols [1,2], with minor adaptations to obtain LRRK2 in a certain nucleotide-bound state: either Guanosine-5′-(γ-thio)-triphosphate (GTPγS) or Guanosine-5′-diphosphate (GDP). Briefly, full-length LRRK2 was cloned into the N-Strep/Flag-TAP (N-SF-TAP) pcDNA3.0 vector coding for a protein with an N-terminal Twin-Strep-tag and FLAG-tag (NSF) [3]. HEK 293T cells (CRL-11268; American Type Culture Collection) were transfected between 50% and 70% confluence with 8 μg of plasmid DNA/14 cm culture dish using polyethyleneimine (PEI) 25 kDa (Polysciences), and cultured post-transfection for 48 hrs in 14-cm dishes in DMEM (Sigma-Aldrich) supplemented with 10% (vol/vol) FBS (Sigma-Aldrich) and appropriate antibiotics. After removal of the medium, the cells were resuspended in lysis buffer (1 mL/14-cm dish) containing 50 mM HEPES (pH 8.0), 150 mM NaCl, 5 mM MgCl₂, 2 mM DTT, 5% Glycerol and supplemented with 0.55% (v/v) Nonidet P-40, complete protease inhibitor (Roche) and either 1 mM GTPγS or 1 mM GDP. Cell lysis was allowed to proceed for 1 h at 4° C. on a rotating shaker (10 rpm) and cell debris and nuclei were removed by centrifugation at 10,000×g for 10 min. The lysate was incubated with Strep-Tactin beads (IBA, 500 μl bed volume/15 mL cell lysate) for 2 h at 4° C. on a rotating shaker. The beads were transferred to a microspin column (GE Healthcare) and washed extensively 5 times with washing buffer (50 mM HEPES (pH 8.0), 100 mM NaCl, 1 mM DTT, 5 mM MgCl₂, 0.5 mM EDTA, 10% [vol/vol] glycerol) containing either 1 mM GTPγS or GDP. Elution was performed with 700-800 μL of the same washing buffer containing 2.5 mM of D-Desthiobiotin (IBA) and 1 mM of either GTPγS or GDP.

A domain construct of LRRK2 spanning the Roc and COR domains (RocCOR) was expressed initially from a pBADcLIC vector coding for a protein spanning the a.a. residues 1334-1840 fused to a N-terminal Twin-Strep-tag and C-terminal Hisio-tag. In a later stage a construct spanning residues 1293-1840 cloned in a pDEST-566 vector and coding for a protein with an N-terminal His₆-MBP (maltose-binding protein)-tag was used. While the protein expressed from the pBADcLIC vector was used for the immunization and most screening experiments, the protein expressed from the pDEST-566 vector was used also in certain screening experiments. Expression and purification of the His₆-MBP-tagged RocCOR was performed similar to the purification of the COR-B construct (see further). The pBADcLIC vector containing the RocCOR coding region was transformed into an E. coli strain (E. coli RCEv9) that was custom evolved in-house starting from a MC1061ΔacrB strain for optimal expression of the RocCOR protein, using previously described protocols [4,5]. An overnight culture was used to inoculate 4 L of TB medium (37° C.), and when an OD of about 0.7 was reached protein expression was induced with 0.01% of arabinose and allowed to proceed overnight at 20° C. Cells were harvested and resuspended into a buffer containing 50 mM Tris-HCl pH 7.5, 500 mM NaCl, 10 mM MgCl₂, 2 mM β-mercaptoethanol and 20 mM imidazole and supplemented with 1 mM of PMSF, 1 μg/mL of Leupeptin, 0.1 μg/mL of AEBSF and 50 μg/mL of DNaseI. Finally, either 0.5 mM GDP or 0.5 mM Guanosine-5′-[(β,γ)-imido]triphosphate (GppNHp) was added to the buffer prior to cell lysis. Cells were lysed using a Cell Disrupter (Constant Systems Ltd.) and after clearance via centrifugation the cell lysate was loaded on a 5 mL Ni-NTA column. First, the column matrix was washed with 10 column volumes (CV) of resuspension buffer supplemented with 300 mM KCl and 5 mM ATP to reduce contamination with chaperones. Subsequently, the column was washed with 10 CV of a buffer containing 50 mM Tris-HCl pH 7.5, 150 mM NaCl, 10 mM MgCl₂, 20 mM imidazole, 5% glycerol, 2 mM β-mercaptoethanol and either 0.5 mM GDP or GppNHp, and the proteins were eluted in the same buffer supplied with 300 mM imidazole. After a concentration step, a final purification step consisted of a gel filtration on a Superdex S200 10/300 column using 30 mM HEPES pH 7.5, 150 mM NaCl, 5 mM MgCl₂, 5% glycerol, 1 mM DTT as a buffer supplemented with either 0.5 mM GDP or GppNHp.

A construct of the Roc domain spanning the residues 1329-1520 was cloned in the pET-28a vector providing an N-terminal His₆-tag, and the vector was transformed in the E. coli BL21(DE3) strain. An overnight culture was used to inoculate 4 L of TB medium (37° C.), and when an OD of about 0.7 was reached protein expression was induced with 0.1 mM isopropyl β-D-1-thiogalactopyranoside (IPTG) and allowed to proceed overnight at 20° C. Cells were harvested and resuspended into a buffer containing 30 mM HEPES pH 7.5, 250 mM NaCl, 10 mM MgCl₂, 10 mM glycine and 20 mM imidazole and supplemented with 1 mM of PMSF, 1 μg/mL of Leupeptin, 0.1 μg/mL of AEBSF and 50 μg/mL of DNaseI. Cells were lysed using a Cell Disrupter (Constant Systems Ltd.) and after clearance via centrifugation, the cell lysate was loaded on a 5 mL Ni-NTA column. After extensive washing with 10 CV of the resuspension buffer, proteins were eluted in the same buffer containing 300 mM imidazole. A final purification step consisted of a gel filtration on a Superdex S75 10/300 column using 30 mM HEPES pH 7.5, 150 mM NaCl, 5 mM MgCl₂, 5% glycerol, 1 mM DTT as buffer.

A construct of the C-terminal part of the COR domain (COR-B) spanning the residues 1672-1840 was cloned in the pDEST-566 vector providing an N-terminal His₆-MBP-tag, and the vector was transformed in the E. coli BL21(DE3) strain. An overnight culture was used to inoculate 4 L of TB medium (37° C.), and when an OD of about 0.7 was reached protein expression was induced with 0.5 mM IPTG and allowed to proceed for 2 h at 20° C. Cells were harvested and resuspended into a buffer containing 30 mM HEPES pH 7.5, 200 mM NaCl, 1 mM EDTA and 1 mM DTT and supplemented with 1 mM of PMSF, 1 μg/mL of Leupeptin, 0.1 μg/mL of AEBSF and 50 μg/mL of DNaseI. Cells were lysed using a Cell Disrupter (Constant Systems Ltd.) and after clearance via centrifugation, the cell lysate was loaded on a 5 mL MBPTrap column (GE Healthcare). After washing with 10 CV of resuspension buffer, the protein was eluted in the same buffer containing 10 mM maltose. A final purification step consisted of a gel filtration on a Superdex S200 10/300 column using 30 mM HEPES pH 7.5, 150 mM NaCl as buffer.

A domain construct of LRRK2 containing the kinase and WD40 domains (K-WD40) and spanning the residues 1876-2527 was cloned in a pFastBac vector (Invitrogen) encoding a protein with an N-terminal His-tag. The protein was expressed in Sf9 cells (Invitrogen) and purified by affinity chromatography using a Ni-NTA matrix as previously described [6]. A final purification step consisted of a gel filtration on a Superdex S75 10/300 column using 50 mM Tris-HCl pH 7.5, 150 mM NaCl, 5 mM MgCl₂, 2 mM 3-mercaptoethanol and 0.1 mM GDP as buffer.

Nanobody (Nb) expression and purification was performed as described previously [7]. The Nb-coding open reading frames, cloned in the pMESy4 vector (see further), were transformed in non-suppressor E. coli WK6 (Su⁻) cells. Cells were grown at 37° C. in Terrific Broth (TB) medium and protein expression was induced with 1 mM IPTG. After overnight expression at 28° C., cells were harvested via centrifugation and subjected to an osmotic shock to obtain the periplasmic extract. Subsequently, an affinity purification step on Ni²⁺-NTA Sepharose followed by a dialysis step against a buffer consisting of 20 mM Tris-HCl pH 7.5, 150 mM NaCl was used to purify the Nbs.

Immunizations

In total three llama immunizations were performed: (1) with the RocCOR domain construct of human LRRK2; (2) with full-length LRRK2 in the presence of GTPγS; and (3) with full-length LRRK2 in the presence of GDP. To obtain LRRK2 in a particular nucleotide-bound state (GDP versus GTPγS), all purification steps were performed in presence of an excess of the respective nucleotide (see above). Moreover, to assure that the protein remains in the particular nucleotide-bound state upon and after immunization, a mild crosslinking was performed on the protein prior to immunization. Therefore, LRRK2 protein either loaded with 1 mM GTPgS or 1 mM GDP was incubated with the primary amine-specific crosslinker disuccinimidyl suberate (DSS) in 1:20 molar ratio for 30 minutes, after which the reaction was quenched by adding an excess of Tris.

For all three independent immunization strategies, a six-week protocol with weekly immunizations in presence of GERBU adjuvant was followed. All animal vaccinations were performed in strict accordance with good practices and EU animal welfare legislation. In immunization strategy (1) the RocCOR domain construct in presence of 10 mM GppNHp was used for immunization. 200 μg of protein was injected in the first 2 weeks and 100 μg in the last two weeks. Immunization with full-length (partially crosslinked) LRRK2 in presence of 10 mM GTPγS (immunization (2)) or 10 mM GDP (immunization (3)) were performed according to the following scheme: 300 μg protein injected in week 1, 200 μg protein injected in week 2, 100 μg protein injected in weeks 3-6. Blood was collected 4 days after the last injection.

Nanobody Generation

Construction of immune libraries and Nb selection via phage display were performed using previously described protocols [7] with modifications to maximize chances to select Nbs that bind specifically to different nucleotide forms of LRRK2. In brief, starting from the blood collected from the llamas after immunization with respectively the RocCOR domain (immunization 1), LRRK2 in presence of GTPγS (immunization 2) and LRRK2 in presence of GDP (immunization 3), the variable domains of the heavy-chain antibody repertoire were cloned in a pMESy4 phage display vector, which adds a C-terminal His₆-tag and EPEA-tag (=CaptureSelect™ C-tag) upon protein expression. This resulted in three independent immune libraries of 8.3×10⁸, 1.8×10⁹ and 1.3×10⁹ transformants, respectively. This Nb repertoire was expressed on the tip of filamentous phages after rescue with the VCSM13 helper phage. For immunization 1, two consecutive rounds of phage display selection were performed against either solid phase coated full-length LRRK2 or full-length LRRK2 trapped on beads via anti-flag M2 Ab (Merck). Coating of LRRK2 was performed in a coating buffer containing 50 mM HEPES pH 8.0, 150 mM NaCl, 5 mM MgCl₂, 5% glycerol, supplemented with 100 μM of GDP and blocking was performed with 2% BSA. All the binding and washing steps were performed in a washing buffer containing 50 mM HEPES pH 8.0, 150 mM NaCl, 5 mM MgCl₂, 5% glycerol and 0.05% Tween20, supplemented with 100 μM GDP. For immunization 2, two consecutive rounds of phage display selection were performed using solid phase coated full-length LRRK2. Additionally, three consecutive rounds of phage display selection were performed using the solid phase coated LRRK2 Roc domain. During the coating steps the coating buffer was supplemented with 1 mM GTPγS, while during binding and washing steps the washing buffer was supplemented with 100 μM GTPγS. For immunization 3, one round of phage display selection was performed using solid phase coated full-length LRRK2. Additionally, two consecutive rounds of phage display selection were performed using the solid phase coated LRRK2 Roc domain. During the coating steps the coating buffer was supplemented with 1 mM GDP, while during binding and washing steps the washing buffer was supplemented with 100 μM GDP. Several single colonies were picked after each round of phage display selection and sequence analysis was used to classify the resulting Nb clones in sequence families based on their CDR3 sequence.

In order to allow expression of the Nbs as fluorescently (eGFP)-labeled intrabodies (Fluobodies) in HEK 293T cells, the Nb ORFs were recloned to the pEGFP-N1 vector using the HindIII and BamHI restriction sites. This will result in expression of the Nbs fused at their C-terminus to eGFP (Nb-GFP). To allow expression of the Nbs in HEK 293T cells without GFP, a stop codon was introduced between the Nb- and GFP-coding sequence.

ELISA Experiments

Before Nb purification, binding of the Nbs to LRRK2 was confirmed using an ELISA screen on crude extracts of E. coli cells expressing the respective Nbs. Full-length LRRK2 was solid phase coated on the bottom of the ELISA well, and the coating, blocking, binding and washing buffer were kept the same as in the phage display experiments and were supplemented with the relevant nucleotides (GTPγS or GDP). Binding of the Nbs to LRRK2 was detected via their EPEA-tag using a 1:4000 CaptureSelect™ Biotin anti-C-tag conjugate (Thermo Fischer Scientific) in combination with 1:1000 Streptavidin Alkaline Phosphatase (Promega). Color was developed by adding 100 μl of a 4 mg/mL disodium 4-nitrophenyl phosphate solution (DNPP, Sigma-Aldrich) and measured at 405 nm.

After purification of the selected Nbs as described above, ELISA experiments were performed to determine their domain specificity. Full-length LRRK2 and the Roc, COR-B, RocCOR and K-WD40 domain constructs were solid phase coated in 96-well ELISA plates. The coating, binding and washing buffer were kept the same as in the phage display experiments, supplemented with 100 μM GDP. The ELISA was developed in the same way as described above.

In Cellulo Phospho-Rab Assay

HEK 293T cells were cultured in DMEM (supplemented with 10% Fetal Bovine Serum, 25 mM L-Glutamine and 0.5% Pen/Strep). For the assay, the cells were seeded onto six-well plates and transfected at a confluency of 50-70% with the individual Nb-GFP expression constructs, SF-tagged LRRK2(G2019S) and FLAG-HA Rab29 using a self-made polyethylenimine (PEI)-based transfection reagent [8]. After 48 hrs cells were lysed in lysis buffer [30 mM Tris-HCL (pH7.4), 150 mM NaCl, 0.5% Nonident-P40, complete protease inhibitor cocktail, phosphatase inhibitor cocktail II & III (all Sigma)]. Lysates were cleared by centrifugation at 10,000×g and adjusted to a protein concentration of 1 μg/μl in 1× Laemmli Buffer. Samples were subsequently subjected to SDS PAGE and Western Blot analysis to determine LRRK2 pS1292 and Rab10 T72 phosphorylation levels, as described below. Total LRRK2 and Rab10 levels were determined as a reference.

For western blot analysis, protein samples were separated by SDS-PAGE using NuPAGE 10% Bis-Tris gels (Invitrogen) and transferred onto PVDF membranes (Thermo Fisher). To allow simultaneous probing for LRRK2 on the one hand and Rab and the Nb-GFP fusions on the other hand, membranes were cut horizontally at the 140 kDa MW marker band. After blocking non-specific binding sites with 5% non-fat dry milk in TBST (1 h, RT) (25 mM Tris, pH 7.4, 150 mM NaCl, 0.1% Tween-20), membranes were incubated overnight at 4° C. with primary antibodies at dilutions specified below. Phospho-specific antibodies were diluted in TBST/5% BSA (Roth GmbH). Non-phospho-specific antibodies were diluted in TBST/5% non-fat dry milk powder (BioRad). Phospho-Rab10 levels were determined by the site-specific rabbit monoclonal antibody anti-pRAB10(pT73) (Abcam, ab230261) and LRRK2 autophosphorylation was determined by the site-specific rabbit monoclonal antibody anti-pLRRK2(pS1292) (Abcam, ab203181), both at a dilution of 1:2,000. Total LRRK2 levels were determined by the in-house rat monoclonal antibody anti-pan-LRRK2 (clone 24D8; 1:10,000) [9]. Total Rab10 levels were determined by the rabbit monoclonal antibody anti-RAB10/ERP13424 (Abcam, ab181367) at a dilution of 1:5,000. Nb-GFP fusion proteins were detected using the rat monoclonal antibody anti-GFP (clone 3H9, ChromoTec) at a dilution of 1:2,000. For detection, goat anti-rat IgG or anti-rabbit IgG HRP-coupled secondary antibodies (Jackson ImmunoResearch) were used at a dilution of 1:15,000 in TBST/5% non-fat dry milk powder. Antibody-antigen complexes were visualized using the ECL plus chemiluminescence detection system (GE Healthcare) on Hyperfilms (GE Healthcare).

Chemical Crosslinking/Mass Spectrometry (CL-MS)

For chemical crosslinking, the LRRK2 protein solution was adjusted to a concentration of 3 μM (0.86 mg/mL). After extensive dialysis to remove Tris buffer, each nanobody was added to the purified LRRK2 at a final molar ratio of 2:1 in the LRRK2 elution buffer (see section protein purification) To allow complex formation, the protein mixture was incubated for 1 h at 4° C. under constant mixing. The crosslinking reaction was then performed using the NHS-ester-based and CID-cleavable reagent disuccinimidyl sulfoxide (DSSO; Thermo Fisher) [10] at a molar excess of 60:1 (referred to the nanobodies). The crosslinking reaction was carried out for 30 min at room temperature under constant mixing. The reaction was then stopped by adding Tris-HCl (pH 7.5) solution to a final concentration of 10 mM and incubation of 15 min at room temperature. Proteins were finally precipitated by chloroform/methanol and subsequently subjected to tryptic proteolysis as described in [8]. The tryptic peptide solutions were cleaned up by StageTips and subjected to SEC separation to enrich for crosslinked peptides as described earlier [2]. Vacuum-dried fractions containing the crosslinked peptides, were analyzed individually on an Orbitrap Fusion mass spectrometer (Thermo Fisher) using the MS2_MS3 fragmentation method with the default settings (ver. 3.0, build 2041). MS1 scans were performed in the Orbitrap (FTMS, resolution=60K) at an m/z range of 375-1500. MS2 was performed with CID (CE=25%) and spectra were acquired in the Orbitap (FTMS) at 30K resolution. The MS3 scans were performed with HCD (CE=30%) and spectra were acquired in the linear ion trap. The Thermo Raw files were analyzed with the MS2_MS3 workflow provided by in Proteome Discoverer 2.4, which uses XlinkX (ver. 2.4) [11] for the detection of crosslinked peptides. Briefly, a global search of MS2 spectra was performed with Sequest HT against the human subset of the Swissprot database (v. 2019_02; 20417 entries) supplemented with the sequences of the nanobodies followed by an FDR analysis (FDR=0.01) by the Target Decoy PSM validator. For the Sequest analysis, the following settings have been used: Trypsin has been used as enzyme. Carbamylation of cysteines has been used as fixed modification and Methionine oxidation, DSSO hydrolyzed (K+176.014 Da), DSSO Tris (K+279.078 Da) and n-terminal Acetylation were allowed as variable modifications.

For the detection of crosslinked peptides by the XlinkX detection node, the acquisition strategy was set to MS2_MS3 and DSSO (158.004; K) was used as crosslinker at an S/N minimum of 1.5. For the XlinkX database search, the following parameters have been used: Trypsin has been used as enzyme. The precursor and fragment mass tolerances were set to 10 ppm (precursor), 20 ppm (FTMS) and 0.5 Da (ITMS), respectively. The search was performed using a database containing the LRRK2 sequence and the individual sequences of all used nanobodies. Carbamidometyl has been used as fixed and Methionine oxidation was allowed as variable modification. FDR-based analysis (XlinkX validator node) was performed using the Perculator setting with a FDR threshold of 0.01.

In the consensus step, the identified crosslinks were filtered for an identification score ≥20 (default value) to reduce the number of false-positive hits. Filtered crosslinking data were exported and visualized in xiNet [12].

In Vitro Peptide Phosphorylation (Kinase) Assay

The influence of the purified Nbs on LRRK2 kinase activity was determined using the PhosphoSens® Protein Kinase Assay (AssayQuant Technologies Inc.) using the optimized LRRK2 AQT0615 peptide as substrate and according to the manufacturers' instructions. The continuous kinase assay was performed in a total volume of 50 μL in a black half area 96 well plate. Each reaction mixture contained 10 μM AQT0615 peptide substrate/probe, either 0.1 mM or 1 mM ATP and 500 μM of either GDP, or GTPγS in a buffer consisting of 50 mM HEPES pH 7.5, 0.1% Brij-35, 50 mM NaCl and 10 mM MgCl₂. The reaction was initiated by addition of a final concentration of 80 nM of LRRK2 either in absence or presence of 25 μM Nb. Prior to addition, LRRK2 and the Nbs were pre-incubated for 30 minutes on ice. The LRRK2-catalyzed phosphorylation of the peptide substrate/probe is followed continuously at 30° C. in a plate reader with excitation and emission wavelength of 360 nm and 485 nm respectively. Time traces were corrected by subtracting the “no LRRK2” control. The initial rates were determined from the slope of the linear portion of the curve.

To determine IC₅₀ values the Nb concentration was varied from 150 μM to 0.006 μM using a two-fold serial dilution. In these assays a final LRRK2 concentration of 150 nM and an ATP concentration of 1 mM was used. The relative LRRK2 activity (compared to the “no nanobody” control) was plotted against the logarithmic Nb concentration and fitted on a three-parameter log(inhibitor) vs response equation in the GraphPad Prism software. All time traces were collected in triplicate.

Confocal Microscopy and Microtubule Localization

A HEK293 cell line was cultured in complete media (high-glucose Dulbecco's modified Eagle's medium, 10% fetal bovine serum and Penicillin-Streptomycin-Glutamine (Gibco)). Cells were seeded on 8-well p-Slide(Ibidi) and transfected at a confluency of 50-70% with GFP-Nb and mScarlet-LRRK2 constructs, using JetPEI reagent (Polyplus transfection). After 24 hrs, cells were treated with either DMSO or 1 μM of LRRK2 kinase inhibitor (MLi-2, cat. no. 5756, TOCRIS) for 90 mins and then examined for localization. Data acquisition was done with a ×100 oil-immersion objective with a Zeiss LSM800 confocal laser scanning microscope. Image analysis of z-scan was done using the Zeiss microscope software ZEN.

Determination of Kinase Inhibition Mechanism

To determine the mechanism of inhibition and apparent Kᵢ (Kᵢ^{app}) values of CA12610, CA12618, CA13612 the PhosphoSens® Protein Kinase Assay (AssayQuant Technologies Inc.) in combination with the AQT0615 peptide substrate was used. Full Michaelis-Menten curves (relative velocity versus [ATP]) in presence of different Nb concentrations were collected at 30° C. in a buffer consisting of 50 mM HEPES pH 7.5, 0.1% Brij-35, 50 mM NaCl, 10 mM MgCl₂ and 500 mM GDP, using a fixed concentration of AQT0615 (10 μM) and varying concentrations of ATP. The LRRK2 concentration was chosen such that initial rates (linear fluorescence versus time curves) were obtained, and LRRK2, ATP and the Nbs were preincubated at 4° C. for 30 min prior to starting the reaction by adding the peptide substrate. The Michaelis-Menten curves for the different Nb concentrations were globally fit on a mixed-inhibition model using GraphPad Prism, according to the equation:

$$v = \frac{\frac{{V}_{m a x}^{app}}{( 1 + \frac{[ Nb ]}{α . {K}_{i}^{app}} )} . a}{{K}_{M}^{app} . \frac{( 1 + \frac{[ Nb ]}{{K}_{i}^{app}} )}{( 1 + \frac{[ Nb ]}{α . {K}_{i}^{app}} )} + a}$$

(with K_{ic}^{app}=Kᵢ^{app} and Kᵢ^{app}−a·Kᵢ^{app})

Additionally, as a diagnostic tool, Michaelis-Menten curves were linearized using the Lineweaver-Burk (double reciprocal) plot.

Pull-Down Experiments

Fresh lysate of HEK293 cells overexpressing SF-tagged LRRK2 and GFP-tagged Nbs was prepared in 100 μL ice-cold lysis buffer (10 mM Tris/HCl pH 7.5, 150 mM NaCl, 0.5 mM EDTA, 0.5% NP-40), containing complete EDTA-free protease inhibitor cocktail (Sigma-Aldrich Cat #11836170001) and Protease Inhibitor Cocktail (Sigma, cat. no. P-2714). GFP-Nbs were immunoprecipitated with Magnetic GFP nanotrap beads (ChromoTek). Immune complexes were washed twice with 10 mM Tris/HCl pH 7.5 and subjected to immunoblot analysis by boiling samples in sample buffer with a reducing agent. Samples were separated on 4-15% Tris-Glycine gels (Mini-PROTEAN TGX™ Precast Gels, Bio-rad), transferred onto a nitrocellulose membrane (GE Lifesciences), and processed for western analysis. Membranes were blocked in 5% dry milk in Tris-buffered saline plus Tween-20 for 1 hour. To allow for separate detection of LRRK2 and Nbs, the membrane was cut horizontally at 180 kDa and the upper part was probed with rat monoclonal anti-LRRK2 (clone 24D8 1:1000, Gloeckner lab), while the lower part was probed with Rabbit anti-GFP antibodies, 1:2500 (MA5-15256, Invitrogen) and incubated overnight at 4 C with gentle shaking. Membranes were then washed three times for 10 min at room temperature in PBS containing 0.1% or 0.05% Tween-20 and then incubated for 1 hour with anti-rat IgG-HRP (sc-2750, Santa Cruz Biotechnology) for LRRK2 and anti-rabbit HRP conjugated (#7074, Cell Signaling, 1:5000) for GFP-Nbs. Membranes were again washed three times for 10 min at room temperature in PBS containing 0.1% or 0.05 Tween-20. The membranes were coated with enhanced chemiluminescent (ECL) reagent (WesternSure PREMIUM, Li-COR biosciences), and proteins were detected using the C-Digit Imaging System (Li-COR Biosciences)

To test for binding of Nbs with endogenous (mouse) LRRK2, fresh lysates of RAW264.7 cells were prepared in 10 mM Tris/HCl pH 7.5; 150 mM NaCl; 0.5% NP-40 supplemented with complete EDTA-free protease inhibitor cocktail (Sigma-Aldrich Cat #11836170001) and Protease Inhibitor Cocktail (Sigma, cat. no. P-2714). Purified His-tagged Nbs were added to the lysate at a final concentration of 1.5 mM and the mixture was allowed to rotate at 4° C. overnight. His-tagged Nbs were pulled down by magnetic Dynabeads (Invitrogen). Immune complexes were washed twice with 10 mM Tris/HCl pH 7.5 and subjected to immunoblot analysis in a similar way as described above. To allow for separate detection of LRRK2 and Nbs, the membrane was cut horizontally at 180 kDa and the upper part was probed with rabbit monoclonal anti-LRRK2, 1:1000 ([MJFF2 (c41-2)], ab133474, Abcam) while the lower part was probed with mouse anti-Histidine Tag antibody, 1:1000 (AD1.1.10, Bio-rad) and incubated overnight at 4° C. with gentle shaking. Membranes were then washed three times for 10 min at room temperature in PBS containing 0.1% or 0.05% Tween 20 and then incubated for at least 1 hour with secondary antibodies: anti-rabbit HRP-conjugated (#7074, Cell Signaling, 1:500) or mouse IgG kappa binding protein (m-IgGk BP) conjugated to HRP (sc-516102, Santa Cruz Biotechnology, 1:5000). Membranes were again washed three times for 10 min at room temperature in PBS containing 0.1% or 0.05% Tween-20. The membranes were coated with enhanced chemiluminescent (ECL) reagent (WesternSure PREMIUM, Li-COR biosciences), and proteins were detected by C-Digit Imaging System (Li-COR Biosciences).

Microscale Thermophoresis and Bio-Layer Interferometry Measurements.

Microscale thermophoresis (MST) experiments were performed for determining equilibrium binding affinities (K_{D}) of Nbs binding to FL-LRRK2. Therefore, Nb were site-specifically labelled at their C-terminus with m-TAMRA, using Sortase-mediated peptide exchange [57]. The Nbs were recloned into a pHEN29 vector which is subsequently used to express and purify the Nbs with a C-terminal LPETGG-His₆-EPEA tag. Exchange of the latter peptide with an m-TAMRA-labeled GGGYK peptide (GenicBio, Shanghai, China) was performed using Sortase, and unlabeled Nb and unincorporated peptide were removed using a Ni-NTA and SEC purification step, respectively. MST measurements were performed using a Monolith NT.115 instrument (Nanotemper technologies) by titrating a fixed concentration of m-TAMRA-labelled Nb (50-100 nM depending on the affinity of the Nb) with varying concentrations of LRRK2 (16 points, 3:1 dilution series). Experiments were performed in 50 mM HEPES pH8.0, 150 mM NaCl, 10 mM MgCl₂, 5% Glycerol, 0.1% BSA, 0.05% Tween and 500 μM GDP (except for Nb42 where 500 μM GTPγS was used). After incubation at 4° C. for 30 mins, samples were loaded in capillaries and measurements were performed at 25° C. using 50-70% LED power and 80% laser intensity (laser on-time: 30 s, laser off-time: 5 s). All experiments were performed in triplicate. Data was initially processed using the MO.affinity analysis software and final K_{D} values were obtained by fitting the MST signal (at 5-15 s on-time) versus [LRRK2] curves to a quadratic binding isotherm in GraphPad Prism 7.

Biolayer interferometry (BLI) measurements were performed using an Octet Red96 (Forte Bio, Inc.) system in a buffer containing 50 mM HEPES pH 8.0, 150 mM NaCl, 10 mM MgCl₂, 5% glycerol, 0.1% BSA, 0.05% Tween, at 25° C. and with shaking speed 1000 rpm. Binding of Nbs to FL-LRRK2 were performed by first trapping LRRK2 on Streptavidin-coated (SA) biosensors by means of a high-affinity LRRK2-specific biotinylated Nb (either Nb40 or Nb42). The biotinylated Nbs were first loaded on pre-equilibrated SA biosensors at a concentration of 5 μg/mL. This Nb-loaded sensor was then used to trap FL-LRRK2 from a 50 nM LRRK2 solution. Finally, this sensor was used to monitor association (600 s to 1000 s) and dissociation (600 s to 1000 s) of the whole set of Nbs (to assess binding of Nb40 a sensor with Nb42 as trapping agent was used, for all other Nbs Nb40 was used as trapping agent). All experiments were performed in triplicate. The equilibrium dissociation constants (K_{D}) were obtained by fitting the dose-response curves, resulting from plotting the association signal at 100 s to 800 s (depending on the association rate of the Nb) versus the Nb concentration on a Langmuir equation in GraphPad Prism 7.

Immunostaining

RAW264.7 cells (ATCC® SC-6003™) were transfected with peGFP-Nb constructs using JetOPTIMUS (Polyplus transfection) for 24 hrs and then stimulated with Zymosan particles (Sigma Aldrich) for 30 mins (50 mg/mL). Immunostaining for LRRK2 was performed as reported before [78]. Briefly, cells fixed with 4% (w/v) paraformaldehyde for 30 min, followed by treatment with 100% EtOH at −20° C. Sampled were permeabilized with PBS/0.5% Triton X-100 and blocked with 3% BSA. Primary antibodies (rabbit anti-LRRK2, c41-2, Abcam and mouse anti-GFP, G6539, Sigma Aldrich) were diluted in PBS containing 3% BSA and 0.5% Triton X-100 and incubated overnight at 4° C. After three 5-min washes with PBS, secondary fluorescently labeled antibodies (anti-rabbit-Alexa fluor 568 and anti-mouse-Alexa fluor 488, Invitrogen) were added in a similar manner to primary antibodies and incubated for 1 hour at room temperature. Cells were washed twice with 1×PBS and mounted with Fluroshield antifade reagent with DAPI (SigmaAldrich). Images were acquired on Zeiss LSM800 confocal laser scanning microscope. Image analysis of z-scan was done using the Zeiss microscope software ZEN.

Competition ELISA

To assess any potential competition between the binding of Nanobodies Nb1, Nb6 or Nb23 and either Mli-2 or 5′deoxyadenosylcobalamin (AdoCbl) a competition ELISA experiment was performed. The ELISA experiment was performed similar as described above, using a fixed concentration of LRRK2 coated on the bottom of the ELISA plate, and using a 1:4 dilution series of Nb1, Nb6 or Nb23 ranging from 450 nM to 0.2 nM. Detection of Nb binding was performed using the C-terminal EPEA tag of the Nbs as described before, resulting in a dose response titration curve reflecting an apparent affinity of the Nbs. This setup were performed either in the absence or in the presence of either a large excess of the ATP-competitive inhibitor MLi-2 (1 μM), the non-ATP-competitive VitB12 derivative 5′deoxyadenosylcobalamin (AdoCbl, 250 μM), or an excess of the corresponding non-tag Nb (9 μm) in each dilution series. A “no antigen control”, where no LRRK2 was coated on the bottom of the well, was also included. The absorbance signal (A405 nm) was read out at different time points and the A405 nm at 0.5 h (Nb6 and Nb23) or 10 h (Nb1) was plotted in function of the Nb concentration. Measurements in presence of Mli-2 and AdoCbl were done in triplicate (except Nb1 in presence of AdoCbl, which was done in duplicate).

Sequence Listing

SEQ ID NO: 1-19: LRRK2 Nanobody sequences (without 6×His/EPEA C-terminal tag) (Table 3)
SEQ ID NO: 20: 6×Histidine and EPEA C-terminal tag
SEQ ID NO: 21: human Leucine-rich repeat kinase 2 amino acid sequence (T1647S mutant of Q17RV3_UniProt as used herein; 2527aa)
SEQ ID NO: 22: N-terminal SF tag (flag and twin-strep) used for human LRRK2 expression of SEQ ID NO:21 (without N-terminal Met).

The cDNA as used herein for encoding human LRRK2 was first described in [50] and the construct including the N-terminal SF-tag (SEQ ID NO:22) was first described in [1].
SEQ ID NOs: 23-79: CDR1, CDR2, and CDR3 sequences for the Nbs of SEQ ID NO:1-19 as annotated in Table 3, and as provided in Table 4.

TABLE 3
LRRK2-specific Nanobody sequences.
Nanobody as provided in the examples contains the corresponding sequence of SEQ ID NO: 1-19 and a
C-terminal 6xHis/EPEA tag. CDR annotation is labelled herein according to current analysis (+Table 4)
(see also FIG. 10 for alternative annotations).

| SEQ ID | | |
|---|---|---|
| NO: | Nbs | CDR1 CDR2 CDR3 |
| 1 | CA12610 | MAQVQLVESGGGLVQAGGSLRLSCVASGRIFSGNAMGWYRQAPGRQRELVAGITNGGYTNYRQFVKGRFTISRDNAKRTVYLQMNSLKPEDTAVYYCNALDREARQVDYWGQGTQVTVSS |
| 2 | CA12618 | MAQVQLVESGGGLVQAGGSLRLSCAASGSIFSPTAMGWYRQAPGKERELVAGITSGGYTNYRNFARGRFTISRDNAKKTVYLQMNSLNPEDTAVYYCNTLTISWSWGQGTQVTVSS |
| 3 | CA13612 | MAQVQLVESGGGLVQAGGSLRLSCAASGRTFSRYTMGWFRQAPGKDREFVARITTSGAVTTYADSAKGRFTISRDNAKNTVYLQMNTLKPEDTAVYYCAAGQGQLSSQSRFVDYWGQGTQVTVSS |
| 4 | CA14135 | MAQVQLVESGGGLVQPGGSLRLSCVASGTIYAINRMGWYRQAPGKQRELVALITLGGGTNYGDSVKGRFTISRDNAKNTGYLQMNSLKPEDTAVYYCKVESSGYTDVFWGQGTQVTVSS |
| 5 | CA13606 | MAQVQLVESGGGLVQPGGSLRLSCAASGSNIGYMAWYRQAPGSQRELVARIASGGSANYADSVKGRFTISRDNVKNTVYLHMNSLKPVDTAVYYCHADAWYNSRWSDYWGQGTQVTVSS |
| 6 | CA14259 | MAQVQLVESGGGLVQAGGSLRLSCAASGDVNYAVGWFRQAPRKEREFVAAINWRGDNTDYTDSVKGRFTISRDNAKNTVYLQMNSLKPEDTAVYYCAAKSAPYWYSDIAPDYWGQGTQVTVSS |
| 7 | CA14130 | MAQVQLVESGGGLVQPGGSLRLSCAASGSIRSITTSAWFRQPPGKQRELVAGISSGTSTNYADSVKGRFTISRDNAKNTVYLQMNSLKPEDTAVYYCHVWISGTWYSGQGTQVTVSS |
| 8 | CA14133 | MAQVQLVESGGGLVQPGGSLRLSCAASGSIRSIATSAWYRQPPGKQRELVAGISSGTTTNYADSVKGRFTISRDNAKNTVYLQMNSLKPEDTAVYYCHAWISGTWISGQGTQVTVSS |
| 9 | CA13611 | MAQVQLVESGGGLVQAGGSLRLSCAASGRTFSSYTMGWFRQAPGKEREFVAAIRWSGGSTYYADSVKGRFTISRDNAKNTVYLQMNSLKPEDTAVYYCAASDRGGFIATSGGWMDYWGKGTQVTVSS |
| 10 | CA14134 | MAQVQLVESGGGLVQAGGSLRLSCRASGGTFSTYAMGWFRQVPGKERETVAAVKWSGDMTYYADSVKGRFTISRDNAKNTVYLQMNSLRPEDTAVYYCAARRGLSYYRGPSEYDYWGQGTQVTVSS |
| 11 | CA12614 | MAQVQLVESGGGLVQPGGSLRLSCAASGSIFRRNAMGWYRQVTGKERELVAGITSGGYTNYRDFVKGRFTISRDNAKNTVYLQMNNLKPEDTAVYYCNLLSEMLQTVWGQGTQVTVSS |
| 12 | CA14136 | MAQVQLVESGGGLVQAGGSLRLSCEASVRTFSFYSMGWFRQAPGKEREFVAAIRWTAGSTSYADIVKGRFTISRDNGKNTVVLQMNSLEPEDTGVYYCAAQRGYLVEAASREYDYWGQGTQVTVSS |
| 13 | CA13620 | MAQVQLVESGGGLVQAGGSLRLSCATSGLTFGTYAMAWFRQAAEKEREFVAFISGRGGYRDYADSVKGRLTIFRDNAKNNAKNTVYLQMNNLKPEDTAVYYCAARQGSPVRSKEEYNNWGQGTQVTVSS |
| 14 | CA14131 | MAQVQLVESGGGLVQPGGSLRLSCAASGSVFSTNTMAWFRQAPGKQREWVGGITPGGFTNYINSVKGRFTISRDNANNTIYLQMSSLRPEDTAVYYCNTLSRMALWGQGTQVTVSS |
| 15 | CA13599 | MAQVQLVESGGGLVQPGGSLRLSCAASGSIFSIYAMGWYRQAPGKQRELVAAITSGGDTNYADSVKGRFTISRDNAKNTVYLQMNSLKPEDTAVYYCNADHTPAGTFQTFDYWGQGTQVTVSS |
| 16 | CA13602 | MAQVQLVESGGGLVQPGGSLRLSCAASGSGFSIYAMGWARQAPGRQRELVAAITSGGSTNYADSVKGRFTISRDNAKNTVYLQMNSLKPEDTAVYYCNADITVVAGSTYDYWGQGTQVTVSS |
| 17 | CA13598 | MAQVQLVESGGGLVQPGGSLRLSCAASGSIFSINAMGWYRQAPGKQRELVAAITSGGRTNYADSVRGRFTISRDHAKNTVYLQMNSLKPEDTAVYYCNAGDWQTMYDYWGQGTQVTVSS |
| 18 | CA13617 | MAQVQLVESGGGLVQAGGSLRLSCAASGIIFHWYDMDWYRQAPGKQRELVATITTGGRADYADFVKGRFSISRDNAKTTVYLQMNSLKPEDTAVYYCSFQDHVLRRWGQGTQVTVSS |
| 19 | CA16072 | MAQVQLVESGGGLVQPGGSLRLSCAASGFPFSTYYMSWVRQAPGKGLEWVSAISPGGDTAYADSVKDRFTISRDSAKNTVYLQMSSLKPEDTAVYYCAAVRFLRAPDLTEKRLYEYWGQGTQVTVSS |

TABLE 4
Sequences of CDR1, CDR2, and CDR3 regions as
annotated in Table 3 for SEQ ID NO: 1-19.

| Full | | | | | | |
|---|---|---|---|---|---|---|
| length | | | | | | |
| ISVD | | SEQ | | SEQ | | SEQ |
| SEQ ID | | ID | | ID | | ID |
| NO: | CDR1 | NO: | CDR2 | NO: | CDR3 | NO: |
| 1 | SGRIFSG | 23 | ITNGGYT | 42 | NALDREA | 61 |
| | NAM | | NY | | RQVDY | |
| 2 | SGSIFSP | 24 | ITSGGYT | 43 | NTLTISW | 62 |
| | TAM | | NY | | S | |
| 3 | SGRTFSR | 25 | ITTSGAV | 44 | AAGQGQL | 63 |
| | YTM | | TTY | | SSQSRFV | |
| | | | | | DY | |
| 4 | SGTIYAI | 26 | ITLGGGT | 45 | KVESSGY | 64 |
| | NRMG | | NY | | TDVF | |
| 5 | SGSNIGY | 27 | IASGGSA | 46 | HADAWYN | 65 |
| | M | | NY | | SRWSDY | |
| 6 | SGDVNYA | 28 | INWRGDN | 47 | AAKSAPY | 66 |
| | V | | TDY | | WYSDIAP | |
| | | | | | D | |
| 7 | SGSIRSI | 29 | ISSGTST | 48 | HVWISGT | 67 |
| | TTS | | NY | | WY | |
| 8 | SGSIRSI | 30 | ISSGTTT | 49 | HAWISGT | 68 |
| | ATS | | NY | | WI | |
| 9 | SGRTFSS | 31 | IRWSGGS | 50 | AASDRGG | 69 |
| | YTM | | TY | | FIATSGG | |
| | | | | | WMDY | |
| 10 | SGGTFST | 32 | VKWSGDM | 51 | AARRGLS | 70 |
| | YAM | | TY | | YYRGPSE | |
| | | | | | YDY | |
| 11 | SGSIFRR | 33 | ITSGGYT | 52 | NLLSEML | 71 |
| | NAM | | NY | | QTV | |
| 12 | SVRTFSF | 34 | IRWTAGS | 53 | AAQRGYL | 72 |
| | YSMG | | TSY | | VEAASRE | |
| | | | | | YDY | |
| 13 | SGLTFGT | 35 | ISGRGGY | 54 | AARQGSP | 73 |
| | YAM | | RDY | | VRSKEEY | |
| | | | | | NN | |
| 14 | SGSVFST | 36 | ITPGGFT | 55 | NTLSRMA | 74 |
| | NTM | | NY | | | |
| 15 | SGSIFSI | 37 | ITSGGDT | 56 | NADHTPA | 75 |
| | YAM | | NY | | GTFQTFD | |
| | | | | | Y | |
| 16 | SGSGFSI | 38 | ITSGGST | 57 | NADITWA | 76 |
| | YAM | | NY | | GSTYDY | |
| 17 | SGSIFSI | 39 | ITSGGRT | 58 | NAGDWQT | 77 |
| | NAM | | NY | | MYDY | |
| 18 | SGIIFHW | 40 | ITTGGRA | 59 | SFQDHVL | 78 |
| | YDM | | DY | | RR | |
| 19 | SGFPFST | 41 | ISPGGDT | 60 | AAVRFLR | 79 |
| | YYMS | | AY | | APDLTEK | |
| | | | | | RLYEY | |

### ASPECTS OF THE DISCLOSURE

A non-natural allosteric modulator of Leucine-rich Repeat Kinase 2 (LRRK2) specifically binding human LRRK2.

Said LRRK2 allosteric modulator, which does not comprise cobalamin derivatives.

Said LRRK2 allosteric modulator, inhibiting or increasing LRRK2 activity.

Said LRRK2 allosteric modulator, specifically binding to a binding site of human LRRK2 that is different from the ATP-catalytic site of LRRK2.

Said LRRK2 allosteric modulator, specifically binding to a binding site of human LRRK2 not exclusively comprising the kinase domain or a binding site different from the kinase domain.

Said LRRK2 allosteric modulator, comprising a small molecule, a chemical compound, a protein, a peptide, a peptidomimetic, an antibody, an antibody mimetic, a single domain antibody, an immunoglobulin single variable domain (ISVD) or an active antibody fragment.

Said LRRK2 allosteric modulator, comprising an ISVD, wherein said ISVD comprises 4 framework regions (FR) and 3 complementarity-determining regions (CDR) according to the following formula (1): FR1-CDR1-FR2-CDR2-FR3-CDR3-FR4 (1).

Said LRRK2 allosteric modulator, comprising an ISVD wherein:
CDR1 consists of a sequence selected from the group of CDR1 sequences of SEQ ID NO: 1 to 19,
CDR2 consists of a sequence selected from the group of CDR2 sequences of SEQ ID NO:1 to 19, and
CDR3 consists of a sequence selected from the group of CDR3 sequences of SEQ ID NO:1 to 19.

Said LRRK2 allosteric modulator comprising an ISVD, said ISVD comprising any of the sequences of SEQ ID NO: 1 to 19, or a sequence with at least 85% amino acid identity thereof, or a humanized variant thereof.

A multi-specific LRRK2 allosteric modulator, comprising at least one of said modulators specified herein.

An in vitro method for detecting the quantity of LRRK2 protein in a sample, the method comprising:
interacting a sample with said LRRK2-specific ISVD, or with said multi-specific agent comprising a LRRK2-specific ISVD; and
detecting the presence or absence or level of said interacted LRRK2-specific ISVD.

A pharmaceutical composition comprising the LRRK2 allosteric modulator, the multi-specific LRRK2 allosteric modulator, a nucleic acid molecule encoding the LRRK2 allosteric modulator or the multi-specific LRRK2 allosteric modulator, or a vector comprising said nucleic acid molecule.

Said pharmaceutical composition, further comprising a compound that is an ATP-competitive LRRK2 kinase inhibitor.

Said LRRK2 allosteric modulator, multi-specific LRRK2 allosteric modulator, nucleic acid molecule encoding the LRRK2 allosteric modulator or multi-specific LRRK2 allosteric modulator, vector comprising said nucleic acid molecule, or the pharmaceutical composition, for use as a medicament, or for use as a diagnostic or for in vivo imaging.

Said LRRK2 allosteric modulator, multi-specific LRRK2 allosteric modulator, nucleic acid molecule encoding the LRRK2 allosteric modulator or multi-specific LRRK2 allosteric modulator, vector comprising said nucleic acid molecule, or the pharmaceutical composition, for use in treatment of a LRRK2-related disorder, specifically for use in treatment of Parkinson's disease.

An in vitro method for detection of the localization and distribution of human LRRK2 protein in a biological sample, comprising the steps of:
reacting a biological sample with said LRRK2-specific ISVD, or multi-specific agent comprising a LRRK2-specific ISVD, and
detecting the localization and distribution of said LRRK2-specific ISVD in said biological sample.

A nucleic acid molecule encoding said LRRK2 allosteric modulator, or said multi-specific LRRK2 allosteric modulator.

A vector comprising said nucleic acid molecule, preferably a viral vector, lentiviral or adenoviral vector.

### REFERENCES

1. Gloeckner, C. J., Boldt, K., Von Zweydorf, F., Helm, S., Wiesent, L., Sarioglu, H. and Ueffing, M. (2010) Phosphopeptide analysis reveals two discrete clusters of phosphorylation in the N-terminus and the Roc domain of the Parkinson-disease associated protein kinase LRRK2. J. Proteome Res. 9, 1738-45.
2. Guaitoli, G., Raimondi, F., Gilsbach, B. K., Gómez-Llorente, Y., Deyaert, E., Renzi, F., Li, X., Schaffner, A., Jagtap, P. K. A., Boldt, K., et al. (2016) Structural model of the dimeric Parkinson's protein LRRK2 reveals a compact architecture involving distant interdomain contacts. Proc. Natl. Acad. Sci. 113, E4357-E4366.
3. Gloeckner, C. J., Boldt, K., Schumacher, A., Roepman, R. and Ueffing, M. (2007) A novel tandem affinity purification strategy for the efficient isolation and characterisation of native protein complexes. Proteomics. 7, 4228-34.
4. Gul, N., Linares, D. M., Ho, F. Y. and Poolman, B. (2014) Evolved Escherichia coli strains for amplified, functional expression of membrane proteins. J. Mol. Biol.
5. Ho, F. Y. and Poolman, B. (2015) Engineering Escherichia coli for functional expression of membrane proteins. In Methods in Enzymology.
6. Rudi, K., Ho, F. Y., Gilsbach, B. K., Pots, H., Wittinghofer, A., Kortholt, A. and Klare, J. P. (2015) Conformational heterogeneity of the Roc domains in C. tepidum Roc-COR and implications for human LRRK2 Parkinson mutations. Biosci. Rep. 35, e00254-e00254.
7. Pardon, E., Laeremans, T., Triest, S., Rasmussen, S. G. F., Wohlkonig, A., Ruf, A., Muyldermans, S., Hol, W. G. J., Kobilka, B. K. and Steyaert, J. (2014) A general protocol for the generation of Nanobodies for structural biology. Nat. Protoc. 9, 674-93.
8. Gloeckner, C. J., Boldt, K. and Ueffing, M. (2009) Strep/FLAG tandem affinity purification (SF-TAP) to study protein interactions. Curr. Protoc. Protein Sci. chapter 19, unit 19.20.
9. Carrion, M. D. P., Marsicano, S., Daniele, F., Marte, A., Pischedda, F., Cairano, E. Di, Piovesana, E., Von Zweydorf, F., Kremmer, E., Gloeckner, C. J., et al. (2017) The LRRK2 G2385R variant is a partial loss-of-function mutation that affects synaptic vesicle trafficking through altered protein interactions. Sci. Rep. 7, 5377.
10. Kandur, W. V., Kao, A., Vellucci, D., Huang, L. and Rychnovsky, S. D. (2015) Design of CID-cleavable protein cross-linkers: Identical mass modifications for simpler sequence analysis. Org. Biomol.

Chem.
11. Liu, F., Rijkers, D. T. S., Post, H. and Heck, A. J. R. (2015) Proteome-wide profiling of protein assemblies by cross-linking mass spectrometry. Nat. Methods.
12. Combe, C. W., Fischer, L. and Rappsilber, J. (2015) xiNET: Cross-link network maps with residue resolution. Mol. Cell. Proteomics.
13. Wauters, L., Versees, W. and Kortholt, A. (2019) Roco Proteins: GTPases with a Baroque Structure and Mechanism. Int J Mol Sci. 20, 147.
14. Marin, I., van Egmond, W. N. and van Haastert, P. J. M. (2008) The Roco protein family: a functional perspective. FASEB J. 22, 3103-3110.
15. Marin, I. (2006) The Parkinson disease gene LRRK2: Evolutionary and structural insights. Mol. Biol. Evol. 23, 2423-2433.
16. Guo, L., Gandhi, P. N., Wang, W., Petersen, R. B., Wilson-Delfosse, A. L. and Chen, S. G. (2007) The Parkinson's disease-associated protein, leucine-rich repeat kinase 2 (LRRK2), is an authentic GTPase that stimulates kinase activity. Exp. Cell Res. 313, 3658-3670.
17. Deyaert, E., Wauters, L., Guaitoli, G., Konijnenberg, A., Leemans, M., Terheyden, S., Petrovic, A., Gallardo, R., Nederveen-Schippers, L. M., Athanasopoulos, P. S., et al. (2017) A homologue of the Parkinson's disease-associated protein LRRK2 undergoes a monomer-dimertransition during GTP turnover. Nat. Commun. 8.
18. Deyaert, E., Leemans, M., Singh, R. K., Gallardo, R., Steyaert, J., Kortholt, A., Lauer, J. and Versees, W. (2019) Structure and nucleotide-induced conformational dynamics of the Chlorobium tepidum Roco protein. Biochem. J. 476, 51-66.
19. James, N. G., Digman, M. A., Gratton, E., Barylko, B., Ding, X., Albanesi, J. P., Goldberg, M. S. and Jameson, D. M. (2012) Number and brightness analysis of LRRK2 oligomerization in live cells. Biophys. J. 102, L41-3.
20. Berger, Z., Smith, K. A. and Lavoie, M. J. (2010) Membrane localization of LRRK2 is associated with increased formation of the highly active Irrk2 dimer and changes in its phosphorylation. Biochemistry 49, 5511-5523.
21. Wu, C.-X., Liao, J., Park, Y., Reed, X., Engel, V. A., Hoang, N. C., Takagi, Y., Johnson, S. M., Wang, M., Federici, M., et al. (2019) Parkinson's disease-associated mutations in the GTPase domain of LRRK2 impair its nucleotide-dependent conformational dynamics. J. Biol. Chem. 294, 5907-5913.
22. Huang, X., Wu, C., Park, Y., Long, X., Hoang, Q. Q. and Liao, J. (2019) The Parkinson's disease-associated mutation N1437H impairs conformational dynamics in the G domain of LRRK2. FASEB J. 33, 4814-4823.
23. Rothbauer, U., Zolghadr, K., Tillib, S., Nowak, D., Schermelleh, L., Gahl, A., Backmann, N., Conrath, K., Muyldermans, S., Cardoso, M. C., et al. (2006) Targeting and tracing antigens in live cells with fluorescent nanobodies. Nat. Methods.
24. Irannejad, R., Tomshine, J. C., Tomshine, J. R., Chevalier, M., Mahoney, J. P., Steyaert, J., Rasmussen, S. G. F., Sunahara, R. K., EI-Samad, H., Huang, B., et al. (2013) Conformational biosensors reveal GPCR signalling from endosomes. Nature 495, 534-8.
25. Steger, M., Tonelli, F., Ito, G., Davies, P., Trost, M., Vetter, M., Wachter, S., Lorentzen, E., Duddy, G., Wilson, S., et al. (2016) Phosphoproteomics reveals that Parkinson's disease kinase LRRK2 regulates a subset of Rab GTPases. Elife 5.
26. Steger, M., Diez, F., Dhekne, H. S., Lis, P., Nirujogi, R. S., Karayel, O., Tonelli, F., Martinez, T. N., Lorentzen, E., Pfeffer, S. R., et al. (2017) Systematic proteomic analysis of LRRK2-mediated rab GTPase phosphorylation establishes a connection to ciliogenesis. Elife 6.
27. Liu, Z., Bryant, N., Kumaran, R., Beilina, A., Abeliovich, A., Cookson, M. R. and West, A. B. (2017) LRRK2 phosphorylates membrane-bound Rabs and is activated by GTP-bound Rab7L1 to promote recruitment to the trans-Golgi network. Hum. Mol. Genet.
28. Sheng, Z., Zhang, S., Bustos, D., Kleinheinz, T., Le Pichon, C. E., Dominguez, S. L., Solanoy, H. O., Drummond, J., Zhang, X., Ding, X., et al. (2012) Ser1292 autophosphorylation is an indicator of LRRK2 kinase activity and contributes to the cellular effects of PD mutations. Sci. Transl. Med.
29. Kluss, J. H., Conti, M. M., Kaganovich, A., Beilina, A., Melrose, H. L., Cookson, M. R. and Mamais, A. (2018) Detection of endogenous S1292 LRRK2 autophosphorylation in mouse tissue as a readout for kinase activity. npj Park. Dis.
30. Wang, S., Liu, Z., Ye, T., Mabrouk, O. S., Maltbie, T., Aasly, J. and West, A. B. (2017) Elevated LRRK2 autophosphorylation in brain-derived and peripheral exosomes in LRRK2 mutation carriers. Acta Neuropathol. Commun.
31. Purlyte, E., Dhekne, H. S., Sarhan, A. R., Gomez, R., Lis, P., Wightman, M., Martinez, T. N., Tonelli, F., Pfeffer, S. R. and Alessi, D. R. (2017) Rab29 activation of the Parkinson's disease-associated LRRK2 kinase. EMBO J. e201798099.
32. Pleiner, T., Bates, M., Trakhanov, S., Lee, C. T., Schliep, J. E., Chug, H., Bohning, M., Stark, H., Urlaub, H. and Görlich, D. (2015) Nanobodies: Site-specific labeling for super-resolution imaging, rapid epitope-mapping and native protein complex isolation. Elife.
33. Muyldermans, S. (2013) Nanobodies: Natural Single-Domain Antibodies. Annu. Rev. Biochem. 82, 775-797.
34. Scott, J. D., DeMong, D. E., Greshock, T. J., Basu, K., Dai, X., Harris, J., Hruza, A., Li, S. W., Lin, S. I., Liu, H., et al. (2017) Discovery of a 3-(4-Pyrimidinyl) Indazole (MLi-2), an Orally Available and Selective Leucine-Rich Repeat Kinase 2 (LRRK2) Inhibitor that Reduces Brain Kinase Activity. J. Med. Chem. 60, 2983-2992.
35. Jaleel, M., Nichols, R. J., Deak, M., Campbell, D. G., Gillardon, F., Knebel, A. and Alessi, D. R. (2007) LRRK2 phosphorylates moesin at threonine-558: Characterization of how Parkinson's disease mutants affect kinase activity. Biochem. J.
36. Deniston, C. K., Salogiannis, J., Mathea, S., Snead, D. M., Lahiri, I., Donosa, O., Watanabe, R., Bohning, J., Shiau, A. K., Knapp, S., et al. (2020) Structure of LRRK2 in Parkinson's disease and model for microtubule interaction. Nature. 588 (7837):344-349.
37. Ramirez, M. B., Ordóñez, A. J. L., Pérez, E., Madero-Perez, J., Gonnelli, A., Drouyer, M., Chartier-Harlin, M.-C., Taymans, J.-M., Bubacco, L., Greggio, E., et al. (2017) GTP binding regulates cellular localization of Parkinson's disease-associated LRRK2. Hum. Mol. Genet. 26, 2747-2767.
38. Deng, X., Dzamko, N., Prescott, A., Davies, P., Liu, Q., Yang, Q., Lee, J. D., Patricelli, M. P., Nomanbhoy, T. K., Alessi, D. R., et al. (2011) Characterization of a selective inhibitor of the Parkinson's disease kinase LRRK2. Nat. Chem. Biol. 7, 203-205.
39. Dzamko, N., Deak, M., Hentati, F., Reith, A. D., Prescott, A. R., Alessi, D. R. and Nichols, R. J. (2010) Inhibition of LRRK2 kinase activity leads to dephosphorylation of Ser 910/Ser935, disruption of 14-3-3 binding and altered cytoplasmic localization. Biochem. J.
40. Kett, L. R., Boassa, D., Ho, C. C. Y., Rideout, H. J., Hu, J., Terada, M., Ellisman, M. and Dauer, W. T. (2012) LRRK2 Parkinson disease mutations enhance its microtubule association. Hum. Mol. Genet.
41. Fell, M. J., Mirescu, C., Basu, K., Cheewatrakoolpong, B., DeMong, D. E., Ellis, J. M., Hyde, L. A., Lin, Y., Markgraf, C. G., Mei, H., et al. (2015) MLi-2, a potent, selective, and centrally active compound for exploring the therapeutic potential and safety of LRRK2 kinase inhibition. J. Pharmacol. Exp. Ther.
42. Watanabe, R., Buschauer, R., Bohning, J., Audagnotto, M., Lasker, K., Lu, T. W., Boassa, D., Taylor, S. and Villa, E. (2020) The in situ structure of Parkinson's disease-linked LRRK2. Cell. 182(6):1508-1518.e16.
43. Schaffner, A., Li, X., Gomez-Llorente, Y., Leandrou, E., Memou, A., Clemente, N., Yao, C., Afsari, F., Zhi, L., Pan, N., et al. (2019) Vitamin B 12 modulates Parkinson's disease LRRK2 kinase activity through allosteric regulation and confers neuroprotection. Cell Res.
44. Jahnichen, S., Blanchetot, C., Maussang, D., Gonzalez-Pajuelo, M., Chow, K. Y., Bosch, L., De Vrieze, S., Serruys, B., Ulrichts, H., Vandevelde, W., et al. (2010) CXCR4 nanobodies (VHH-based single variable domains) potently inhibit chemotaxis and HIV-1 replication and mobilize stem cells. Proc. Natl. Acad. Sci. U.S.A.
45. De Tavernier, E., Detalle, L., Morizzo, E., Roobrouck, A., De Taeye, S., Rieger, M., Verhaeghe, T., Correia, A., Van Hegelsom, R., Figueirido, R., et al. (2016) High throughput combinatorial formatting of pcrv nanobodies for efficient potency improvement. J. Biol. Chem.
46. Fuji R N, Flagella M, Baca M, MA SB, Brodbeck J, Chan B K, Fiske B K, Honigberg L, Jubb A M, Katavolos P, Lee D W, Lewin-Koh S C, Lin T, Liu X, Liu S, Lyssikatos J P, O'Mahony J, Reichelt M, Roose-Girma M, Sheng Z et al. (2015) Effect of selective LRRK2 kinase inhibition on nonhuman primate lung. Science translational medicine 7: 1-13.
47. Taymans J M, Greggio E (2016) LRRK2 Kinase Inhibition as a Therapeutic Strategy for Parkinson's Disease, Where Do We Stand? Curr Neuropharmacol 14: 214-25.
48. Saraiva C, Praca C, Ferreira R, Santos T, Ferreira L, Bernardino L (2016) Nanoparticle-mediated brain drug delivery: Overcoming blood-brain barrier to treat neurodegenerative diseases. J Control Release 235: 34-47.
49. Zipkin M (2019) Exosome redux. Nat Biotechnol 37: 1395-1400.
50. Gloeckner et al. (2006). The Parkinson disease causing LRRK2 mutation 12020T is associated with increased kinase activity. Hum Mol Genet. 15(2):223-32.
51. Leemans, M., Galicia, C., Deyaert, E., Daems, E., Krause, L., Paesmans, J., Pardon, E., Steyaert, J., Kortholt, A., Sobott, F., et al. (2020) Allosteric modulation of the GTPase activity of a bacterial LRRK2 homolog by conformation-specific Nanobodies. Biochem J. 477, 1203-1218.
52. Gardet, A., Benita, Y., Li, C., Sands, B. E., Ballester, I., Stevens, C., Korzenik, J. R., Rioux, J. D., Daly, M. J., Xavier, R. J., et al. (2010) LRRK2 Is Involved in the IFN-γ Response and Host Response to Pathogens. J. Immunol. 185, 5577-85.
53. Kubala, M. H., Kovtun, O., Alexandrov, K. and Collins, B. M. (2010) Structural and thermodynamic analysis of the GFP:GFP-nanobody complex. Protein Sci.
54. Lee, H., Flynn, R., Sharma, I., Carling, P. J., Nicholls, F. J., Stegmann, M., Vowles, J., Wade-Martins, R., James, W. S. and Cowley, S. A. (2020) LRRK2 is recruited to phagosomes and co-recruits Rab8 and Rab10 in human pluripotent stem cell-derived macrophages. Stem Cell reports. 14, 5: 940-955.
55. Eguchi, T., Kuwahara, T., Sakurai, M., Komori, T., Fujimoto, T. and Ito, G. (2018) LRRK2 and its substrate Rab GTPases are sequentially targeted onto stressed lysosomes and maintain their homeostasis 115.
56. Liu, Z., Xu, E., Zhao, H. T., Cole, T. and West, A. B. (2020) LRRK2 and Rab10 coordinate macropinocytosis to mediate immunological responses in phagocytes. EMBO J.
57. Massa, S., Vikani, N., Betti, C., Ballet, S., Vanderhaegen, S., Steyaert, J., Descamps, B., Vanhove, C., Bunschoten, A., van Leeuwen, F. W. B., et al. (2016) Sortase A-mediated site-specific labeling of camelid single-domain antibody-fragments: a versatile strategy for multiple molecular imaging modalities. Contrast Media Mol. Imaging 11, 328-339.
58. Lees, A. J., Hardy, J. and Revesz, T. (2009) Parkinson's disease. Lancet 373, 2055-2066.
59. Elbaz, A., Carcaillon, L., Kab, S. and Moisan, F. (2016) Epidemiology of Parkinson's disease. Rev Neurol 172, 14-26.
60. Tysnes, O. B. and Storstein, A. (2017) Epidemiology of Parkinson's disease. J Neural Transm 124, 901-905.
61. Fahn, S. (2003) Description of Parkinson's disease as a clinical syndrome. Ann. N. Y. Acad. Sci. 991, 1-14.
62. Zimprich, A., Biskup, S., Leitner, P., Lichtner, P., Farrer, M., Lincoln, S., Kachergus, J., Hulihan, M., Uitti, R. J., Caine, D. B., et al. (2004) Mutations in LRRK2 cause autosomal-dominant parkinsonism with pleomorphic pathology. Neuron 44, 601-607.
63. Paisan-Ruiz, C., Jain, S., Evans, E. W., Gilks, W. P., Simón, J., Van Der Brug, M., De Munain, A. L., Aparicio, S., Gil, A. M., Khan, N., et al. (2004) Cloning of the gene containing mutations that cause PARK8-linked Parkinson's disease. Neuron 44, 595-600.
64. Monfrini, E. and Di Fonzo, A. (2017) Leucine-rich repeat kinase (LRRK2) genetics and parkinson's disease. In Advances in Neurobiology, pp 3-30.
65. Di Maio, R., Hoffman, E. K., Rocha, E. M., Keeney, M. T., Sanders, L. H., De Miranda, B. R., Zharikov, A., Van Laar, A., Stepan, A. F., Lanz, T. A., et al. (2018) LRRK2 activation in idiopathic Parkinson's disease. Sci. Transl. Med. 10, eaar5429.
66. Gilks, W. P., Abou-Sleiman, P. M., Gandhi, S., Jain, S., Singleton, A., Lees, A. J., Shaw, K., Bhatia, K. P., Bonifati, V., Quinn, N. P., et al. (2005) A common LRRK2 mutation in idiopathic Parkinson's disease. Lancet 365, 415-416.
67. Barrett, J. C., Hansoul, S., Nicolae, D. L., Cho, J. H., Duerr, R. H., Rioux, J. D., Brant, S. R., Silverberg, M. S., Taylor, K. D., Barmada, M. M., et al. (2008) Genome-wide association defines more than 30 distinct susceptibility loci for Crohn's disease. Nat. Genet. 40, 955-62.
68. Hui, K. Y., Fernandez-Hernandez, H., Hu, J., Schaffner, A., Pankratz, N., Hsu, N. Y., Chuang, L. S., Carmi, S., Villaverde, N., Li, X., et al. (2018) Functional variants in the LRRK2 gene confer shared effects on risk for Crohn's disease and Parkinson's disease. Sci. Transl. Med. 10, eaai7795.
69. Marin, I., van Egmond, W. N. and van Haastert, P. J. M. (2008) The Roco protein family: a functional perspective. FASEB J. 22, 3103-3110.
70. Marin, I. (2006) The Parkinson disease gene LRRK2: Evolutionary and structural insights. Mol. Biol. Evol. 23, 2423-2433.
71. Civiero, L., Vancraenenbroeck, R., Belluzzi, E., Beilina, A., Lobbestael, E., Reyniers, L., Gao, F., Micetic, I., de Maeyer, M., Bubacco, L., et al. (2012) Biochemical Characterization of Highly Purified Leucine-Rich Repeat Kinases 1 and 2 Demonstrates Formation of Homodimers. PLoS One 7.
72. Sen, S., Webber, P. J. and West, A. B. (2009) Dependence of leucine-rich repeat kinase 2 (LRRK2) kinase activity on dimerization. J. Biol. Chem. 284, 36346-36356.
73. Liao, J., Wu, C.-X., Burlak, C., Zhang, S., Sahm, H., Wang, M., Zhang, Z.-Y., Vogel, K. W., Federici, M., Riddle, S. M., et al. (2014) Parkinson disease-associated mutation R1441H in LRRK2 prolongs the “active state” of its GTPase domain. Proc. Natl. Acad. Sci. U.S.A. 111, 4055-60.
74. Li, X., Tan, Y. C., Poulose, S., Olanow, C. W., Huang, X. Y. and Yue, Z. (2007) Leucine-rich repeat kinase 2 (LRRK2)/PARK8 possesses GTPase activity that is altered in familial Parkinson's disease R1441C/G mutants. J. Neurochem. 103, 238-247.
75. Lewis, P. A., Greggio, E., Beilina, A., Jain, S., Baker, A. and Cookson, M. R. (2007) The R1441C mutation of LRRK2 disrupts GTP hydrolysis. Biochem. Biophys. Res. Commun. 357, 668-671.
76. Luz6n-Toro, B., de la Torre, E. R., Delgado, A., Perez-Tur, J. and Hilfiker, S. (2007) Mechanistic insight into the dominant mode of the Parkinson's disease-associated G2019S LRRK2 mutation. Hum. Mol. Genet. 16, 2031-2039.
77. West, A. B., Moore, D. J., Biskup, S., Bugayenko, A., Smith, W. W., Ross, C. A., Dawson, V. L. and Dawson, T. M. (2005) Parkinson's disease-associated mutations in leucine-rich repeat kinase 2 augment kinase activity. PNAS 102, 16842-16847.
78. Berwick, D. C., Heaton, G. R., Azeggagh, S. and Harvey, K. (2019) LRRK2 Biology from structure to dysfunction: Research progresses, but the themes remain the same. Mol. Neurodegener., Molecular Neurodegeneration 14, 1-22.
79. Scott, J. D., DeMong, D. E., Greshock, T. J., Basu, K., Dai, X., Harris, J., Hruza, A., Li, S. W., Lin, S. I., Liu, H., et al. (2017) Discovery of a 3-(4-Pyrimidinyl) Indazole (MLi-2), an Orally Available and Selective Leucine-Rich Repeat Kinase 2 (LRRK2) Inhibitor that Reduces Brain Kinase Activity. J. Med. Chem. 60, 2983-2992.
80. Domingos, S., Duarte, T., Saraiva, L., Guedes, R. C. and Moreira, R. (2019) Targeting leucine-rich repeat kinase 2 (LRRK2) for the treatment of Parkinson's disease. Future Med. Chem. 11, 1953-1977.
81. Baptista, M. A. S., Dave, K. D., Frasier, M. A., Sherer, T. B., Greeley, M., Beck, M. J., Varsho, J. S., Parker, G. A., Moore, C., Churchill, M. J., et al. (2013) Loss of leucine-rich repeat kinase 2 (LRRK2) in rats leads to progressive abnormal phenotypes in peripheral organs. PLoS One 8, e80705.
82. Andersen, M. A., Wegener, K. M., Larsen, S., Badolo, L., Smith, G. P., Jeggo, R., Jensen, P. H., Sotty, F., Christensen, K. V. and Thougaard, A. (2018) PFE-360-induced LRRK2 inhibition induces reversible, non-adverse renal changes in rats. Toxicology 395, 15-22.
83. Dianne Bryce, Christopher M Ware, Janice D Woodhouse, Paul J Ciaccio, J Michael Ellis, Laxminarayan G Hegde, Sabu Kuruvilla, Matthew L Maddess, Carrie G Markgraf, Karin M Otte, Frederique M Poulet, Lauren M Timmins, Matthew Kennedy, M. J. F. (2021) Characterization of the onset, progression, and reversibility of morphological changes in mouse lung following pharmacological inhibition of LRRK2 kinase activity. J Pharmacol Exp Ther. Online ahe.
84. Bogoyevitch, M. A. and Fairlie, D. P. (2007) A new paradigm for protein kinase inhibition: blocking phosphorylation without directly targeting ATP binding. Drug Discov Today 12, 622-33.
85. Lu, X., Smaill, J. B. and Ding, K. (2019) New Promise and Opportunities for Allosteric Kinase Inhibitors. Angew Chem Int Ed Engl. 59 (33): 13764-76.
86. Hudry, E. and Vandenberghe, L. H. (2019) Therapeutic AAV Gene Transfer to the Nervous System: A Clinical Reality. Neuron 101, 839-862.
87. Saraiva, J., Nobre, R. J. and Pereira de Almeida, L. (2016) Gene therapy for the CNS using AAVs: The impact of systemic delivery by AAV9. J. Control. Release 241, 94-109.
88. Hocquemiller, M., Giersch, L., Audrain, M., Parker, S. and Cartier, N. (2016) Adeno-Associated Virus-Based Gene Therapy for CNS Diseases. Hum. Gene Ther. 27, 478-96.

1. An allosteric modulator of Leucine-rich Repeat Kinase 2 (LRRK2) which specifically binds human LRRK2, wherein the allosteric modulator, when bound to LRRK2, prevents the association of the bound LRRK2 with microtubules in cells.

2. The LRRK2 allosteric modulator of claim 1, wherein the K_{D} value of the allosteric modulator for binding LRRK2 is about 200 nM or lower.

3. The LRRK2 allosteric modulator of claim 1, which affects LRRK2 kinase activity in cells and/or in vitro.

4. The LRRK2 allosteric modulator of claim 1, wherein the allosteric modulator comprises a small molecule, a chemical compound, a protein, a peptide, a peptidomimetic, an antibody, an antibody mimetic, a single domain antibody, an immunoglobulin single variable domain (ISVD), or an active antibody fragment.

5. The LRRK2 allosteric modulator of claim 1, wherein the allosteric modulator comprises an immunoglobulin single variable domain ISVD), wherein the ISVD comprises 4 framework regions (FR) and 3 complementarity-determining regions (CDR) according to the following formula (1): FR1-CDR1-FR2-CDR2-FR3-CDR3-FR4 (1).

6. The LRRK2 allosteric modulator of claim 5, wherein the CDR1, CDR2 and CDR3 regions are selected from those CDR1, CDR2 and CDR3 regions of a sequence selected from the group of sequences of SEQ ID NO: 1 to 19, wherein the CDR regions are annotated according to Kabat, MacCallum, IMGT, AbM, or Chothia.

7. The LRRK2 allosteric modulator of claim 5, wherein:
CDR1 consists of a sequence selected from the group of CDR1 sequences of SEQ ID NO: 23-41,
CDR2 consists of a sequence selected from the group of CDR2 sequences of SEQ ID NO: 42-60, and
CDR3 consists of a sequence selected from the group of CDR3 sequences of SEQ ID NO: 61-79.

8. The LRRK2 allosteric modulator of claim 6, wherein the ISVD comprises any of the sequences of SEQ ID NO: 1 to 19, or a sequence with at least 85% identity to the full length thereof, or a humanized variant thereof.

9. The LRRK2 allosteric modulator of claim 1, wherein the allosteric modulator inhibits LRRK2 kinase activity in cells.

10. The LRRK2 allosteric modulator of claim 1, wherein the allosteric modulator inhibits LRRK2 substrate phosphorylation in cells.

11. The LRRK2 allosteric modulator of claim 1, wherein the allosteric modulator increases LRRK2 kinase activity in cells.

12. The LRRK2 allosteric modulator of claim 1, wherein the allosteric modulator prevents LRRK2 association to microtubules in a cell.

13. The LRRK2 allosteric modulator of claim 1, wherein the allosteric modulator is comprised in a multi-specific or multivalent binding agent.

14. A nucleic acid molecule encoding the LRRK2 allosteric modulator of claim 1.

15. The nucleic acid molecule of claim 14, wherein the nucleic acid molecule is comprised in a vector.

16. An in vitro method for detecting LRRK2 protein in a sample, the method comprising:
i) interacting a sample with the LRRK2 allosteric modulator of claim 5, and
ii) detecting the presence or absence or protein level of said LRRK2-specific ISVD binder bound to LRRK2; or
wherein the sample is a biological sample,
i) reacting a biological sample with the LRRK2 allosteric modulator of claim 5, and/or a labelled form of the allosteric modulator, and
ii) detecting the localization and distribution of the LRRK2 allosteric modulator of claim 5 in said biological sample.

17. (canceled)

18. The LRRK2 allosteric modulator of claim 1, wherein the allosteric modulator is comprised in a pharmaceutical composition.

19. The LRRK2 allosteric modulator of claim 18, wherein the pharmaceutical composition further comprises an ATP-competitive LRRK2 kinase inhibitor compound.

20. (canceled)

21. A method of treating of a LRRK2-related disorder in a subject, the method comprising administering to the subject the LRRK2 allosteric modulator of claim 1 or a nucleic acid encoding the LRRK2 allosteric modulator.

22. The method of claim 21, wherein the LRRK2-related disorder is Parkinson's disease or Crohn's disease.

23. (canceled)


SEQUENCE LISTING (79 sequences)

SEQ ID NO: 1 - protein, 120 aa, Artificial Sequence
  Nanobody CA12610
MAQVQLVESGGGLVQAGGSLRLSCVASGRIFSGNAMGWYRQAPGRQRELVAGITNGGYTNYRQFVKGRFTISRDNAKRTVYLQMNSLKPEDTAVYYCNALDREARQVDYWGQGTQVTVSS

SEQ ID NO: 2 - protein, 116 aa, Artificial Sequence
  Nanobody CA12618
MAQVQLVESGGGLVQAGGSLRLSCAASGSIFSPTAMGWYRQAPGKERELVAGITSGGYTNYRNFARGRFTISRDNAKKTVYLQMNSLNPEDTAVYYCNTLTISWSWGQGTQVTVSS

SEQ ID NO: 3 - protein, 125 aa, Artificial Sequence
  Nanobody CA13612
MAQVQLVESGGGLVQAGGSLRLSCAASGRTFSRYTMGWFRQAPGKDREFVARITTSGAVTTYADSAKGRFTISRDNAKNTVYLQMNTLKPEDTAVYYCAAGQGQLSSQSRFVDYWGQGTQVTVSS

SEQ ID NO: 4 - protein, 119 aa, Artificial Sequence
  Nanobody CA14135
MAQVQLVESGGGLVQPGGSLRLSCVASGTIYAINRMGWYRQAPGKQRELVALITLGGGTNYGDSVKGRFTISRDNAKNTGYLQMNSLKPEDTAVYYCKVESSGYTDVFWGQGTQVTVSS

SEQ ID NO: 5 - protein, 119 aa, Artificial Sequence
  Nanobody CA13606
MAQVQLVESGGGLVQPGGSLRLSCAASGSNIGYMAWYRQAPGSQRELVARIASGGSANYADSVKGRFTISRDNVKNTVYLHMNSLKPVDTAVYYCHADAWYNSRWSDYWGQGTQVTVSS

SEQ ID NO: 6 - protein, 123 aa, Artificial Sequence
  Nanobody CA14259
MAQVQLVESGGGLVQAGGSLRLSCAASGDVNYAVGWFRQAPRKEREFVAAINWRGDNTDYTDSVKGRFTISRDNAKNTVYLQMNSLKPEDTAVYYCAAKSAPYWYSDIAPDYWGQGTQVTVSS

SEQ ID NO: 7 - protein, 117 aa, Artificial Sequence
  Nanobody CA14130
MAQVQLVESGGGLVQPGGSLRLSCAASGSIRSITTSAWFRQPPGKQRELVAGISSGTSTNYADSVKGRFTISRDNAKNTVYLQMNSLKPEDTAVYYCHVWISGTWYSGQGTQVTVSS

SEQ ID NO: 8 - protein, 117 aa, Artificial Sequence
  Nanobody CA14133
MAQVQLVESGGGLVQPGGSLRLSCAASGSIRSIATSAWYRQPPGKQRELVAGISSGTTTNYADSVKGRFTISRDNAKNTVYLQMNSLKPEDTAVYYCHAWISGTWISGQGTQVTVSS

SEQ ID NO: 9 - protein, 127 aa, Artificial Sequence
  Nanobody CA13611
MAQVQLVESGGGLVQAGGSLRLSCAASGRTFSSYTMGWFRQAPGKEREFVAAIRWSGGSTYYADSVKGRFTISRDNAKNTVYLQMNSLKPEDTAVYYCAASDRGGFIATSGGWMDYWGKGTQVTVSS

SEQ ID NO: 10 - protein, 126 aa, Artificial Sequence
  Nanobody CA14134
MAQVQLVESGGGLVQAGGSLRLSCRASGGTFSTYAMGWFRQVPGKERETVAAVKWSGDMTYYADSVKGRFTISRDNAKNTVYLQMNSLRPEDTAVYYCAARRGLSYYRGPSEYDYWGQGTQVTVSS

SEQ ID NO: 11 - protein, 118 aa, Artificial Sequence
  Nanobody CA12614
MAQVQLVESGGGLVQPGGSLRLSCAASGSIFRRNAMGWYRQVTGKERELVAGITSGGYTNYRDFVKGRFTISRDNAKNTVYLQMNNLKPEDTAVYYCNLLSEMLQTVWGQGTQVTVSS

SEQ ID NO: 12 - protein, 126 aa, Artificial Sequence
  Nanobody CA14136
MAQVQLVESGGGLVQAGGSLRLSCEASVRTFSFYSMGWFRQAPGKEREFVAAIRWTAGSTSYADIVKGRFTISRDNGKNTVVLQMNSLEPEDTGVYYCAAQRGYLVEAASREYDYWGQGTQVTVSS

SEQ ID NO: 13 - protein, 129 aa, Artificial Sequence
  Nanobody CA13620
MAQVQLVESGGGLVQAGGSLRLSCATSGLTFGTYAMAWFRQAAEKEREFVAFISGRGGYRDYADSVKGRLTIFRDNAKNNAKNTVYLQMNNLKPEDTAVYYCAARQGSPVRSKEEYNNWGQGTQVTVSS

SEQ ID NO: 14 - protein, 116 aa, Artificial Sequence
  Nanobody CA14131
MAQVQLVESGGGLVQPGGSLRLSCAASGSVFSTNTMAWFRQAPGKQREWVGGITPGGFTNYINSVKGRFTISRDNANNTIYLQMSSLRPEDTAVYYCNTLSRMALWGQGTQVTVSS

SEQ ID NO: 15 - protein, 123 aa, Artificial Sequence
  Nanobody CA13599
MAQVQLVESGGGLVQPGGSLRLSCAASGSIFSIYAMGWYRQAPGKQRELVAAITSGGDTNYADSVKGRFTISRDNAKNTVYLQMNSLKPEDTAVYYCNADHTPAGTFQTFDYWGQGTQVTVSS

SEQ ID NO: 16 - protein, 122 aa, Artificial Sequence
  Nanobody CA13602
MAQVQLVESGGGLVQPGGSLRLSCAASGSGFSIYAMGWARQAPGRQRELVAAITSGGSTNYADSVKGRFTISRDNAKNTVYLQMNSLKPEDTAVYYCNADITVVAGSTYDYWGQGTQVTVSS

SEQ ID NO: 17 - protein, 119 aa, Artificial Sequence
  Nanobody CA13598
MAQVQLVESGGGLVQPGGSLRLSCAASGSIFSINAMGWYRQAPGKQRELVAAITSGGRTNYADSVRGRFTISRDHAKNTVYLQMNSLKPEDTAVYYCNAGDWQTMYDYWGQGTQVTVSS

SEQ ID NO: 18 - protein, 117 aa, Artificial Sequence
  Nanobody CA13617
MAQVQLVESGGGLVQAGGSLRLSCAASGIIFHWYDMDWYRQAPGKQRELVATITTGGRADYADFVKGRFSISRDNAKTTVYLQMNSLKPEDTAVYYCSFQDHVLRRWGQGTQVTVSS

SEQ ID NO: 19 - protein, 127 aa, Artificial Sequence
  Nanobody CA16072
MAQVQLVESGGGLVQPGGSLRLSCAASGFPFSTYYMSWVRQAPGKGLEWVSAISPGGDTAYADSVKDRFTISRDSAKNTVYLQMSSLKPEDTAVYYCAAVRFLRAPDLTEKRLYEYWGQGTQVTVSS

SEQ ID NO: 20 - protein, 10 aa, Artificial Sequence
  6xHistidine and EPEA C-terminal tag
HHHHHHEPEA

SEQ ID NO: 21 - protein, 2527 aa, Homo sapiens [sequence omitted]

SEQ ID NO: 22 - protein, 55 aa, Artificial Sequence
  N-terminal SF tag (flag and twin-strep)
MDYKDDDDKGSAASWSHPQFEKGGGSGGGSGGGSWSHPQFEKGARSTSLYKKAGF

SEQ ID NO: 23 - protein, 10 aa, artificial sequence
  CDR1 of Nb CA12610
SGRIFSGNAM

SEQ ID NO: 24 - protein, 10 aa, artificial sequence
  CDR1 of Nb CA12618
SGSIFSPTAM

SEQ ID NO: 25 - protein, 10 aa, artificial sequence
  CDR1 of Nb CA13612
SGRTFSRYTM

SEQ ID NO: 26 - protein, 11 aa, artificial sequence
  CDR1 of Nb CA14135
SGTIYAINRMG

SEQ ID NO: 27 - protein, 8 aa, artificial sequence
  CDR1 of Nb CA13606
SGSNIGYM

SEQ ID NO: 28 - protein, 8 aa, artificial sequence
  CDR1 of Nb CA14259
SGDVNYAV

SEQ ID NO: 29 - protein, 10 aa, artificial sequence
  CDR1 of Nb CA14130
SGSIRSITTS

SEQ ID NO: 30 - protein, 10 aa, artificial sequence
  CDR1 of CA14133
SGSIRSIATS

SEQ ID NO: 31 - protein, 10 aa, artificial sequence
  CDR1 of CA13611
SGRTFSSYTM

SEQ ID NO: 32 - protein, 10 aa, artificial sequence
  CDR1 of CA14134
SGGTFSTYAM

SEQ ID NO: 33 - protein, 10 aa, artificial sequence
  CDR1 of CA12614
SGSIFRRNAM

SEQ ID NO: 34 - protein, 11 aa, artificial sequence
  CDR1 of CA14136
SVRTFSFYSMG

SEQ ID NO: 35 - protein, 10 aa, artificial sequence
  CDR1 of CA13620
SGLTFGTYAM

SEQ ID NO: 36 - protein, 10 aa, artificial sequence
  CDR1 of CA14131
SGSVFSTNTM

SEQ ID NO: 37 - protein, 10 aa, artificial sequence
  CDR1 of CA13599
SGSIFSIYAM

SEQ ID NO: 38 - protein, 10 aa, artificial sequence
  CDR1 of CA13602
SGSGFSIYAM

SEQ ID NO: 39 - protein, 10 aa, artificial sequence
  CDR1 of CA13598
SGSIFSINAM

SEQ ID NO: 40 - protein, 10 aa, artificial sequence
  CDR1 of CA13617
SGIIFHWYDM

SEQ ID NO: 41 - protein, 11 aa, artificial sequence
  CDR1 of CA16072
SGFPFSTYYMS

SEQ ID NO: 42 - protein, 9 aa, artificial sequence
  CDR2 of Nb CA12610
ITNGGYTNY

SEQ ID NO: 43 - protein, 9 aa, artificial sequence
  CDR2 of Nb CA12618
ITSGGYTNY

SEQ ID NO: 44 - protein, 10 aa, artificial sequence
  CDR2 of Nb CA13612
ITTSGAVTTY

SEQ ID NO: 45 - protein, 9 aa, artificial sequence
  CDR2 of Nb CA14135
ITLGGGTNY

SEQ ID NO: 46 - protein, 9 aa, artificial sequence
  CDR2 of Nb CA13606
IASGGSANY

SEQ ID NO: 47 - protein, 10 aa, artificial sequence
  CDR2 of Nb CA14259
INWRGDNTDY

SEQ ID NO: 48 - protein, 9 aa, artificial sequence
  CDR2 of Nb CA14130
ISSGTSTNY

SEQ ID NO: 49 - protein, 9 aa, artificial sequence
  CDR2 of CA14133
ISSGTTTNY

SEQ ID NO: 50 - protein, 9 aa, artificial sequence
  CDR2 of CA13611
IRWSGGSTY

SEQ ID NO: 51 - protein, 9 aa, artificial sequence
  CDR2 of CA14134
VKWSGDMTY

SEQ ID NO: 52 - protein, 9 aa, artificial sequence
  CDR2 of CA12614
ITSGGYTNY

SEQ ID NO: 53 - protein, 10 aa, artificial sequence
  CDR2 of CA14136
IRWTAGSTSY

SEQ ID NO: 54 - protein, 10 aa, artificial sequence
  CDR2 of CA13620
ISGRGGYRDY

SEQ ID NO: 55 - protein, 9 aa, artificial sequence
  CDR2 of CA14131
ITPGGFTNY

SEQ ID NO: 56 - protein, 9 aa, artificial sequence
  CDR2 of CA13599
ITSGGDTNY

SEQ ID NO: 57 - protein, 9 aa, artificial sequence
  CDR2 of CA13602
ITSGGSTNY

SEQ ID NO: 58 - protein, 9 aa, artificial sequence
  CDR2 of CA13598
ITSGGRTNY

SEQ ID NO: 59 - protein, 9 aa, artificial sequence
  CDR2 of CA13617
ITTGGRADY

SEQ ID NO: 60 - protein, 9 aa, artificial sequence
  CDR2 of CA16072
ISPGGDTAY

SEQ ID NO: 61 - protein, 12 aa, artificial sequence
  CDR3 of Nb CA12610
NALDREARQVDY

SEQ ID NO: 62 - protein, 8 aa, artificial sequence
  CDR3 of Nb CA12618
NTLTISWS

SEQ ID NO: 63 - protein, 16 aa, artificial sequence
  CDR3 of Nb CA13612
AAGQGQLSSQSRFVDY

SEQ ID NO: 64 - protein, 11 aa, artificial sequence
  CDR3 of Nb CA14135
KVESSGYTDVF

SEQ ID NO: 65 - protein, 13 aa, artificial sequence
  CDR3 of Nb CA13606
HADAWYNSRWSDY

SEQ ID NO: 66 - protein, 15 aa, artificial sequence
  CDR3 of Nb CA14259
AAKSAPYWYSDIAPD

SEQ ID NO: 67 - protein, 9 aa, artificial sequence
  CDR3 of Nb CA14130
HVWISGTWY

SEQ ID NO: 68 - protein, 9 aa, artificial sequence
  CDR3 of CA14133
HAWISGTWI

SEQ ID NO: 69 - protein, 18 aa, artificial sequence
  CDR3 of CA13611
AASDRGGFIATSGGWMDY

SEQ ID NO: 70 - protein, 17 aa, artificial sequence
  CDR3 of CA14134
AARRGLSYYRGPSEYDY

SEQ ID NO: 71 - protein, 10 aa, artificial sequence
  CDR3 of CA12614
NLLSEMLQTV

SEQ ID NO: 72 - protein, 17 aa, artificial sequence
  CDR3 of CA14136
AAQRGYLVEAASREYDY

SEQ ID NO: 73 - protein, 16 aa, artificial sequence
  CDR3 of CA13620
AARQGSPVRSKEEYNN

SEQ ID NO: 74 - protein, 7 aa, artificial sequence
  CDR3 of CA14131
NTLSRMA

SEQ ID NO: 75 - protein, 15 aa, artificial sequence
  CDR3 of CA13599
NADHTPAGTFQTFDY

SEQ ID NO: 76 - protein, 14 aa, artificial sequence
  CDR3 of CA13602
NADITVVAGSTYDY

SEQ ID NO: 77 - protein, 11 aa, artificial sequence
  CDR3 of CA13598
NAGDWQTMYDY

SEQ ID NO: 78 - protein, 9 aa, artificial sequence
  CDR3 of CA13617
SFQDHVLRR

SEQ ID NO: 79 - protein, 19 aa, artificial sequence
  CDR3 of CA16072
AAVRFLRAPDLTEKRLYEY